SEQUENCE LISTING (155 sequences)

SEQ ID NO: 1 - DNA, 38000 bp, Homo sapiens
gagtcaatgtccagcctggagtgcaatggtgcggtatggcttactgcagcctcaaacccctaaactcagatgatcctcccacctcagcctcccaaatagctgggactacaggtacatgccaccatgccagctaactttttttacattttattttttgtagagatgggggtcttgcaattattgcccaggctggtctcaaactcctggcctcaagtgatcctcccaccttggcctccaaaagcattgggattacaggcatgagccactgtgcttggctcaaagctgctttaaaaatttatgtacatatatatattttaagacagagacttgctctactgcactggctgtagtgcagtggcacaatcatggctcactgcagtctcaaacttctgggctaaagcaatcctcccgcttcagcctcccaagtagctgggactacagttgcatgccaccacccccagctaatttttaaattttttgtagagacagggtcttgctatgttgtccagactggtctcaaactcctgggctcaagcaatctgcctgcttcagcatccgcaagtgttggggttacagatgtaagccactgcgcccacgagttgctgctgaatatccaaattgtctaagcttctcctctgggtttaaaatggtctatggcatgtctctacctataacctcttgccccaggcatcttttctgagcaatgtcctgattttaggtaagagatacagcatcttgcatcagttcttccaggatcccccagacaagaacagatgcacgtaatagtttgcaaataaggcctgctctctttggaggagggagctgagaactgtactactgttgtctcaattccaaaactgttgactgagtgcagtggctcacgcctgtaatcccaacactttgggaggccaaggcaggaggatcacttgaggccaggagtttgagaccagcccagacaacatagtgagaccctatctctacaaacaatttaaaacactagctgggtgtggtggcacatacatgtaattctagcttctcaggagacggaggttggaggattgcttgagcccaggagtttgaggctgcagtaagccatgattgtaccaatacattccagcctgggctacagaatgagaccctgcttcaaaaagaaaaaaaaaaaaaaaagaccaagactgctgccatgctggggaaggggtggggcaagactaagtaaaaacaccacaaaactttgctactgttttgaagatggccttttttaaattgagtgtttgcctggttgctgtaggcctttgttttctagagtgacaacaaagttggttctgacagtttggcttgtttattcagtgtttcagtttggaaatgagagcttggagcttcctaggccaccattttgctgatgtcatttccaatggcattttttgcatctcgactttttcctcgcgttcaatgcttcaggaccacaagatggttgctacagctctagaccttccatctgtctagtgtggcgaaaagtggggaaggctagaatatcatgccagctgcatacctcccctttcatgagggaagaaaaagccttcccacggggatcacagggcccctgctagctgcaaaggggtctgggagaacagggagagcctctctcacctgagcagtggacacaatccttcaccaaagagtgcaggttctgatggcaagaaagacaaaggggccaccggcaggctcgttaccccaaagagcgagaagtaggggatgtgattacttacatctgtaccagttagagtgttgtacacatatccagccaaggtacctgtggcccaggtcaggtgactggcttagcaatttcacctaccttcctctcagcccagatccccaaattctttgaatgctgttgggatgcagaacagcaagtcagcgagtgattttttttaatttaatttttatgagtacacagtagattatatatttatggggtacatcagatattttgatacagatatacaatgtgtcataatcacatcaggttgtaaatggagtgaccgtcacctcaagcatttgtcacttctctgttacaaacattttaattacacccttttagttattttaaaatgtactgctgattgtaattaccctgttatgctatcaaataccagctcttattcattctatctaattatatttttgtacccaccaaccatctccgcttccccctacctccccactactcttcccagcctctggtaaccatcgttctacctactgtctattgccatgcgtttgttttcatttttagctcctataaatgagtgaaaacacatgaagtttgtctttctgtgcctggcttatttcagtgagtgatcctcatgtctccagggcttgtctgtacatgactcacctggggcagcctctgccaggtgtcaccccggagccagcaacaaagggctgctctgctgatggctgcctcacccccggctgctccctcagtgaactggcacagctctgggcccctctcgggaccttctcagagtagccacatttcagacctgtcttatgattctaacatcaaacttataatatcaatcttactaataccaatagaaagtggaaaatgaggtattatctggcagtcattaaattagtaagttctaatgacaaacataatacacgatgaaggtgagactgtgggaagatggtgcctttgcgagttgcccatgtcagtggtaagagtcacggccctcgggaaatcaccgagtcttcattacccaagactggcatcaacccttcaccaaattccaataactgagaatctgataattacccaataaatcctagattagcctgaggaaagaaatgagctgtccacgtaagagtcgtaaacattgggccggggctggtggctcatgcctgtaatcccagcactttgggaggccgaggcaggcggatcacgagatcaggagatcgagaccatcctggctaacatggtcaaaccctgtctctactaaaaatacaaaaatgaaccaggcatggtggcacatgcctgtagtcccagctactcgggaggctgaggcaggagaatcatttgaacccaagaggcagaagttgcagtgagccgagattgcgccactgcactccagcctggcaacagagtgagactctgtctcaaaaaaaaaaaaaaaaaaaaaagaatggtaaacattgtactctgactcacaaatctcatctaggggaacttgttttaaggaaataaattcaaagaaggaggaaacattgttaggtgcaaagaagtcaaccagaaacttatttatcaaaaatgaactattgggaaccggctcgacagtcagcaccagaagaggagaagatccacgcgttctgtggaccataacctagtcacggacgtgctgatcagagattgaaggcaacagggaggatttatgtgaaaagtcaagagaaaaagcaggatgcatgtacatatcatatggttacagctcggcacgtgtgtccagaggcaccggcagctgggttgggagatcgggtgtgaaattttcactgtcattccgagccggattgtgccgctgttatgctgcgtgtgtttcacaaatgaccccaggagaccacatagctggactctatctctctgtggtgctagactgggcacagctgggctccaggggcttagcctagacagcccccatgggaagaaacatatgaaaggcagggtgggcctttcatatctttgttctgacacagctctgtgcatgccgacagtgtcttcttgtcgcaagtgcccacggccctgcctaaggccctttgacactgaaggtgcccgccacgtgctggggcgaaatcttccaggaatgtcctctaccagtgacagatgaatgtggtggaaagctgtctgtgtccttattccttggaggggaccttcttgggcacgtccccaccagttcccggaggtccctgggggcaggagcaagctcttggatgcattctggtcagctttcttccatcccctggctcattccccattcaccgactgctgtcatctggggtcatctccccaataaactctttgcactgggatccttgtttcaggatctgtttctggaggaactagatgacaacaccgggaacagaggacctagagaggcagcttcatgggtggtggggtgtccgcctctgccggccagggacttgggagcagtgctgggaaggtgctggatggagctgtcactcacaggggcaggtccttggctgctgactgtcttcctctccactatggctgtcttgagaacttaggggtcagcctgaccctgccttggcccccttcctctcagcctctgtcttctcctgcatgaggctgggtggctcccctgtgaatcaggcaggggtccacagaacactagagacaggtcccttcctgcagctgtctccagtaggtggccacgcaggagatgttcccaacaagctgcccttatctgcagctcagctttggtaatgggggcccattaccaaatgggggtaaaggtcatggcccatcctggtgatagtgagaacccaaggtaggccttgaagattcctatcaggagggagcagaaagtgtgtaccacacccctgggcccaggtggagcagggctgctgctcaaggctcccagccatgctctgtcccttgctaggggtgaccggtgggacaggcctgggcaagggacaagagggagaaggtcggggggaagaggggatgaagagcaaagtgagcaaaggagagtcttccactatctggggtctctgtcaactgtcaggccctagagtgagctgttctttccctttgcttcctggaggaggggacttttgtcactgcgtcactccaccctgcctgcccctccgttatcaggctgttaatattaattaacaacagttgctagggatgacagtgcagagggttcctctgagcccattgctggccctggtcccaagagggggtagggcagagctggggtctgaggctgagccagggagggtgcggaggttcctcggccatgctgagctcctgaggccgggtcccagccagtgcctggtcccatctgtgcctccaggccctggcaccaactccagcagtgttaggggctaatagcgtggtctctcccctagctgactcagccctctggcttcggtcgctttgggaagtgagtggagaccctagcacctgcgtgatgaggctcatctaaagcgggggcctgtggactggggccaaacagtgggagtggtggatcattaaccagcagggctcagcctcattggtccctaacccagtcaggccagggttgtcatcgaaggggaggaggctgccttaatgtgtgttcagcccttggctgttcctgaggcctggcctggctccccgctgaccccttcccagacctgggatggcggaggccggcctgaggggctggctgctgtgggccctgctcctgcgcttggtgagtcccagggcttggctccacctcccctgcggcctccagttagggaccctggggccagccgtgtaccaggcgagcgttactgggtgacagcaagggagcctcagggcctgcgggctgggcaagtctctggacacatgagggatgccaggccccacagaggaggggtgcaggtggagggtttccaggttacaggcttgaatgcacacaggggtgaaagaggctgctggactggggtgctccaagtccctcctgtcactggccctactgtggggtccaggcctgcagttgagggaggtctgaggcaaggaggtgctgggatggggttacctggtgagcatcacctagggaggactgagcactctggaggctgggagaagatccagcgctggcacctcttaagttcctcgcttactttgtgtctgggaggtgggtgacagcttttggcctcaagcaggtggtggtagtggtggtgggagtcggggggcctcctgaacagactctccatgagagaccctggcctctggatgtggtgtacagtgtggggactcaggctgactttgacgtgggcagagcccgggaccttggagtcagctttgcctccttacccatctctggcctctccagcatgactttcctaagctgcaggtctatcaggccacccccaggaagaaaggccagtgttgtcactccaacactggctggctggcacatgcctccaggaggcttcctactccccacactccccgcttccctgcccctgctccatgtccttcttaccctcacaccctccctggctgcctgctgcctggatggcacccagctgtgtcagggcccacgcgtgatgttgctgtgctctgcaggcccagagtgagccttacacaaccatccaccagcctggctactgcgccttctatgacgaatgtgggaagaacccagagctgtctggaagcctcatgacactctccaacgtgtcctgcctgtccaacacgccggcccgcaagatcacaggtgatcacctgatcctattacagaagatctgcccccgcctctacaccggccccaacacccaagcctgctgctccgccaagcagctggtatcactggaagcgagtctgtcgatcaccaaggccctcctcacccgctgcccagcctgctctgacaattttgtgaacctgcactgccacaacacgtgcagccccaatcagagcctcttcatcaatgtgacccgcgtggcccagctaggggctggacaactcccagctgtggtggcctatgaggccttctaccagcatagctttgccgagcagagctatgactcctgcagccgtgtgcgcgtccctgcagctgccacgctggctgtgggcaccatgtgtggcgtgtatggctctgccctttgcaatgcccagcgctggctcaacttccagggagacacaggcaatggtctggccccactggacatcaccttccacctcttggagcctggccaggccgtggggagtgggattcagcctctgaatgagggggttgcacgttgcaatgagtcccaaggtgacgacgtggcgacctgctcctgccaagactgtgctgcatcctgtcctgccatagcccgcccccaggccctcgactccaccttctacctgggccagatgccgggcagtctggtcctcatcatcatcctctgctctgtcttcgctgtggtcaccatcctgcttgtgggattccgtgtggcccccgccagggacaaaagcaagatggtggaccccaagaagggcaccagcctctctgacaagctcagcttctccacccacaccctccttggccagttcttccagggctggggcacgtgggtggcttcgtggcctctgaccatcttggtgctatctgtcatcccggtggtggccttggcagcgggcctggtctttacagaactcactacggaccccgtggagctgtggtcggcccccaacagccaagcccggagtgagaaagctttccatgaccagcatttcggccccttcttccgaaccaaccaggtgatcctgacggctcctaaccggtccagctacaggtatgactctctgctgctggggcccaagaacttcagcggaatcctggacctggacttgctgctggagctgctagagctgcaggagaggctgcggcacctccaggtatggtcgcccgaagcacagcgcaacatctccctgcaggacatctgctacgcccccctcaatccggacaataccagtctctacgactgctgcatcaacagcctcctgcagtatttccagaacaaccgcacgctcctgctgctcacagccaaccagacactgatggggcagacctcccaagtcgactggaaggaccattttctgtactgtgccaagtgagtccatggtggggcccaagcgaggagtgggctggggctggggctgggctgccatggcctcctgggaacctggccgggcatacagctggtcctgaaggaccagaggtagctattcctacggctctggcctggggccgcccagatgattatctctgcccctcgtccggccgccatttcctttggtcagagttcctgctcatggctgcaggtttgtgcgtggccatcgctggcccttcaaccccgagtccactctgtctttctgcagatttcttgacatgtgggagctccctgccacactcttgctttaagtctgacagaggagcccgattggcagagtacatatttatatttgctatgttttgcttcttgtttctgtgccaggggccgtagggccatcagtaacccatgaggtaccatggtatgcattggaaaaggtgccctcaggccagaggtcgtggctggtctcaggcacctgggccgggtgtcctggggtaggccacagccacacacacttctattgattggggttcggtctttggttctgtccactctggtgtgctgccaacaagatgccaacaacgctgctgggccaagggggccaagagccaagggcagcagcagggccttggcagtggaggctccttgaggttggagtagagcagaggtcctcaagatgaacgtttagtactccatactccagagcaaatgagagttaaaaggggcaaatagcatcttagtgttattatgaaaacagttctgaccttacagaccctggaaagggtctccaggacgcctaagggccccaggccacactttgagaaccactggattggaagagagtgccgacactttctgtcccctgctacctggctctgcatccctcagctgggccccaagtttgggctgcttcccagagtgtctgtgccaggaacccaagggctctctcttggaaatagcaggaacgagaggagccattgtttgctctggggaggcatcatggtctgacctcagactcatgtctgacggtagctttatagtccattatagggtattatctttattttgacttcggatgctcacaacaactctcgggtggtccaattatctccattttacagacaggaaaactgaggttcagaggggtgtggtaagctgctcaaggtcacacagcaaccagcactcgcttgctgagatctgagagaggggggtagagagctttgctcaggtgtcccactgcatcttcgcaatgacgggctttgcagaaagggctaagctgaaggacctacagacttgcctgagggcaccagtctagtaaactgtgaaaacattggctgctgggctccagggttccaaatctaacctcaatacctaaagggtttcgggggccctaggcaggagaaggaggctgagagggcaacgtttgagacagcccatgccagaccccatggctcaaatcccagctcttccaccctcacgggacttcaggtgtgacgctcaatccagagtcagataatgtcagagccaggaaggtcaggccagtgtgtggagacatgagaggctcagagggacaggtcccggagcagcccctgcctgccacagagaaggcactcagggcagctccaactcactccgtgggtgggggcctgcaggagatcttgctggatgggagccatttaggacccactcggctgggtcctaaatagctaaatggcctaaatgcagatagctgggctatctgcagccagtgtcccccaccccaccagctcaccctccatagtgctgtgggtctggggtgggaggggaagggaggggccatagggactgggcagggccaggaaaggccctttccctttgcggtcatctccctctagtgccccgctcaccttcaaggatggcacagccctggccctgagctgcatggctgactacggggcccctgtcttccccttccttgccattggggggtacaaaggtaagctaagtgggccctgagaggaagccaaggaagatgcagtattggggcaggaaccatagacgggagggtgggagtggtgctggggattctcgcggcctgggggtagcctggcttctggaagctgtaggccaaccctgtcctgtttcctctctctgccatctcctttatcttctagtagtgttactcaggcactgtggtttttctgcctgggcccaaaggtctcgcctttggctgagagaagtggggtgtaggaggtaaggccatgtatcagatgaggaaggagtgggggagaaggagcaaggggtgatgggaggggtgcagctagatagggggagggaatataggggtgcagctggagggggagggaggcacgggtgcagcaggaagggtctgagtatttcttatcccaggaaaggactattctgaggcagaggccctgatcatgacgttctccctcaacaattaccctgccggggacccccgtctggcccaggccaagctgtgggaggaggccttcttagaggaaatgcgagccttccagcgtcggatggctggcatgttccaggtcacgttcatggctgaggtaggggctgcagggtccctggctctgggggtgcaacccaggtggtcttgggtcagttcctgtgtccccatcctggccctggcccttcctaagtgaccctgggcagtggctgcctgctcagaacggggtgattgtgatggctgttcttatagcctcacctgcgattatagggggccatcaggccctatgacacaacacacaattagtgcccagtgaccgagctattgagagctggcctggctgaagcaggcacggtcagtgggggctggtcgggtgtgtgtccacagcgctctctggaagacgagatcaatcgcaccacagctgaagacctgcccatctttgccaccagctacattgtcatattcctgtacatctctctggccctgggcagctattccagctggagccgagtgatggtgagaagcgggagggacacagctaagtgggctagcccaggaccccaggcatcttcagtaggccttctacaactttcctaaccacagcacctcagaacagcaaagtggacacacccaagtggctgccccaaagggtaatacctcttgcaagtgttctgtgctgaaaggtcaagagcaattttcttttcttttcctttctttttcttctcttttctttgcttttcttttctcttctcttttccctcctaccctctctttctctttcttttctttctctctctgtttctctttttctctctttctttcttttgagacagggtcttgctctgttgcccaggctggagtgcagtggcatgatcttagctcactgcaacctcaaaactcctgggcacaagtgatcctcctgcttcagcctcccaagtagttgggactataggcacttgccattgtgcccagctattttttttttttttttgagacagagctttgctcttgttgcccaggctgcagtgtaacggcgcgatctcggctcactgcaacctccgcctcctgggttcaacaattgtcctgcctcagcctcccgagtagctggcattacaggcatgtgtcaccacgcctggctaattttgtgtttttagtagagatggggtttctccatgttggtcagactggtcttgaactcctggcctcaggtgatccgcccacccaaagtgctgggattacatgcgtgagctaccacgtccggccattttttttgttttgtagtttttgtagagatggggtctcgctttttgcctaggctggtctcaaactcctgggctcaagtgattcttcctcatcagcctcccaaaatgttgagattacaggtgtgagccagcacacctggcctaagagcagttttctgtctgttacatgccataccctcacttgcccaaatgcaaagctaagacttaaaatctcttgcaatgcatgctcaaggaagatggagtaggctcacccatgcctttgggtttcctggacctccccttgggaggatggctctgcagaggggctttaatgtgagatgtgagctcctcaccactgggggcagtatcgggcacctgcaggcactgagggtgcctgccggctactttgtctggcctagctgaggctggtgggcatactgggtaggtgctaagtggctagggggctgagcctgtttgcattgcaggtggactccaaggccacgctgggcctcggcggggtggccgtggtcctgggagcagtcatggctgccatgggcttcttctcctacttgggtatccgctcctccctggtcatcctgcaagtggttcctttcctggtgctgtccgtgggggctgataacatcttcatctttgttctcgagtaccaggtaagaagggaggagctctccacacccccaactgcccactcttctcccaacctcacctcctggcctgatgggactctggcgtgaatttgctgggtctccctgcagactctttctgttcatcgacacgcatgtttacaatatctgtagaaactagagtgtgttgacataaatgacttcatcctgcctctaccatctggaattagctttctgttaaccccttgcaatgtctagtaaaacctctccatgttagtacattacagcctcctcctgtctttatgctgctaggtagcattccatggtaaggataaatcagagtcgatttcacctctccctgttggtgaacaattagggttccaacagtgcttggaacagggatgctatagacatctcaaatgcaccaaccatttctcccagccagaccctggaagaagaatattggccatggagagtatgagagtctctgatgattcaggaaggtcagagcagctcctcaggcctggctgcagctctgggcacttgccaactccctgctggcctttgaggggcggtgcccttggagggccctggctcttatccctgctgttcccacacagaggctgccccggaggcctggggagccacgagaggtccacattgggcgagccctaggcagggtggctcccagcatgctgttgtgcagcctctctgaggccatctgcttcttcctaggtgagcctgggtgagacctccccactcggcattaggcttgctgggttagtgccggggcctaggagttcccagagggcagtgggtatagtgcagattcccttccccctgcaccctgtcaatgtcggctaccactctgcccttgaagccagggtgccctgacagccctctgctccctcacaggggccctgacccccatgccagctgtgcggacctttgccctgacctctggccttgcagtgatccttgacttcctcctgcagatgtcagcctttgtggccctgctctccctggacagcaagaggcaggaggtaggggcagctgggccagtactgagggacctgcccctgggttcccaccatggcagggagatggggtggctttaccaccacagagatggcccagagaatggggtgggggacaggggcattgtgccaggagagtaatatttaggccatgtattctccaatttcctacagaaaaataaatttgttttgacaattttttaaatataatcaaacctcctaaagtgcatgatgttgagaaataaaatacagttgacccttgaacaatgtggagattagggcaccgactgtctaagcagttgaaaatctgcatgtaactttttttttttttgagacggagtttcactctgtcacccaggctggagtgcaatggcgtgatatcagctcaccacaacctctgcctcccgggttcaagcgattctcctgcctcagcctcccaattactgggattacaggccccctcctcctgcacgcctggctaatttttgtgtttttaatagagatggggtttcaccatgttggtcaggttggtctcgaactcctgacctcaggtgatctgcccaccttggcctcccaaagtgctggcgtgagccaccatgcctggtctgcatgtaacatttgacccttctaaacttaattcctactagcctactattgactggaagccttaatgataacataaatagtcgataacacatcttttgaatgttatatgtattataaactgtattcttacaataaaggaagcaagaaaaaagaaaatgttagtaagaaaatcataaggaagagaaaatctatttactattcacgaagtgaaagtggatcatcatgagggtcttcatcctcgtcgtcttcaggttgagtaggctgaggaagaggaggaagaggagggcttgatcttgctgtttcaggggcggcagaggtggaagagaatccagggataagtgagcccaggcagttcaaactcgtgttgttcaagggtcagctgtataaatgagaggtcgacaggagttgatctgttggttcccatgatggtgtaaaatttaaagatattttatcaagattaaaataaaagcaaagaaaacagcacactggtatgtctccatgagggcactggcacgggccacccacagaaggtgacactccctgggggcaagaaggtggtccctggggccttgtctgctctgggactaccttgagggggtgcctcccactccaggcctcccggttggacgtctgctgctgtgtcaagccccaggagctgcccccgcctggccagggagaggggctcctgcttggcttcttccaaaaggcttatgcccccttcctgctgcactggatcactcgaggtgttgtggtgagtgggcctcgaaccacacgagagcaggggcactaggtggggacctcgcctcagggagagcagggttggaggtggggaggttgcctaggcccaaatgctgatacttggggctggcacgcaagtctgctcaactccagaatgttgcccatgacaccctgactgacttaaatttgtggggagatgggggacggctgttgggcagggtggtctcatgcagcaggtcccttctcagctgctgctgtttctcgccctgttcggagtgagcctctactccatgtgccacatcagcgtgggactggaccaggagctggccctgcccaaggtgagcccaggcccttctcaacccttaggcccctgggatttggggaggggcagtagcaaccagcagggatgggttggggggtcctccggccaggggcttggccagaggtgcagaattgttcattactctggaggcacctccagcagtcctggggagtgaagccacattcgtgtatgaacagcacaacagccaggtgccagccccaggccacagtaagagagatggcccaggcatcggagggctgtccatgtgagatggcaggccacaaagaatgactgccactttgctgagtgcctgcccagtgtccagccctgcgaattctctgggcctgaagcccggggagggcaggggttcaggggaaggaaagccccgtggttggaggggacctccaaggtcacataggatttgcagaggaaagtgatgacagactcgccagtgggaggctagggtgagcccaggtgtgtttcctgggcgtggcagcgactgtgggggtgggatgagctggaggccaagggcatggtcggggagagtgctgattgcccagcctggaccagtaagtgtgcgggccaacaggcacaatgcatcagccaaggctggggacccggctcctctggatatgcatcagcggtggccatgggctggtggccaagaggaagcagccacagacaacaaagtctgagacacatggtcagactgcatgagcaagctctagggagagggaaggcatcgaggggactcgatgtctaggtcccatctggggaactgtgatggaggtttgggcaagggtctgggtactggcaggagccccagtggaagcagccaggcctgagcccacaacagggctgagtggggtgcggctggggtaggtgtgttaggcagtactggcctggggtcctggaagccaggtgagggaggacaagagcagatggctcaggactgtactttgggtgactttatggagggagagcaggtgaggagtcacagaatgaacctgccacctgcagaagccctgggggctatgtcacagggctgaggtgaagagggtctctagtgccccaagagcaagaaggaaggatgtgatgggctgccagaccctgctgaggttttatgttgatgtcttttgtttatttttctgttggggacatttgtttcttactgcttttaaaaattttatcattttttttccgttttttattgtggtaaaatacacataatagaaaattaccattataaccatttttaagtgtacagttcagtgatattaagtacactcatactattcaactatcaccaccatccatctccaaaactctttcctttttgcaaaattgaaactttacccaacaaacagtgactccccattctcccctcccctcagcccctgacacaaccaccttttatttatttatttattttgaaacagagtttcactcttgttgcccaggctggagtgcaatggtgtgatctcggctcaccgcaatctccgcctcccgggttcaagtgattctcctgcctcagcctcccaagtaactgggattacaggtggccgccaccacgcccagctaatttttgtatttttagtagagacagggtttcaccatgttggcctggctggtcttgaacttctgacctcaggtgatccaccagccctggcctcccaaagtgctgggattacaggtgtgagccaccgcacctggcctctactttttctttttttttttgagatggagtcttgctctgtcacccaggctggagtgcaatggtgcagactcggcttactgcagcctccacctcccaggttcaagcgattctcctgactcaacctcctgagtagctgggactacagccgtgtgccaccactcccagctaatttttgtacttttagtagagacagggtttcgccatgttggccaggctggtctcgaactctggaccttgtgatctgcctgctttgccctcccaaagtgctgggattacaggcatgaaccactgtgcccggcccatttactttctgttctatgagtttgaccactctaggcacctcaggtaagtgaactcatacaatatttatttttttggctgggagtggtggctcactcctgtaatcccagcactttgggaggctgaggcaggcagatcacctgaggtcaggagtttgagaccagcttgaccaacatggagaaaccccatctctactaaaaatacaaagttaactgggcatggtggcacatgcctgtaatcccagctactcaggaggctgaggcaggagaatcacttgaacctgggaggcagaggttgtggtaaactgagatcacgccattgcactccagcctgggtaacagagtgaggattcgtctcaaaaaaaaaaaaaaagtatattttgtctgatcttagtatagctacccctattctcttttggttactatttacatggaatatcttttttctgttcttccactttcaatctatttgtgtttttggacctaaggtgagtctcttggagacagcatatagttagatcacgttttgctgttttttagcagatgggggctgcctagggcacagtatgctgactctcacaatctcgatcgtgtgtgtgtgtgtgtgtgtgtgtgtgtgtgtgtgtgtgtgtgtgtgtgtgtgtttaattcattctaccactcttttttttcttttttttttttgagatggagcctcagtctgtcacccaggctggagtgcagtggagcgatctcagctcactgcaacttacacctcccgggttcaagcaattctcctgcctcagcctcctgagtagctgggattataggtgcatgccaccatgcctggctaatttttttgtattttttgtagagacagggtttcaccatgttggccaggctggcctcaaactcctgactttgagtaatccacccacctcggcttcccaaagtgctgggattacaggcgtgagccaccatgcctggtcctactactctcttttgattggagagtttaatccatttacatttacagtaattattgataaggagggatttacttctgtcattttgctatttgttttctatatgccttgtagattttttgtttctcatttcctgcattactgacttattttgtgcttagttgattgctactagtgaaattttacattttccttctcattttcttttgtgcatagtctacagctaattttatttgtgattaccatggggattatcttaaatgtgctgaagttataacactctaaatttatgccaactttgtttccatagcatacaaaaactctgccctataacaactccatcttacctccctttcagttattgatgtcacaaaattatatcttgagctagccatggtggcttatgcctgtaatcccaatgctttgagaggtggaggcaagaggattgcttgaggccaggaatttgaggccagcctagccaacacagtgagatcccatctctagaaaaaatttaaaatttagctgggcaagatggcacgtgcctgtagtcccagctatgtgggaggcttgcttgagtccaggaattcaagtatgcagtcagctatgatcatgccactgtactccagcctgagcaacagagagacaccttgtctcaaaaaattttatttttcagctgggtgtagtggctcatggctgcaatcccagcactttgtgaggtggttggatcacttgaggccaggaggtcaagattagcctggccaacatgacaaaaccccatctctactaaaaatacaaaaattagccaggcatggtggcacacaactgtaaacctagctacttgggaggctgagacatgagaattgcttgaatctaggaggtagaggttgcagtgagctgggatcgtaccactgtactccagcttgggcgacagagcgagactatgtctcaaaaacttttgtatttttatgcattatgtatccaaaatcataggctaatgattttttttgcatgagtctcttaaatcatgtacaaaaaggtggagttataaatcataacatttataactgcccatttatttacctttgccagggatttatttatttatttaaagaggcagagtcttgctctgttgcccaggctgagatgcagtggtgtgatcatagctcactataacctcaaactcctggcctcaaaagatcctctcacctcagccacctgaagtactgggattacaggtgtaagccactatgcctagccaagggatttttatttcttcatacatctttgagttactgctgatgtctttttttttttttttttttttttttgagaagttgttttgctcttgttgcccacccaggctggagtgcagtggcatgatctcagctcaccgcaacctctgcctcccgagttcaagcgattctcctgcctcagccccccgagtactgggattacaggcatgtgccaccacgccaggctaattttgtatttttagtagagatggggtttctccatgttggtcaggctggtcttgaactcccaacctcaggtgatctgcccgccttggcctcccaaagtgctgggattacaggcatgagccaccatgcctggcctgtctaatgtctttttatttcaacctacaggattccttttagcatttctttcagggaaggtctagtgataacgaattccttcagcttttgtttatctgagaatgtcttaatttcaccctcattttaattttttaaaattttttatttattttgagatggagtttcactcttgtcgcccaggctggagtacaatggtgtgatctcagctcactgcaacctctgcctcctgggttcaagtgattctcctgcctcagcctcctgagtagctgagattacaggtgcatgccaccatgccaggctaatttttgtatttttaatagagacggggttttaccatgttggccaggctggtcatgaactcctgacctcaggtgatccacccaccttggcctcccaaagtgctaggattacaggtgtgagccactgtgcccggcccatttttattttttaattaaaacaatttttttgagatgggggtctcactgtgttactcaggctggtctcgaacttttgggctcaggtgatcctcgtgtctcagcctcccaaagtattgggattataggacgaatcacctcatctggcatctccctcatttttatttttaatttttagttttttttttttttttttgagatggagtctcactgtcacccagattggagtgcggtggtgtgatctcggctcactgcaacctccacctcccaggttcaagagattctactacctcagcctccaaagtagctgggattacaggtgcatgcctccacgcctggctaatttttgtatttttagcagagatggggtctcaccatgttagtcaagctggtctcaaactcctggcctcaaataatctgtctgcctcggcctcccaaagtgctgggattacaggcatgagccaccatgcctggccttctccctcatttttaagtgacagttttgctggaattaggattcttcattgacaattgttttttcttcagcacttgttttttgttgttgttgtttgtttttgagacagagtctcactctgtcatccaggctggagtgcagtggcatgatctcagctcactgcaacctctgcttctcaggttcaagtgattctcctgcttcatcctcctgagtagctgggattacaggtttgtgccaccatgcctggctaatttttgtattttcagtagagatggggttttgccatgttggccaggctggtctcaaactcctgacctcaggtgatccacctgcctcagcctcctgaagtgctgggattacaggcatgagccatcatgcccagcattcttcagcactttcaatctacaaacccactgccatctgggcttcaaggtttctgatgagaaatatgctgataatctttttgaggatcttttgtatatgccaagtcacttcttttttttcaatattttctattttttaaaaaacttattttattttactttttatttttattttttagaggcagggtcttgctatgttgcctagaatggacttgaaaccctgggctcaagcaatcctcccacctcagcctcttgagtagctgggactacaggtatatgccaccatgcctggcttgtctttggtttttgacagctaaattataatatccagctgggtgcagtggcttatgcctgtaatcccagcactttgggaggccaaggtgggtggatcacaaggtcaagagatcaagaccatcctggccaacatggtgaaaccccatctctactaaaaatacaaaaattagctgggcatggttgtgcgcacttgtagtccaagatacttgggaggctgaggcaggagaatcacttgaacccaggaggcagaggttgcagtgagccgagattgtgccactgcactccagcctggcaacagagcaagactccatctcaaaaaaaaaaaattataatatccgttggtgtgggtttctttagtttatcctattggagtttattgagtttcttgaatgtttatattcatgtctttcatcaaatttggggagttctggccataattttttcaaataatctcacttcccctttctcttttcttctggaattcttacaattcatattttggtctatttgatgatgatgatgtctgacaggtcccttaggctctgctctgttcactttcgttattttttttcctttctcttcttcagactcagtaatttcaatggtcttatcttcagtttgctaattctttcttctgactgcttttgaatccctctagtgaatttttcatttaagttactgtactttttagctccagagtttttttgctcttttttatgtttcctcctcattgatatttccattttgttcataaatttttccttgactttgttttcttttagctctttgagcaactttaaggcaattgttttattcatttattttattatttatttatttattttttgagacagagtcttgctctatcacccaggctggagtgcaatggtgtgatctcgggtcactgcaacctctgcctcctggggttaagcctcagcctcccaagtagctgggattacaggtgcctgccaccatgcttggctaatttttgtatttttagtagagacagggtttcaccatattagccaggctggtctcgaactcctggcctcatgtgatctgcctgccttggccttctgatgttgtgggattacaggcatcagccactgtgcctggctgagacaattgttttgaagtctttgtctagtaagtctgctgtctggtcttacccaggaacagtttctgttggttaatattttccctttgaatgggccatgtttttctttttcttggtgtgtttttggttgaaaaatggacatttgattcttataatgtggtagctctggagatcagattctccttctttcccagggcttgctttattttatttattgctgttggtgtttctgtgctggggatcagccaaaggcacagagttaatgtcttctcaggtatttttgagactgcatttttctctgagcatttatgcagtgtggtgactgtctaaatatccctatatttatggttgcttttgaatgtccttgtccttatatgtatggttcccaaaaggagaaaaagggaaaaatgaaggtgtcggggataggtgcttactctttaaatctcctggaagtcactttagtaagatgtggaggtggttgcaacaacggtggtgggagttgcattagtggctgcctgcctgtgtatctgtaccaccaatatcagaagtaatgatcaattatcagaactcagatccttgatatttgaacttatttatttatttattagagacagggtctggctctcttgctgaggctgaagtgcagtggtgcaatcataggtcactgcagcagcaaacttccaggctcaaatgattctcctatttcagcctcctgagtagctaggactacaggcatgtgccaccacacccagctaacttttgtatttttttttgtagagacagggtgtcgctatgtgcccagatcggtctcccactcttgggctcaagtgaccctcctgcttgccctcccaaagttctgaaattacaagtgtaagccatcatgcccagctgatatttggtggatggtgtccttgcctacctggctcctgcaagctgtgtacaagctgcttctggaaagcatacacagctgcatgccttgaggctgggagtggcaaatgggtagctgctactgtactaaagctgagattgcctgaaattaaccacaatttactgtccaagccttatcctggaagcttccagccctcaatagactccagagttccaaaatcgttacactagggccggtgtggtggctcatgcctgtaatcccagccatttgggaggccgagacgggtggatcacttgaggtcaggagtttgagacaagcctggccaacatggtgaaaccccatctcttttaaaaatacaaaaatcagctgggagcggtggcacatgcctgtaatcctagctactcaggaggctgaggcacaagaatcgcttgaacccaggaggcggaggttgcagtgagcagagatcgcgccactgcactccagcccagaagactccatccatctcaaaacaaaacaaaacaaaacaaaaacaaaatagttataccagacaaattgttgtctagctggggagagggattcctgacacttcctactgtgccattttccctaatgtcactctgagcctttatgttatagaagggagcagaccatgaggatgcctggtgcatggctttgagggtgtgcacactgacatttatatgtgcacacaaatatgggccgttgtcacaggccagcttgttagacggtggctgtgccatattgggggtgataggaaggggtacaattatgtgtctgtgcatgtttgtgtgtgtcagtgtgtgttcatgtgaggtgataggtgttgctctgtgtttgtacctgcataagtgtacttctgtttgcacctgtgattatacctattctgtgaaccttggagtatgttcatctgggggtacacctaaaactgtgttccggtgtaactgtacagtgcacatacatcttgagggtacccctgagtgtgtgtgtctgtgcatgtccttctctatatgtaccttgtgtgtgacctctgagcatgtacatctctgtgtatattttgtgtacttgtgtgcatgtacctctgtgtacctctaagcatgtatctacgtgtatatctctgagtgtcccactgagcacatccctttgagtgtgtaactgcatgtgtgtctctgaacatgttcctctgtgtgttcctctgatcatggacctctgaacatgtgccttttagcatgtacctctgtgtgtaccttcgagagtgtgagctggattgagccctttaggggtgtgcatagcgaaccaaagctcactgaccctcctccactcctaggactcgtacctgcttgactatttcctctttctgaaccgctacttcgaggtgggggccccggtgtactttgttaccaccttgggctacaacttctccagcgaggctgggatgaatgccatctgctccagtgcaggctgcaacaacttctccttcacccagaagatccagtatgccacagagttccctgagcagtgagttcctggcccgccccaaaccccagcctactccctgtttgagtccctccagtcctctccagtcccctcttcctgatgttctatccctgtcctgctgccctgctgccttgctgccgtatgcctggggagggctgcgtgggggttgggccacgagaaggacccaccaccctgcccagctggccttttcacccttcctcccacctgccccttaggtcttacctggccatccctgcctcctcctgggtggatgacttcattgactggctgaccccgtcctcctgctgccgcctttatatatctggccccaataaggacaagttctgcccctcgaccgtcagtgagtgtggggccatggggactcactgtccaccacagctcgggcaaactgaggcaacagaaaggagaggactggagaggctccctcaacctctcccacgcatcctgcagggtctgtcgggggcatgggtgcagatgtggcctgagggacaggcactctgtgagaagcacctgtgtgggtgaccgtgctggcccgtgggcatcacacatgtatactgctgtgtactgtgcccccattttcagagcacatggtgctcccgggtggcagggcagtggggagtcaggaggggagagctgctgaggttagcacatggccctgccgcccaaagcagtggcatttgtaggtggagaggcctttgtggggcctgtttttctgccccaaacttcctttccccttctgcctgtaggtgcccacagtttctatagccaagaggagaacttctcccacaaatgacaaatgcaaatccccctagaagcgactggttgaggctggagtgcccaggacctttgatgggattgttggggaaggaggggcacaaagcaggagctgctggccctggggtgtcactgcccagacccctgctttctctgcagactctctgaactgcctaaagaactgcatgagcatcacgatgggctctgtgaggccctcggtggagcagttccataagtatcttccctggttcctgaacgaccggcccaacatcaaatgtcccaaagggtaagcttgggagggccttctgctggggaggacagacatgtgggacacaggatggggttgaatatagagaggcaggaggaggctatcaggggcctctctggggtggctgtgggctgggcagatgaaagaagcttcgtccctggctaagcctttgccctgaccttcttgcagcggcctggcagcatacagcacctctgtgaacttgacttcagatggccaggttttaggtaagcatggccttgcctggaggggaggacataaatcggttgctctggagggcccccgaaaaccccagggaacagcctgtcacatgttgtctccctcctttgtcaggaggttctcactgcgctggccctgtcagcaggggtcttgtttcccagctccacatctcagacttcaccccttctctcactcccaagtccatggtcagtgctaagtttgtggaattgattcagcagttgataccatacttgggagttctccacaccctggctaagcacctttcttaccagcacaaattacacccaaagggcagctggttaaatgaattaggatgcttggcacagcacaatcctagcagtcatttaaagtaacaagaggctgggcgcctgtaatcttagcactctaggaggccaaggcgagaggatctcttgaatccaggagtttgagaccagcccaggcaacagtagggagaccctcttttttttttttcgagacggagtctcgctttgttgcccaggctggagtgcagtggtgcaatctcggctcactgcaacctccactttccgggttcaagcgattctcctgcctcagcctcctgagtagctgggactataggagcataccatcatgtctgggtaatttttgtattttcagcagagatggaattgcaccacgttggccaagctggtctcaaactcctgacctcaggtgatatgcctgccttggcctcccaaagtgctagtattacaggcatgagccactgtgcccggcctcctctacaaagtaaaatttaaaaaattgcccgggtgtggtggcgtgtgcctgtagttccagctattcagaaggctgggcgggaagaatgcctgagtctgggaggttgaggctgtagtgaactgtgatcgcaacactgcactccagcctgggcaacaaagtgagaccctctctcaaaaaaaaaaagaaagaaaaaagtaacaagagagatgcagttggactgacaggaaaaggacccacaacatgctgtcagcttatacagcagatggcagaacaagacagccatctgtgtaaaggagctggccatagctccgtgcagacatgctcggtgtaggggccctaagggagctcgtgctggagatggacatgggggtcgtcggtgggtgggggagtttttgaaggatgatctcactttgtactgaaataattcatagtttgaactgctggctgaaagctgcctcaagttcgctcaccccacccttccagctatgaagttcccatgtttccagaagggcaatgcaccctgcccagccctggtagctgagcacaacaggctctgtgaggccagtgtggtggggctggtgtggacagatgggagtggatgtgtcagtcagggaatgaggagcagggcctggaaggagcacacagtagagccaagcccccataaccgggggcaagtctgcaccatctctgacctttgtcttcttgtgtgtgcactaggttagtctagagcagcacttcccaaaatgaggtcccccagccagcagcatcagcataacctggaaattgttcaaaatgaagttccagctaggtgctgcagctcacgcctataatcccagtacgttgggaggccaaggtgggaggatcacttgagcccaggagtctagtctgtctgagaccagcctgggcaaaaaagccagatattgaaagaaaagaagagagaagaaaaggaaagaaaagaaaagaaaagaaagaaagggagaaagagagagagagaaagagagagaaagagaaagaaagaaagaaggaaggaaggaaggaagaaaaagaaagaaagaaagaaaaagaagaaacgcaagttctcagccctcacccaagactttgcagaccccgaattgctgggctgggctgggcatttgtgtgtgaactaccctccaggtggtcagaggcctggtgggaagttctccaggcacctcccctgctctgagattgtatgtatccaagaacatttctcttcttttttctccacacctatgtagcactattgtttctttttcagatacacatgctcactgtacacaataaagaaataactttttttttttttttgagacacagttgccattctgtcacccaggctggagtacagtggcacaatctcggctcactgcaacctctacctcctggattcaagtaattctcctgcctcagcctccctagtagctgggattacaggcacatgccactatgctcagctaatttttgtattattaatagaggcagagtgtcgccaagaaacaacctttttgggccaggtgcggtggctcacacctgtaatcccagcagtttgggagaccgaggccggcgaatcacttgaggtcaggagtttgagaccagcctggccaacatggtgaaaccctgtctctactaaaaatacaaaaattagccaggcatggtggcatgcacctgtaatcccagctacttgggaggctgaggcaggacaatcacttgaacccgggaagcagaagttgcagtgagccaagatcgcaccactgcactccagctgcggtgacagtgagactctgtctcgaaaacaaaaacaagaacaaaaaaccctttattgtataaaggtcttaataaccttaatttcttcttttttttttttgagatgggatcttgctctgttgcccagctggagtgcagtagcatgatctcagctcactgcagcctctgcctcctgagttcaagaattctcctgcctcagccccccaagtagctgggattacaggggtgtgccaccacgcctggctaatttttgcatttttagtagagacagggtttcaccatgttgggcaggctggtcttgaactcctgacctcaggtgatcgacctgccttagccttccaaagtgctgggattacaggcatgagccaccacacccggccaataaccttaatttcttaaaagtcattaagaaataacctttatctggcaggagccctaagccacagctctaataatccaaccgttctcatttttctgtcttcctttctagtcctttcctataggaatatgcaaattaaaaaccaattaagttaattttaaaaatccaatgcatatcttgaaaccatacagagaagaatctcggttcactagggagatctctgtaggcttcactcatcaaaggtcaggcctgggtctcccacagcagtggggccagctatggagtttgcagggctggtgcaaaacaaaaatatgggcctcttgcacaaaatttactaagaatttcaaatggtggtggcagagccctgaaccccgcttgatcacatgcctgtgccactgcgtctgcggtgttctgaagttgtcctggaaagggctctgacctttgcccttccatcttctgtgtgccatggctgtccagcctccaggttcatggcctatcacaagcccctgaaaaactcacaggattacacagaagctctgcgggcagctcgagagctggcagccaacatcactgctgacctgcggaaagtgcctggaacagacccggcttttgaggtcttcccctacacgtgaggacctgagtggctgggctggagggaggtggggtatggttgctggagactggaggttagggtggagggcttgcaaggagttgcatgagatgaggaccagttttaggtcaggaggctctggctgcagccttgggcctatttcttaggctggtttgtaccccaatataagcctgcctgaccctcagcattctccttctgaagtggggtgtcccacccaccatgagggccccagaggcctgagcctgtgaccatgctctgtgctctggcaggatcaccaatgtgttttatgagcagtacctgaccatcctccctgaggggctcttcatgctcagcctctgccttgtgcccaccttcgctgtctcctgcctcctgctgggcctggacctgcgctccggcctcctcaacctgctctccattgtcatgatcctcgtggacactgtcggcttcatggccctgtggggcatcagttacaatgctgtgtccctcatcaacctggtctcggtaacccagcagacacaggcaccagggggcctctggaggggtggttggggatccagcctcatagaatactcctagttcttttttgtttctttttttagaggcagggtcttgctctgttgctcaggcttgagggcagtgacatgatcacagctcactgtagcctcgaacccttgggctcaagcgatcctcctacctcagcctccaaagtagccaggactacaggcacgtgccactgcgtccagctaatattttaatttttgttgtagagacagggtctcactttgttgcccaggctggtctcaaactcctgggctcaagtgatcctctcacctcggcctcccaaagtgttgggattataggcatgagccactgcacccggccaaatactcccagttctgtctagaatctagatgcctgccccacgctggtcctggtggaggcctcatctccctagttccttccccacctctgcctttcttggcttatgccccctctctgcccataggcggtgggcatgtctgtggagtttgtgtcccacattacccgctcctttgccatcagcaccaagcccacctggctggagagggccaaagaggccaccatctctatgggaagtgcggtgagtggagaggagtgggccaccctgtgccccactcgacaccctgtgccctgcctgatgccctgtgccctgcctgatgccctgtgccctgcctgacacctggctctgaaccccccaggtgtttgcaggtgtggccatgaccaacctgcctggcatccttgtcctgggcctcgccaaggcccagctcattcagatcttcttcttccgcctcaacctcctgatcactctgctgggcctgctgcatggcttggtcttcctgcccgtcatcctcagctacgtgggtgagtgcccaggcctgttcctaccagactgtcatgattatgctgacgacaacagtaacagtgcatgctcaccacaaaagctcaggaagtgcaaacgagccatgggcagatgtcagaagccaggactatgaccatgtggcaattctgtcttggaagctactattattcatttaatgtgctgtgaacatctttttttgtcagctatgtatgtctcaaacaacgtttctgtggccctgtacactgtggatcttcactgcactgctgttggacttttaagcatgcccttcagcaagaaatatattttacacagagaggtgacatgcacgggcacacatagacatgcctgcctaaaacaaatgcttcactaaataatattaatacttcctttatacatgtgaagcattctgatattgctggttccattctattattattattaatattttttggagacagggtcttgctctgacacccaggctggagtgcagtagcatgatcacagctcactgccaccttgacttcccaggctcaagtgatcctcccacctcagcctcccgagtagctgggaccacaggtgcacaccaccatgcccagctaattttttattttttgtagagatggggtctccctatgttgcccaggctggtctcaaactcctgagctcaagtgatccaccatggccttccacagtgctaggattacaggtgtgagccactgcgcttggcttttattttactttaaatttgttatttattttattttactttacattattttatttttattttttgagatggagtctcgctctgttgcccaggctggagtgcagtggtatgatctcagctccctgcaacctctgcctcccaagttcaagccattctcctgctttagcctcccaagtagctgggattacaggtgcgcaccaccacgcctggccaatttatttatttattttttatttttagtagagacggggtttcaccatgttgggcaggctggtctcgaactcctgacctcaggtgatccaaccgccaaggcctcccaaagtgctgggattacaggcgtgagccactgtgcccagccctatcattaatttgtttttaattattttaattatttttatttttattatttttagacagagtctctctctgttgcccaggctggagtgcagtggcgcaatctcagctcactgcaacctctgcctcctgggttcaagcgattctcctgtctcagcctctcgagtagctgggatatcggtgtatgccaccatacctggctaatttttgtatttttattggagacaggtttcaccatgttggtcaggctggtctcgaactcctgtggcctcaggtgatccatctgccttggcctcccaaagtgcagggattacaggcgtgggccaccgcacccggtctcattaatattttgaaatgctggccaggagtggtggctcatgtttgtaatcctagcactttgggaggctgaggcacatggaagctcaaattgagcctcccaggatgaaggtgtttctggctctcagggtgggcaagctgggaggagttcaattttacctcccaccagatggtaataatattattagaggacatttatagaggggtgtgtttgtgcatcaacatatgtgtctgtaattctcttactacccccgaggcaggtattattatccttcccattttacagatgagggaactgagacacctgccccaggttacagacttggtcaaaggtagtaggggttggagcccacacagctctgtggttcctaaccatgtctcttgtggggactccctgaccctcttggaaggagtagagtgtgtgcgctgggggtggtggatgagacataagagaggggcaaggaggagcagtcgtggggtgtgcttggacaaaggatatccagggccttggagctgcaggtggtggctattccttggaggttcccaaaatgcttgggggatggagggaccaggacatccctgaagcttgggctgtgaacatagtgaccctggaaggcacatggcacagatcccccctgggacccttcctgccctgggtttgttgtacagaaccaggaatagcttctcacctgtgtcccctgcccacctctctgactgtggttctctgtctctccgcagggcctgacgttaacccggctctggcactggagcagaagcgggctgaggaggcggtggcagcagtcatggtggcctcttgcccaaatcacccctcccgagtctccacagctgacaacatctatgtcaaccacagctttgaaggttctatcaaaggtgctggtgccatcagcaacttcttgcccaacaatgggcggcagttctgatacagccagaggccctgtctaggctctatggccctgaaccaaagggttatggggatcttccttgtgactgccccttgacacacgccctcctcaaatcctaggggaggccattcccatgagactgcctgtcactggaggatggcctgctcttgaggtatccaggcagcaccactgatggctcctctgctcccatagtgggtccccagtttccaagtcacctaggccttgggcagtgcctcctcctgggcctgggtctggaagttggcaggaacagacacactccatgtttgtcccacactcactcactttcctaggagcccacttctcatccaacttttcccttctcagttcctctctcgaaagtcttaattctgtgtcagtaagtctttaacacgtagcagtgtccctgagaacacagacaatgaccactaccctgggtgtgatatcacaggaggccagagagaggcaaaggctcaggccaagagccaacgctgtgggaggccggtcggcagccactccctccagggcgcacctgcaggtctgccatccacggccttttctggcaagagaagggcccaggaaggatgctctcataaggcccaggaaggatgctctcataagcaccttggtcatggattagcccctcctggaaaatggtgttgggtttggtctccagctccaatacttattaaggctgttgctgccagtcaaggccacccaggagtctgaaggctgggagctcttggggctgggctggtcctcccatcttcacctcgggcctggatcccaggcctcaaaccagcccaacccgagcttttggacagctctccagaagcatgaactgcagtggagatgaagatcctggctctgtgctgtgcacataggtgtttaataaacatttgttggcagaaatggtgttttatgtcacatgtcctaccctggcttcctcctctcggtttaagataatttttgtgaatgacacaaataatacatgtgtgggagagtgatttgtggagatactagtctgtgttttgttctatttctcctccctcttttcaagaaagtagccaggccattgtgtgctcatgccttacaagggcctttgaggagtgggagtaatttctcttcaaactgggagggcacagagcctgagagtcagtcaggagtaggatgtgcagcccctccttttctggaagagactgtgaagtaggcaacacctggaggagctacaggagaaccacggtgcattcaaggagggaagaacccaccgtacaaacaaccagctcccaggagggccccaggccagggcagtgggtggaaatgtcaaggaacattccagatcccctcgagtctttctgccccatgctgggtccagcccttgtttggctgaggggctgctgttgctttgaggctcagagggactgtcagcatgtaaagggaagacaagcaaaaaggggtggaaaggagctggcgtttctggagcctactatctacttttgggtcctcataagagccccatgtgccagcatcattagcccacctttgggagggttgctggctgaccatgatggacaggaggtttggtgaagggacagctacgagggaatagaggctgaggagaaatcgcacaattcaccctgttaaaaactccacaggtgcagaataaacagatagatttgaggaacaaaatagcttttgacagcagacatttcaaatcagaggaaagggtagatccttcagtaaacggtgtgagagtagtgagcaaattatttggatcaaaataaagttatatctatacttcacacaatacacaaaataaaagtacagacagattaaagcactaaacacaaaaatgaaactatacaactatcggaaggaaacacagaagagtatgttataatcttggagggggaaaagtttcctaagcacaaagtccagaagccataaaggtaaacactaaggtatgaccatataataatggaaaacatctgaaaacacacaaaaaattaaagaaagttgaaagacacatatgagctcagaaaaatagttgcaacatatttaacagcaaataaaatcaagaaaacacaaagagtgccaatagtgctcctgcaaacatggtgaacactcctaaaacccactggactttctgtaagaagtgtgggaagcaccagccccacagagtgacacaggacacatttccctgtatgcctagggaaagccatgttatgacaggaagcagaggggctatggtggggagactaagccaattttccagaaaaaggctaaaactacaaagaagattgtgctaagttttgagtgcatgaagcccaactgcagatctaagagaatgctggctattaagagatacaagcattttgaactgggaggaggtaagaagagcaagggccaagtgatccagttctaagtgtcatcttttgttttattatgaagacaataaaatattgagtttatgtttaaaaaaaaaaagaatatacaaagagagtccaggtacggtggctcatgcctgtaatcccagcactttgggaggctgaggcaggagaattgcttgaggccaggagttcaagaccagcctaggcaacatagcgagatactgtctctacaaaaagtttaaaagttagccaggctagctatttggaaggctgaggtgggaggattgtttcagctcgagtttgaggctgcagtgagctatgatggcaccactgtactccagcctgagtgaaagagtgagcttctgtctcaacaaaaaaaaaaaaaaaaaagaatatacaaagagaggaaggagtgcaggggggaggtctgggttatgtggctaaccttcccattagaaacaagacattctagctaaaataaatcttagccgtgtgtgtgtgtgtatgtgtctgtgtgtgtgtatgatgcatacaagtttagggtgttttaaccttcttgataaattgagacttttatagtttgaaatgactataaaaatatccctttttatctctagtatttatttttgtctgtttaagagatggggttctcactttgttgcccaggctggtcttgaatacttggcctcaagggatcctcctacctcagcctcccaagtacctggaattacaggtatgagccaccatgccagtcctatctgtagtatttgttcaactgtataatgttattatacacacacacacacacacacacacacacacacagacacacacacacatataaaataacatacggttgaacaaattttatacttaatagtcaaacattgaaaccctttcccctgagattgggaatgagacaaagttgcccacttttacccaacattgcactggaggtcttagccattgtaataaggcaagaaaaagaaactaagtttataaggattagaaataaataaaattgacatcattcacagataacataaatatgtataaaaaagattcagtctgggtgcagtggctcatgcctgtaaccccagcaatttctgaggccaaggcaggaggatcacttgaggccaggagttcaagacatagcaagaccccacctctacaaaaaaaaattttttttaaagatccaaaagaatctatatataaactattggaattactctaacaaaaggtggtcaagaaaactatgaaaaataataactttgtattttaatttgtataatattgagagaaattaactgtcaaaagaaatggaggaatataccatgaattgagggctctatactacagagatgtcaattctcttcaaattaattactagtttcactgtaatttcaataataaccccagaaaattttttgtggaaactgataagctgattcaaaaattcatatagaaccacaaaagatgaaaattcacgaaagcaatcttgaagaaaaacaaagtcagagaacttacactactagaaatcaagataatataaatatatagaaataaagatagtgagattttggcacaaggaagaacaaatagaaaaatggaaagaatagaaagtccagaaacagatgatacccacaaggacacatgatttatgatggaggaggcatgcagagcattgggtaaaggaggtttttcaatgtaggatgctgacctagttgggtatccacacagaaagaaatgaatcatgaccctctcccccaagatacacaaaaatcagttcctgatagattgtcaatctaaatgtgaaagataaaatgatagagttctaaaaggtaacataaaagagtatccccaagactgaaataggaaaaacttttcttaggaaacaaaagccttacttatagagaaaaagattgataaattgaactgtattggaataaaaaaaaacttctgttcttcaaaagacatccttaggaaagataaaattcaaaccatagagaggaaaagatatttgcacatatctgaaatacacacatatctgagaaagggcctgtgcttagaatgcataaaaaatctcctacaactcagcaagaaaaagacagacaaccaaaagaaaagctaggctggctactcaaataagcaaatggccaatacaagttcctcaattttgtcagtcaccagagcaaggctgagtaaaagcacagtgagagttcttcctcttctcttccctcacaatttggcctacaggccatggggtaaggtggggccaggcagcacatgtggggtgtcagaatccaggtggtgtggggagcgtttccacattggatctgagggaggagaggagggcattccacacagaataggaactaca

SEQ ID NO: 2 - DNA, 12 bp, Homo sapiens
ggaggctgcctt

SEQ ID NO: 3 - DNA, 12 bp, Homo sapiens
ggaggatgcctt

SEQ ID NO: 4 - DNA, 23 bp, Homo sapiens
ctaatagcgtggtctctccccta

SEQ ID NO: 5 - DNA, 22 bp, Homo sapiens
atccctcatgtgtccagagact

SEQ ID NO: 6 - DNA, 18 bp, Homo sapiens
tgtaaaacgacggccagt

SEQ ID NO: 7 - DNA, 18 bp, Homo sapiens
caggaaacagctatgacc

SEQ ID NO: 8 - DNA, 25 bp, Homo sapiens
agaatggtaaacattgtactctgac

SEQ ID NO: 9 - DNA, 22 bp, Homo sapiens
ttcatatgtttcttcccatggg

SEQ ID NO: 10 - DNA, 25 bp, Homo sapiens
gagcaaaggagagtcttccactatc

SEQ ID NO: 11 - DNA, 22 bp, Homo sapiens
caagggctgaacacacattaag

SEQ ID NO: 12 - DNA, 23 bp, Homo sapiens
tgtcttgagaacttaggggtcag

SEQ ID NO: 13 - DNA, 22 bp, Homo sapiens
cactgtcatccctagcaactgt

SEQ ID NO: 14 - DNA, 25 bp, Homo sapiens
gactttcctaagctgcaggtctatc

SEQ ID NO: 15 - DNA, 23 bp, Homo sapiens
gttcacaaaattgtcagagcagg

SEQ ID NO: 16 - DNA, 24 bp, Homo sapiens
ctgctctgacaattttgtgaacct

SEQ ID NO: 17 - DNA, 23 bp, Homo sapiens
agacagagcagaggatgatgatg

SEQ ID NO: 18 - DNA, 26 bp, Homo sapiens
acccagagctgtctggaagcctcatg

SEQ ID NO: 19 - DNA, 22 bp, Homo sapiens
ccattgcctgtgtctccctgga

SEQ ID NO: 20 - DNA, 22 bp, Homo sapiens
ctcgactccaccttctacctgg

SEQ ID NO: 21 - DNA, 25 bp, Homo sapiens
cagagagtcatacctgtagctggac

SEQ ID NO: 22 - DNA, 22 bp, Homo sapiens
aagctttccatgaccagcattt

SEQ ID NO: 23 - DNA, 23 bp, Homo sapiens
agccgtaggaatagctacctctg

SEQ ID NO: 24 - DNA, 25 bp, Homo sapiens
agtactccatactccagagcaaatg

SEQ ID NO: 25 - DNA, 25 bp, Homo sapiens
gtattgaggttagatttggaaccct

SEQ ID NO: 26 - DNA, 25 bp, Homo sapiens
tcttgctttaagtctgacagaggag

SEQ ID NO: 27 - DNA, 24 bp, Homo sapiens
gttcctgctatttccaagagagag

SEQ ID NO: 28 - DNA, 25 bp, Homo sapiens
ggtcctaaatagctaaatggcctaa

SEQ ID NO: 29 - DNA, 25 bp, Homo sapiens
ccacagtgcctgagtaacactacta

SEQ ID NO: 30 - DNA, 25 bp, Homo sapiens
tttacagacaggaaaactgaggttc

SEQ ID NO: 31 - DNA, 24 bp, Homo sapiens
ctgcatttaggccatttagctatt

SEQ ID NO: 32 - DNA, 25 bp, Homo sapiens
agagaagtggggtgtaggaggtaag

SEQ ID NO: 33 - DNA, 25 bp, Homo sapiens
tataatcgcaggtgaggctataaga

SEQ ID NO: 34 - DNA, 22 bp, Homo sapiens
gtcttgggtcagttcctgtgtc

SEQ ID NO: 35 - DNA, 22 bp, Homo sapiens
agaggtattaccctttggggca

SEQ ID NO: 36 - DNA, 25 bp, Homo sapiens
cttttctcttctcttttccctccta

SEQ ID NO: 37 - DNA, 25 bp, Homo sapiens
gctcacacctgtaatctcaacattt

SEQ ID NO: 38 - DNA, 23 bp, Homo sapiens
atgctcaaggaagatggagtagg

SEQ ID NO: 39 - DNA, 24 bp, Homo sapiens
gtgtcgatgaacagaaagagtctg

SEQ ID NO: 40 - DNA, 24 bp, Homo sapiens
agtctctgatgattcaggaaggtc

SEQ ID NO: 41 - DNA, 24 bp, Homo sapiens
aatattactctcctggcacaatgc

SEQ ID NO: 42 - DNA, 25 bp, Homo sapiens
cattccatggtaaggataaatcaga

SEQ ID NO: 43 - DNA, 22 bp, Homo sapiens
acatctgcaggaggaagtcaag

SEQ ID NO: 44 - DNA, 25 bp, Homo sapiens
cattccatggtaaggataaatcaga

SEQ ID NO: 45 - DNA, 24 bp, Homo sapiens
aatattactctcctggcacaatgc

SEQ ID NO: 46 - DNA, 25 bp, Homo sapiens
taagcagttgaaaatctgcatgtaa

SEQ ID NO: 47 - DNA, 23 bp, Homo sapiens
ctcttcctcagcctactcaacct

SEQ ID NO: 48 - DNA, 22 bp, Homo sapiens
agtgatccttgacttcctcctg

SEQ ID NO: 49 - DNA, 25 bp, Homo sapiens
tgaaaccccatctctattaaaaaca

SEQ ID NO: 50 - DNA, 24 bp, Homo sapiens
aagtctgctcaactccagaatgtt

SEQ ID NO: 51 - DNA, 24 bp, Homo sapiens
ctgttgtgctgttcatacacgaat

SEQ ID NO: 52 - DNA, 25 bp, Homo sapiens
tataaatgagaggtcgacaggagtt

SEQ ID NO: 53 - DNA, 25 bp, Homo sapiens
acaaatttaagtcagtcagggtgtc

SEQ ID NO: 54 - DNA, 24 bp, Homo sapiens
gaagagaatccagggataagtgag

SEQ ID NO: 55 - DNA, 25 bp, Homo sapiens
aaatttaagtcagtcagggtgtcat

SEQ ID NO: 56 - DNA, 25 bp, Homo sapiens
cacagacaacaaagtctgagacaca

SEQ ID NO: 57 - DNA, 25 bp, Homo sapiens
aaatgtccccaacagaaaaataaac

SEQ ID NO: 58 - DNA, 25 bp, Homo sapiens
agaggtgcagaattgttcattactc

SEQ ID NO: 59 - DNA, 25 bp, Homo sapiens
atgtgtctcagactttgttgtctgt

SEQ ID NO: 60 - DNA, 25 bp, Homo sapiens
aactttacccaacaaacagtgactc

SEQ ID NO: 61 - DNA, 25 bp, Homo sapiens
gcgaaaccctgtctctactaaaagt

SEQ ID NO: 62 - DNA, 25 bp, Homo sapiens
actgtactttgggtgactttatgga

SEQ ID NO: 63 - DNA, 25 bp, Homo sapiens
gagtcactgtttgttgggtaaagtt

SEQ ID NO: 64 - DNA, 25 bp, Homo sapiens
ttctatgagtttgaccactctaggc

SEQ ID NO: 65 - DNA, 25 bp, Homo sapiens
attaaacacacacacacacacacac

SEQ ID NO: 66 - DNA, 25 bp, Homo sapiens
tttttctgttcttccactttcaatc

SEQ ID NO: 67 - DNA, 25 bp, Homo sapiens
aaaagagagtagtaggaccaggcat

SEQ ID NO: 68 - DNA, 25 bp, Homo sapiens
tacctttgccagggatttatttatt

SEQ ID NO: 69 - DNA, 25 bp, Homo sapiens
tgaaggaattcgttatcactagacc

SEQ ID NO: 70 - DNA, 25 bp, Homo sapiens
cttgagtagctgggactacaggtat

SEQ ID NO: 71 - DNA, 25 bp, Homo sapiens
attcaaaagcagtcagaagaaagaa

SEQ ID NO: 72 - DNA, 25 bp, Homo sapiens
tcctcattgatatttccattttgtt

SEQ ID NO: 73 - DNA, 25 bp, Homo sapiens
aaaaatgcagtctcaaaaatacctg

SEQ ID NO: 74 - DNA, 25 bp, Homo sapiens
caaaggcacagagttaatgtcttct

SEQ ID NO: 75 - DNA, 25 bp, Homo sapiens
acacttgtaatttcagaactttggg

SEQ ID NO: 76 - DNA, 22 bp, Homo sapiens
ctgatgttctatccctgtcctg

SEQ ID NO: 77 - DNA, 22 bp, Homo sapiens
cacctacaaatgccactgcttt

SEQ ID NO: 78 - DNA, 25 bp, Homo sapiens
tgcatgtacctctgtgtacctctaa

SEQ ID NO: 79 - DNA, 24 bp, Homo sapiens
acagggatagaacatcaggaagag

SEQ ID NO: 80 - DNA, 24 bp, Homo sapiens
ccacagtttctatagccaagagga

SEQ ID NO: 81 - DNA, 25 bp, Homo sapiens
agtcaagttcacagaggtgctgtat

SEQ ID NO: 82 - DNA, 25 bp, Homo sapiens
gagcagttccataagtatcttccct

SEQ ID NO: 83 - DNA, 25 bp, Homo sapiens
gaatcaattccacaaacttagcact

SEQ ID NO: 84 - DNA, 25 bp, Homo sapiens
acctctacctcctggattcaagtaa

SEQ ID NO: 85 - DNA, 22 bp, Homo sapiens
atcttggctcactgcaacttct

SEQ ID NO: 86 - DNA, 25 bp, Homo sapiens
acctctacctcctggattcaagtaa

SEQ ID NO: 87 - DNA, 25 bp, Homo sapiens
cttgtttttgttttcgagacagagt

SEQ ID NO: 88 - DNA, 25 bp, Homo sapiens
tactaagaatttcaaatggtggtgg

SEQ ID NO: 89 - DNA, 25 bp, Homo sapiens
ggtacaaaccagcctaagaaatagg

SEQ ID NO: 90 - DNA, 22 bp, Homo sapiens
gttgctggagactggaggttag

SEQ ID NO: 91 - DNA, 25 bp, Homo sapiens
aactaggagtattctatgaggctgg

SEQ ID NO: 92 - DNA, 25 bp, Homo sapiens
aaagtgttgggattataggcatgag

SEQ ID NO: 93 - DNA, 24 bp, Homo sapiens
aagaagaagatctgaatgagctgg

SEQ ID NO: 94 - DNA, 24 bp, Homo sapiens
atcagttacaatgctgtgtccctc

SEQ ID NO: 95 - DNA, 22 bp, Homo sapiens
gggaaggaactagggagatgag

SEQ ID NO: 96 - DNA, 22 bp, Homo sapiens
gtggagtttgtgtcccacatta

SEQ ID NO: 97 - DNA, 25 bp, Homo sapiens
atagtagcttccaagacagaattgc

SEQ ID NO: 98 - DNA, 23 bp, Homo sapiens
tatggggatcttccttgtgactg

SEQ ID NO: 99 - DNA, 22 bp, Homo sapiens
cttatgagagcatccttcctgg

SEQ ID NO: 100 - DNA, 22 bp, Homo sapiens
cttgggctgtgaacatagtgac

SEQ ID NO: 101 - DNA, 22 bp, Homo sapiens
ctccagtgacaggcagtctcat

SEQ ID NO: 102 - DNA, 25 bp, Homo sapiens
aagtctttaacacgtagcagtgtcc

SEQ ID NO: 103 - DNA, 25 bp, Homo sapiens
aaagagggaggagaaatagaacaaa

SEQ ID NO: 104 - DNA, 24 bp, Homo sapiens
cagtgggagtggtggatcattaac

SEQ ID NO: 105 - DNA, 19 bp, Homo sapiens
ctggcctgactgggttagg

SEQ ID NO: 106 - DNA, 16 bp, Homo sapiens
ccaatgaggctgagcc

SEQ ID NO: 107 - DNA, 16 bp, Homo sapiens
ccaatgaggccgagcc

SEQ ID NO: 108 - DNA, 14 bp, Homo sapiens
ggcctggcctggct

SEQ ID NO: 109 - DNA, 17 bp, Homo sapiens
cgccatcccaggtctgg

SEQ ID NO: 110 - DNA, 14 bp, Homo sapiens
ccgctgaccccttc

SEQ ID NO: 111 - DNA, 13 bp, Homo sapiens
cgctgaacccttc

SEQ ID NO: 112 - DNA, 20 bp, Homo sapiens
gcatcctgtcctgccatagc

SEQ ID NO: 113 - DNA, 19 bp, Homo sapiens
gcatctggcccaggtagaa

SEQ ID NO: 114 - DNA, 14 bp, Homo sapiens
ccctcgactccacc

SEQ ID NO: 115 - DNA, 14 bp, Homo sapiens
ccctggactccacc

SEQ ID NO: 116 - DNA, 17 bp, Homo sapiens
cccgtggagctgtggtc

SEQ ID NO: 117 - DNA, 22 bp, Homo sapiens
gaaatgctggtcatggaaagct

SEQ ID NO: 118 - DNA, 14 bp, Homo sapiens
cccccaacagccaa

SEQ ID NO: 119 - DNA, 13 bp, Homo sapiens
ccccagcagccaa

SEQ ID NO: 120 - DNA, 23 bp, Homo sapiens
ctgaccttacagaccctggaaag

SEQ ID NO: 121 - DNA, 24 bp, Homo sapiens
ccaatccagtggttctcaaagtgt

SEQ ID NO: 122 - DNA, 15 bp, Homo sapiens
cccttaggcgtcctg

SEQ ID NO: 123 - DNA, 15 bp, Homo sapiens
cccttaggcatcctg

SEQ ID NO: 124 - DNA, 20 bp, Homo sapiens
ctcgaggtgttgtggtgagt

SEQ ID NO: 125 - DNA, 18 bp, Homo sapiens
gcgaggtccccacctagt

SEQ ID NO: 126 - DNA, 16 bp, Homo sapiens
ctgctctcgtgtggtt

SEQ ID NO: 127 - DNA, 17 bp, Homo sapiens
cctgctctcatgtggtt

SEQ ID NO: 128 - DNA, 18 bp, Homo sapiens
tcttgaatgtttatattc

SEQ ID NO: 129 - DNA, 17 bp, Homo sapiens
gtaagaattccagaaga

SEQ ID NO: 130 - DNA, 19 bp, Homo sapiens
caaataatctcacttcccc

SEQ ID NO: 131 - DNA, 16 bp, Homo sapiens
ataatctcgcttcccc

SEQ ID NO: 132 - DNA, 23 bp, Homo sapiens
tgtgtgtaccttcgagagtgtga

SEQ ID NO: 133 - DNA, 21 bp, Homo sapiens
tgagctttggttcgctatgca

SEQ ID NO: 134 - DNA, 16 bp, Homo sapiens
taaagggctcaatcca

SEQ ID NO: 135 - DNA, 17 bp, Homo sapiens
ctaaagggctcactcca

SEQ ID NO: 136 - DNA, 21 bp, Homo sapiens
gagttccctgagcagtgagtt

SEQ ID NO: 137 - DNA, 25 bp, Homo sapiens
gacagggatagaacatcaggaagag

SEQ ID NO: 138 - DNA, 14 bp, Homo sapiens
ctggcccgccccaa

SEQ ID NO: 139 - DNA, 14 bp, Homo sapiens
ctggcccaccccaa

SEQ ID NO: 140 - DNA, 19 bp, Homo sapiens
cccaaaccccagcctactc

SEQ ID NO: 141 - DNA, 25 bp, Homo sapiens
gacagggatagaacatcaggaagag

SEQ ID NO: 142 - DNA, 19 bp, Homo sapiens
ctgtttgagtccctccagt

SEQ ID NO: 143 - DNA, 19 bp, Homo sapiens
ctgtttgagtccccccagt

SEQ ID NO: 144 - DNA, 19 bp, Homo sapiens
ggtcttcctgcccgtcatc

SEQ ID NO: 145 - DNA, 26 bp, Homo sapiens
agcataatcatgacagtctggtagga

SEQ ID NO: 146 - DNA, 16 bp, Homo sapiens
tcacccacgtagctga

SEQ ID NO: 147 - DNA, 16 bp, Homo sapiens
tcacccacatagctga

SEQ ID NO: 148 - DNA, 23 bp, Homo sapiens
tctgactgtggttctctgtctct

SEQ ID NO: 149 - DNA, 18 bp, Homo sapiens
ctcctcagcccgcttctg

SEQ ID NO: 150 - DNA, 15 bp, Homo sapiens
ccgggttaacgtcag

SEQ ID NO: 151 - DNA, 15 bp, Homo sapiens
ccgggttgacgtcag

SEQ ID NO: 152 - DNA, 18 bp, Homo sapiens
gcccaacccgagcttttg

SEQ ID NO: 153 - DNA, 23 bp, Homo sapiens
cacagagccaggatcttcatctc

SEQ ID NO: 154 - DNA, 16 bp, Homo sapiens
ccagaagcatgaactg

SEQ ID NO: 155 - DNA, 15 bp, Homo sapiens
cagaagcgtgaactg

(19) United States
(12) Patent Application Publication
(10) Pub. No.: US 2009/0192135 A1
(43) Pub. Date: Jul. 30, 2009

(54) Human Niemann Pick C1-Like 1 Gene (NPC1L1) Polymorphisms and Methods of Use Thereof

(75) Inventors: Jason Samuel Simon, Westfield, NJ (US); Maha Chabhar Karnoub, Doylestown, PA (US); Michael E. Severino, Westlake Village, CA (US); David James Devlin, New City, NY (US); Andrew Stewart Plump, Westfield, NJ (US); Eric E. Schadt, Kirkland, WA (US)

Correspondence Address: FOLEY AND LARDNER LLP;SUITE 500, 3000 K STREET NW, WASHINGTON, DC, 20007, US

(73) Assignees: SCHERING CORPORATION, Kenilworth, NJ (US); MERCK & CO., INC., Rahway, NJ (US); ROSETTA INPHARMATICS LLC, Seattle, WA (US)

(21) Appl. No.: 11/887,346

(22) PCT Filed: Mar. 28, 2006
(86) PCT No.: PCT/US2006/012727

§ 371 Date: Feb. 13, 2009

Related U.S. Application Data
(60) Provisional application No. 60/667,047, filed on Mar. 30, 2005.
(60) Provisional application No. 60/717,465, filed on Sep. 14, 2005.

(51) Int. Cl. A61K 31/397; C12Q 1/60; C12Q 1/68; C40B 30/04; C07H 21/04

(52) U.S. Cl. 514/210.02; 435/11; 435/6; 506/9; 536/23.5

(57) ABSTRACT

The present invention relates to the identification and use of single nucleotide polymorphisms and haplotypes in the Niemann Pick C1-Like 1 (NPC1L1) gene. In particular, methods are provided for correlating NPC1L1 polymorphisms and haplo-types with the responsiveness of a pharmaceutically active compound administered to a human subject. The invention further relates to a method for estimating the responsiveness of a pharmaceutically active compound administered to a human subject which method comprises determining at least one polymorphism in the NPC1L1 gene. The methods are based on determining polymorphisms in the NPC1L1 gene and correlating the responsiveness of a pharmaceutically active compound in the human by reference to one or more polymorphism in NPC1L1. The invention further relates to isolated nucleic acids comprising within their sequence the polymorphisms as defined herein, to nucleic acid primers and oligonucleotide probes capable of hybridizing to such nucleic acids and to a diagnostic kit comprising one or more of such primers and probes for detecting a polymorphism in the NPC1L1 gene.

This application claims priority to U.S. Provisional Patent Application Serial No. 06/667,047 filed on Mar. 30, 2005, and U.S. Provisional Patent Application Ser. No. 60/717,465 filed on Sep. 14, 2005, each of which is incorporated by reference herein in its entirety.

### BACKGROUND OF THE INVENTION

Pharmacogenetics is the study of the role of genetics in the variation in drug metabolism and drug response. Pharmacogenetics helps to identify patients most suited to therapy with a particular pharmaceutical agent. This approach can be used in pharmaceutical research to assist the drug selection process and can help to select patient for enrollment into clinical trials. Details on pharmacogenetics and other uses of polymorphism detection can be found in Linder et al., (1997) Clinical Chemistry, 43:254; Marshall (1997) Nature Biotechnology, 15:1249; PCT Patent Application WO 97/40462, Spectra Biomedical; and Schafer et al., (1998) Nature Biotechnology 16: 33.

Moreover, polymorphisms are implicated in over 2000 human pathological syndromes resulting from DNA insertions, deletions, duplications and nucleotide substitutions. Finding genetic polymorphisms in individuals and following these variations in families provides a means to confirm clinical diagnoses and to diagnose both predispositions and disease states in carriers, as well as preclinical and subclinical affected individuals. Further, genetic polymorphisms may be used to identify individuals who may be more responsive to one therapeutic treatment over another.

Polymorphisms associated with phenotypes are difficult to identify. Because multiple alleles within genes are common, one must distinguish disease-related alleles from neutral (non-disease-related) polymorphisms. Most alleles are neutral polymorphisms that produce indistinguishable, normally active gene products or express normally variable characteristics like eye color. In contrast, some polymorphic alleles are associated with clinical diseases such as sickle cell anemia. Moreover, the structure of disease-related polymorphisms are highly variable and may result from a single point mutation as occurs in sickle cell anemia, or from the expansion of nucleotide repeats as occurs in fragile X syndrome and Huntington's chorea.

A factor leading to development of vascular disease, a leading cause of death in industrialized nations, is elevated serum cholesterol. It is estimated that 19% of Americans between the ages of 20 and 74 years of age have high serum cholesterol. The most prevalent form of vascular disease is arteriosclerosis, a condition associated with the thickening and hardening of the arterial wall. Arteriosclerosis of the large vessels is referred to as atherosclerosis. Atherosclerosis is the predominant underlying factor in vascular disorders such as coronary artery disease, aortic aneurysm, arterial disease of the lower extremities and cerebrovascular disease.

Cholesteryl esters are a major component of atherosclerotic lesions and the major storage form of cholesterol in arterial wall cells. Formation of cholesteryl esters is also a step in the intestinal absorption of dietary cholesterol. Thus, inhibition of cholesteryl ester formation and reduction of serum cholesterol can inhibit the progression of atherosclerotic lesion formation, decrease the accumulation of cholesteryl esters in the arterial wall, and block the intestinal absorption of dietary cholesterol.

The regulation of whole-body cholesterol homeostasis in mammals and animals involves the regulation of intestinal cholesterol absorption, cellular cholesterol trafficking, dietary cholesterol and modulation of cholesterol biosynthesis, bile acid biosynthesis, steroid biosynthesis and the catabolism of the cholesterol-containing plasma lipoproteins. Regulation of intestinal cholesterol absorption has proven to be an effective means by which to regulate serum cholesterol levels. For example, a cholesterol absorption inhibitor, ezetimibe, has been shown to be effective in this regard (Kropp et al., (2002) Int. J. Clin. Pract. 57:363-8).

Recently the Niemann Pick C1-Like 1 (NPC1L1) gene was identified as encoding the protein through which the cholesterol drug ezetimibe (ZETIA®) acts to block intestinal absorption of cholesterol (Altmann, et al., (2004) Science, 303: 1201-04; and Davis, et al., (2004) J. Biol. Chem., 279:33586-92). Ezetimibe is effective in reducing LDL-Cholesterol (LDL-C) both in monotherapy and in combination with statins, such as simvastatin (ZOCOR®).

NPC1L1 is an N-glycosylated protein comprising a four amino acid motif that serves as a trans-golgi network to plasma membrane transport signal (see Bos, et al., (1993) EMBO J. 12:2219-28; Humphrey, et al., (1993) J. Cell. Biol. 120:1123-35; Ponnambalam, et al., (1994) J. Cell. Biol. 125:253-268 and Rothman, et al., (1996) Science 272:227-34). The NPC1L1 protein has limited tissue distribution and gastrointestinal abundance. Also, the human NPC1L1 promoter region includes a Sterol Regulated Element Binding Protein 1 (SREBP1) binding consensus sequence (Athanikar, et al., (1998) Proc. Natl. Acad. Sci. USA 95:4935-40; Ericsson, et al., (1996) Proc. Natl. Acad. Sci. 93:945-50; Metherall, et al., (1989) J. Biol. Chem. 264:15634-41; Smith, et al., (1990) J. Biol. Chem. 265:2306-10; Bennett, et al., (1999) J. Biol. Chem. 274:13025-32 and Brown, et al., (1997) Cell 89:331-40). NPC1L1 has 42% amino acid sequence homology to human NPC1 (Genbank Accession No. AF002020), a receptor responsible for Niemann-Pick C1 disease (Carstea, et al., (1997) Science 277:228-31).

Niemann-Pick Type C disease is a rare genetic disorder in humans which results in accumulation of low density lipoprotein (LDL)-derived unesterified cholesterol in lysosomes (Pentchev, et al., (1994) Biochim. Biophys. Acta. 1225: 235-43 and Vanier, et al., (1991) Biochim. Biophys. Acta. 1096:328-37). In addition, cholesterol accumulates in the trans-golgi network of cells lacking NPC1, and relocation of cholesterol, to and from the plasma membrane, is delayed. NPC1 and NPC1L1 each possess 13 transmembrane spanning segments as well as a sterol-sensing domain (SSD). Several other proteins, including HMG-CoA Reductase (HMG-R), Patched (PTC) and Sterol Regulatory Element Binding Protein Cleavage-Activation Protein (SCAP), include an SSD which is involved in sensing cholesterol levels possibly by a mechanism which involves direct cholesterol binding (Gil, et al., (1985) Cell 41:249-58; Kumagai, et al., (1995) J. Biol. Chem. 270:19107-13 and Hua, et al., (1996) Cell 87:415-26). The NPC1L1 protein has many properties consistent with a role in cholesterol transport including a high degree of homology to Niemann Pick type C1 (NPC1) as well as a putative sterol sensing domain (SSD) with homology to those of 3-hydroxy 3-methylglutaryl coenzyme A reductase (HMGR) and sterol regulatory element-binding proteins cleavage-activating protein (SCAP). However, NPC1 and NPC1L1 differ significantly in their putative targeting signals, suggesting different cellular localization (Davis, et al., (2004) J. Biol. Chem., 279:33586-92).

NPC1L1 is expressed at relatively low levels, but is generally expressed over a number of human tissues and cell lines and is enriched in the small intestine, where it is restricted to the enterocyte as demonstrated by in situ hybridization (Altmann et al., (2004) Science, 303:1201-04). The highest levels of NPC1L1 expression have been observed in the proximal jejunum, which is also the primary site of cholesterol absorption. Furthermore, recent studies have shown that NPC1L1-null (−/−) mice exhibit a 69% reduction in dietary cholesterol absorption as compared to wild-type which is not rescued by dietary supplementation with exogenous bile salts or further reduced following treatment with the cholesterol absorption inhibitor, ezetimibe (Altmann et al., (2004) Science, 303:1201-04). Thus, NPC1L1 plays an important role in intestinal cholesterol absorption and appears to reside within an ezetimibe-sensitive pathway.

Several clinical studies have demonstrated the efficacy of ezetimibe monotherapy in lowering LDL-C (Knopp, et al., (2003) Int. J. Clin. Pract. 57:363-8; Knopp, et al., (2003) Eur. Heart J. 24:72941). Mean reductions of 18-19% are observed with ezetimibe 10 mg/day monotherapy (Ezzet, et al., (2001) J. Clin. Pharmaco., 41:943-9), and similar reductions are seen with ezetimibe co-administration or add-on therapy to statins (Davidson, et al., (2002) J. Am. Coll. Cardiol. 40:2125-34; Pearson, et al., (2005) Mayo Clinic Proceedings, 80:587-95). Consistent with its pharmacological mechanism of action, studies in humans suggest that the ezetimibe mediated decrease in plasma LDL-C results from the inhibition of intestinal cholesterol absorption (Sudhop and von Bergmann (2002) Drugs, 62:233347). Interestingly, significant inter-individual variability has been observed for rates of intestinal absorption and LDL-C reductions at both baseline and post ezetimibe treatment.

Because of the important role of cholesterol management in human health, genetic factors, such as polymorphisms and haplotypes that are associated with one or more drug responses have utility in the making of health management decisions. It has now been found that polymorphisms and haplotypes in the NPC1L1 gene can be used to estimate the responsiveness of a pharmaceutically active compound, e.g., a NPC1L1 antagonist, administered to a human subject.

The human NPC1L1 gene maps to chromosome 7p13, spans approximately 29 Kb, and contains 20 exons (Davis, et al., (2004) J. Biol. Chem. 279: 33586-92). A reference sequence for the human NPC1L1 gene is listed in SEQ ID NO: 1. A number of single nucleotide polymorphisms (SNPs) in the human NPC1L1 gene have been reported (see, e.g., the Single Nucleotide Polymorphism database (dbSNP) maintained by the National Center for Biotechnology Information (NCBI)). However, only a few of these SNPs have a reported minor allele frequency (MAF) of greater than 10%.

A recent report described a study in which the exons and intron-exon boundaries of the NPC1L1 gene of eight nonresponders to ezetimibe (i.e., LDL cholesterol change ranged from a 6% decrease to a 10% increase) and six ezetimibe responders were examined for polymorphisms (Wang J. et al., (February 2005) Clin. Genet. 67(2): 175-177). The report states that one of the eight non-responders was a compound heterozygote for two rare NPC1L1 polymorphisms that were absent in the six control subjects, but does not state whether either polymorphism was detected in any of the other non-responders. One polymorphism was G219T in exon 2, which results in a substitution of leucine for valine at amino acid position 55 (V55L); the other polymorphism was T3754A in exon 18, which results in a substitution of asparagine for isoleucine at amino acid position 1233 (II233N). The authors stated that one of many possible explanations for this data was a possible relationship between ezetimibe response and NPC1L1 variation. However, the authors also reported that the minor allele frequencies of thirteen other NPC1L1 polymorphisms were not statistically significant different between responders and non-responders, including six SNPs seen only in non-responders. Thus, the skilled artisan would have no expectation from this reference that correlations between increased response to ezetimibe and any common allele (>5% frequency) of the NPC1L1 gene could be successfully identified.

### SUMMARY OF THE INVENTION

The present invention relates to SNPs and haplotypes associated to an increased response to NPC1L1 antagonists. Patients having the inventive polymorphisms exhibit a higher than average response to NPC1L1 antagonists as indicated, for example, by an increased average lowering of serum low density lipoprotein cholesterol levels as compared to individuals not having the inventive polymorphisms. In addition, a NPC1L1 SNP was identified as associated with an increased risk of elevated LDL-C. The SNPs and haplotypes associated with increased LDL-C lowering were identified by examining the genotype of patients given a statin compound versus patients given a statin plus ezetimibe. The tested patient population was not meeting the recommended level of LDL-C through a statin alone. Ezetimibe resulted in a LDL-C reduction in all of the treated patients, however, the LDL-C lowering due to ezetimibe varied in different groups of patients. Through genotypic analysis of the different patients, SNPs and haplotypes associated with an increased response to ezetimibe were identified.

The identified SNPs and haplotypes associated with an increased LDL-C lowering due to an NPC1L1 antagonists are particularly useful in providing an indication as to a patient's (i.e., human) degree of responsiveness to the compound. The indication can be used by the physician to help predict the outcome of a particular treatment. In addition, the phenotypic effect of the NPC1L1 markers described herein support using these markers in a variety of methods and products, including, but not limited to: diagnostic methods and kits; pharmacogenetic treatment methods, which involve tailoring a patient's drug therapy based on whether the patient tests positive or negative for an NPC1L1 marker associated with response to an NPC1L1 antagonist; drug development and marketing, and pharmacogenetic drug products.

In one aspect the present invention provides a method of correlating single nucleotide polymorphisms and haplotypes in the NPC1L1 gene with an activity of a pharmaceutically active compound administered to a human subject. The method comprises associating a single nucleotide polymorphism or haplotype in the NPC1L1 gene of the human subject with the status of the human subject to which the pharmaceutically active compound was administered by reference to the single nucleotide polymorphism or haplotype in the NPC1L1 gene. In some embodiments, the status of the subject is determined by measuring a plasma component level, such as, for example, low density lipoprotein cholesterol (LDL-C), total cholesterol, non-high density lipoprotein cholesterol (non-HDL-C), and apolipoprotein B, before and after administration of the compound. In a particular embodiment, the plasma component is LDL-C and the compound activity is the lowering of LDL-C in the subject as compared to the level of plasma LDL-C in the subject prior to administration of the compound. In other embodiments, the single nucleotide polymorphism is selected from the group consisting of g.−133A>G, g.−18C>A, g.1679C>G, and g.28650A>G. In yet another embodiment, the single nucleotide polymorphism is g.−18C>A or g.1679C>G and the compound inhibits cholesterol absorption. In another embodiment, the haplotype is [A(−133), A(−18), G(1679)] or [G(−133), C(−18), C(1679)] and the compound is ezetimibe. The invention further relates to isolated nucleic acids including within their sequence at least one of NPC1L1 polymorphisms g.−133A>G, g.−18C>A, or g.28650A>G. The invention also includes nucleic acid primers and oligonucleotide probes capable of hybridizing to such nucleic acids and to diagnostic kits comprising one or more of such primers and probes for detecting such polymorphisms in the NPC1L1 gene. For example, one such embodiment includes an isolated polynucleotide consisting of at least 12 contiguous nucleotides of SEQ ID NO: 1 or the complement thereof, wherein the polynucleotide includes a single nucleotide polymorphism that has a adenine base at nucleotide position 5,285 of SEQ D NO: 1. In another embodiment the isolated polynucleotide includes a single nucleotide polymorphism that has an adenine base at nucleotide position 5,400 of SEQ ID NO: 1. In yet another embodiment the isolated polynucleotide includes a single nucleotide polymorphism that has a guanine base at nucleotide position 34,067 of SEQ ID NO: 1.

Another aspect of the invention provides a method of determining whether a subject has a genotype associated with a higher than average response of humans to an NPC1L1 antagonist. The method includes the step of determining whether the subject is heterozygous or homozygous for polymorphism g.−18C>A or g.1679C>G, or heterozygous or homozygous for haplotype [A(−133), A(−18), G(1679)], wherein the presence in the heterozygous or homozygous form of either one of or both of the polymorphisms, or the haplotype, indicates that the subject has a genotype associated with a higher than average response in humans to the NPC1L1 antagonist.

A subject can be identified as heterozygous or homozygous for a particular polymorphism or haplotype by determining whether the polymorphism or haplotype is present on at least one allele, or by determining the number of alleles containing the polymorphism or haplotype.

Another aspect of the present invention relates to a method of estimating the responsiveness of a subject to compounds, such as ezetimibe, that affect NPC1L1 function, i.e., inhibits intestinal cholesterol absorption. The method includes the steps of obtaining a biological sample from the subject; and determining the nucleotide base present at a position in SEQ ID NO: 1 in the biological sample, wherein the presence of a adenosine heterozygosity or homozygosity at position 5,400 of SEQ ID NO: 1 indicates that the subject is statistically more likely to have a higher than average response to the compound than an individual lacking the adenosine heterozygosity or homozygosity. In another embodiment of the invention, the presence of a guanine heterozygosity or homozygosity at position 7,096 of SEQ ID NO: 1 indicates that the subject is statistically more likely to have a higher than average responsive to the compound than an individual lacking the guanine heterozygosity or homozygosity. In another embodiment of the invention, the presence of haplotype [A(−133), A(−18), G(1679)] heterozygosity or homozygosity indicates that the subject is statistically more likely to have a higher than average responsive to the compound than an individual lacking the [A(−133), A(−18), G(1679)] haplotype.

Another aspect of the invention provides a method for detecting a predisposition to a health risk level of plasma cholesterol in a human subject. The method includes detecting in the human subject the presence or absence of a polymorphism in the genomic sequence of a human NPC1L1 allele, wherein the human NPC1L1 allele consists of a guanine at position 34,067 of SEQ ID NO: 1. The presence of the guanine is indicative of a predisposition to a health risk level of plasma cholesterol in the subject.

The inventive methods of the invention include any assay that allows determination of nucleotide base present in any of the above described polymorphisms and haplotypes. Exemplary assays include, but are not limited to, direct nucleotide sequence analysis, differential nucleic acid hybridization analysis, including DNA microarray analysis, restriction fragment length polymorphism analysis, and polymerase chain reaction analysis.

Another aspect of the invention provides a method of reducing cholesterol in a patient. The method comprises the step of administering to the patient an effective amount of an NPC1L1 antagonist, wherein the patient is identified as having a SNP selected from the group consisting of g.−18C>A and g.1679C>G. In another embodiment, the patient is identified as having an [A(−133), A(−18), G(1679)] haplotype

Another aspect of the invention provides a diagnostic kit comprising at least one allele-specific nucleic acid primer capable of detecting a polymorphism in the NPC1L1 gene at one or more of positions 5,285, 5,400, 7,096, and 34,067 of SEQ ID NO: 1 and an oligonucleotide probe for detecting a polymorphism in the NPC1L1 gene capable of hybridizing specifically to a nucleic acid wherein the nucleotide polymorphism in the NPC1L1 gene is selected from at least one of an A or a G at position 5,285 in SEQ ID NO: 1, a C or an A at position 5,400 in SEQ ID NO: 1, a C or a G at position 7,096 in SEQ ID NO: 1, and an A or a G at position 34,067 in SEQ ID NO. 1, and combinations thereof as well as their reverse complement.

### BRIEF DESCRIPTION OF THE DRAWINGS

FIG. 1A. D′ plot for common variants identified in the resequencing cohort. D′ plot was generated by the Haploview software program. The triangular matrix represents the D′ values computed between all pairs of common SNPs in the Caucasian ethnic group. White indicates low D′ values indicating no or weak linkage disequilibrium between SNPs, the narrowest slanted striped lines indicates high D′ values indicating significant linkage disequilibrium between SNPs, and speckled pattern indicates high D′ values with low log of odds ratios.

FIG. 1B. D′ plot for genotypes tested in the EASE cohort. D′ plot was generated by the Haploview software program. The triangular matrix represents the D′ values computed between all pairs of common SNPs in the Caucasian ethnic group. White indicates low D′ values indicating no or weak linkage disequilibrium between SNPs, the narrowest slanted striped lines indicates high D′ values indicating significant linkage disequilibrium between SNPs, and speckled pattern indicates high D′ values with low log of odds ratios.

FIG. 2. Common haplotypes identified in the EASE cohort. Each column represents one of the 12 common SNPs genotyped in the EASE cohort (see Example 1, Table 4). Each row represents a 7p13 chromosome, where a random set of 250 7p13 chromosomes was sampled from the 2,430 7p13 chromosomes observed in the EASE cohort. Minor alleles for each SNP are shaded with narrow slanted stripes, while the common alleles are shaded with wider slanted stripes. The six SNPs highlighted in bold text signify those tagging SNPs that uniquely identify the eight common haplotypes represented in this plot. These six SNPs were used in the association study described in Example 3 for ezetimibe response.

### DETAILED DESCRIPTION OF THE INVENTION

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims. Also, as used herein and in the appended claims, the singular forms “a”, “an”, and “the” include plural referents unless the context clearly dictates otherwise. Thus, for example, reference to “a complex” includes a plurality of such complexes and reference to “the formulation” includes reference to one or more formulations and equivalents thereof known to those skilled in the art, and so forth.

### I. Definitions

Unless defined otherwise, all technical and scientific terms used herein have the meaning commonly understood by one of ordinary skill in the art to which this invention belongs.

As used herein, “[A(−133), A(−18), G(1679)]” refers to an NPC1L1 haplotype composed of an adenine base at a nucleotide position corresponding to 5,285 of SEQ ID NO: 1, an adenine base at a nucleotide position corresponding to 5,400 of SEQ ID NO: 1 and a guanine base at a nucleotide position corresponding to 7,096 of SEQ ID NO: 1. Reference to “corresponding” indicates the position of each polymorphism in the haplotype with respect to SEQ ID NO: 1. In some contexts, it will be evident that the designation [A(−133), A(−18), G(1679)] refers to a subhaplotype that may be present on two or more haplotype alleles of the NPC1L1 gene.

As used herein, “[G(−133), C(−18), C(1679)]” refers to a haplotype composed of a guanine base at a nucleotide position corresponding to 5,285 of SEQ ID NO: 1, a cytosine base at a nucleotide position corresponding to 5,400 of SEQ ID NO: 1 and a cytosine base at a nucleotide position corresponding to 7,096 of SEQ ID NO: 1. Reference to “corresponding” indicates the position of each polymorphism in the haplotype with respect to SEQ ID NO: 1. In some contexts, it will be evident that the designation [G(−133), C(−18), C(1679)] refers to a subhaplotype that may be present on two or more haplotype alleles of the NPC1L1 gene.

As used herein, “g.−133A>G” refers to a guanine base at a nucleotide position corresponding to 5,285 of SEQ ID NO: 1, or position located 133 bases upstream of the ATG start codon of the NPC1L1 gene in genomic DNA. Reference to “corresponding” indicates the position of the polymorphism with respect to SEQ ID NO: 1. The g.−133A>G polymorphism may be present in other sequences related to SEQ ID NO: 1, e.g., the sequence may contain other NPC1L1 gene polymorphisms.

As used herein, “g.−18C>A” refers to an adenine base at a nucleotide position corresponding to 5,400 of SEQ ID NO: 1, or position located 18 bases upstream of the ATG start codon of the NPC1L1 gene in genomic DNA. Reference to “corresponding” indicates the position of the polymorphism with respect to SEQ ID NO: 1. The g.−18C>A polymorphism may be present in other sequences related to SEQ ID NO: 1, e.g., the sequence may contain other NPC1L1 gene polymorphisms.

As used herein, “g.1679C>G” refers to an guanine base at a nucleotide position corresponding to 7,096 of SEQ ID NO: 1, or position located 1679 bases downstream of the ATG start codon of the NPC1L1 gene in genomic DNA. Reference to “corresponding” indicates the position of the polymorphism with respect to SEQ ID NO: 1. The g. 1679C>G polymorphism may be present in other sequences related to SEQ ID NO: 1, e.g., the sequence may contain other NPC1L1 gene polymorphisms.

As used herein, “g.28650A>G” refers to a guanine base at a nucleotide position corresponding to 34,067 of SEQ ID NO: 1. Reference to “corresponding” indicates the position of the polymorphism with respect to SEQ ID NO: 1, or located 28,650 bases downstream of the ATG start codon of the NPC1L1 gene in genomic DNA. The g.28650A>G polymorphism may be present in other sequences related to SEQ ID NO: 1, e.g., the sequence may contain other NPC1L1 gene polymorphisms.

As used herein, “allele” is a particular nucleotide sequence of a gene or other genetic locus. An allele may comprise one or more SNPs, or one of the haplotypes described herein for a specified combination of polymorphic sites in the NPC1L1 gene. Reference to allele may includes the form of a locus that is present on a single chromosome 7 in a somatic cell obtained from an individual; since chromosome 7 an autosomal chromosome, then the somatic cell in the individual will normally have two alleles for the locus. An individual with two alleles that are the same is homozygous for that locus. An individual with two different alleles for a locus is heterozygous.

As used herein, “NPC1L1 antagonist”,includes any compound, substance or agent including, without limitation, a small molecule, protein, antibody or nucleic acid, that inhibits, directly or indirectly, to any degree, the uptake of dietary cholesterol and/or related phytosterols by NPC1LL. Preferably an NPC1L1 antagonist binds to NPC1L1, and preferably significantly inhibits NPC1L1 activity. Reference to “NPC1L1 antagonist” does not indicate a particular mode of action. Ezetimibe is an example of an NPC1L1 antagonist.

As used herein, “genotype” is an unphased 5′ to 3′ sequence of the two alleles, typically a nucleotide pair, found at each polymorphic site in a set of one or more polymorphic sites in a locus on a pair of homologous chromosomes in an individual.

As used herein, “genotyping” is a process for determining a genotype of an individual.

As used herein, “haplotype pair” refers to the two haplotypes found for a locus in a single individual.

As used herein, “haplotyping” refers to any process for determining one or more haplotypes in an individual, including the haplotype pair for a particular set of PSs, and includes use of family pedigrees, molecular techniques and/or statistical inference.

As used herein, “increased ezetimide response” refers to an increased mean percentage decrease in LDL-C due to ezetimide treatment in a group of patients defined by a genotype compared to patients having a different genotype. Ezetimide treatment includes administering ezetimibe or NPC1L1 antagonist, as monotherapy or in combination with at least one other compound used to lower LDL-C. The increased mean percentage deceases is statistically significant in the different groups defined by their genotype. In some embodiments, the individual and the population are of similar ethnic or geographic origin. In some embodiments, the therapeutic regimen comprises at least six weeks of treatment with 10 mg/day ezetimibe and the mean decrease in LDL-C in the group having the NPC1L1 marker is at least 15% greater than the mean LDL-C decrease in the group lacking the NPC1L1 marker. In a preferred embodiment, the increased ezetimibe response is at least a mean decrease in LDL-C of at least 27%. In another particularly preferred embodiment, the NPC1L1 plus and minus groups are comprised only of those individuals who are extreme responders to ezetimibe, i.e., whose percentage LDL-C decrease falls within the upper or lower 10^{th} percentile of the response distribution observed in a clinical study of ezetimibe. A preferred increased ezetimibe response in extreme responders with a NPC1L1 marker is a −34% change in LDL-C as compared to a −17% change in LDL-C in extreme responders lacking the marker.

As used herein, “increased LDL-C response to an NPC1L1 antagonist” refers to an increased mean percentage decrease in LDL-C due to NPC1L1 antagonist treatment in a group of patients defined by a genotype compared to patients having a different genotype. NPC1L1 antagonist treatment, includes administering NPC1L1 antagonist, as monotherapy or in combination with at least one other compound used to lower LDL-C. The increased mean percentage deceases is statistically significant in the different groups defined by their genotype. In some embodiments, the individual and the population are of similar ethnic or geographic origin. In some embodiments, the therapeutic regimen comprises at least six weeks of treatment with a therapeutically effective amount of NPC1L1 antagonist and the mean decrease in LDL-C in the group having the NPC1L1 marker is at least 15% greater than the mean LDL-C decrease in the group lacking the NPC1L1 marker. In a preferred embodiment, the increased LDL-C response to the NPC1L1 antagonist is at least a mean decrease in LDL-C of at least 20%. In another particularly preferred embodiment, the NPC1L1 plus and minus groups are comprised only of those individuals who are extreme responders to the NPC1L1 antagonist, i.e., whose percentage LDL-C decrease falls within the upper or lower 10^{th} percentile of the response distribution observed in a clinical study of the NPC1L1 antagonist.

As used herein, an “isolated polynucleotide” is a nucleic acid molecule that exists in a physical form that is nonidentical to any nucleic acid molecule of identical sequence as found in nature.

As used herein, “locus” refers to a location on a chromosome or DNA molecule. A locus may correspond to a gene or portion thereof, other genomic region(s) associated with a phenotype, and single polymorphic site or a specific combination of polymorphic sites in a specified genomic region.

As used herein, “normal” as used herein in connection with the quantity, in a subject, of a clinical parameter (such as LDL-C) means a specific number or numerical range of that parameter that is typically observed in healthy subjects of similar age, weight, and/or gender, or that a clinician who practices in the relevant field would understand as being normal. Conversely, “abnormal” refers to a specific number or numerical range for a clinical parameter that is lower or higher than a normal number or normal numerical range, or that a clinician practicing in the field would understand to be abnormal.

As used herein, “NPC1L1” refers to human Niemann Pick C1-Like 1 protein (AAR97886).

As used herein, “NPC1L1” refers to polynucleotides encoding NPC1L1.

As used herein, the “NPC1L1 gene” refers to the sequence present within the nucleic acid sequences in SEQ ID NO: 1 located on human chromosome 7p13. The NPC1L1 gene includes 20 exon regions, 19 intron sequences intervening the exon sequences and 3′ and 5′ untranslated regions (3UTR and 5′UTR) including the promoter region of the NPC1L1 gene sequence set forth in SEQ ID NO: 1. The first in frame ATG occurs in exon 1 (or at position 5,418 in SEQ ID NO: 1) while the TGA stop codon occurs in exon 20 (or at position 33,228 in SEQ ID NO: 1).

As used herein, “NPC1L1 marker” in the context of the present invention is a specific copy number of a specific genetic variant that is associated with a health risk level of LDL-C or an increased ezetimibe response. Preferred NPC1L1 markers are those shown in Table 1, as well as genetic markers in which at least one variant in any marker in Table 1 is replaced by the same copy number of a substitute haplotype or a linked variant, each of which is referred to herein as an alternate genetic marker. A substitute haplotype comprises a sequence that is similar to that of any of the haplotypes shown in Table 1, but in which the allele at one but less than all of the specifically identified polymorphic sites in that haplotype has been substituted with the allele at a different polymorphic site, which substituting allele is in high linkage disequilibrium (LD) with the allele at the specifically identified polymorphic site. A linked variant is any type of variant, including a SNP or haplotype, which is in high LD with any one of the variants shown in Table 1. Two particular alleles at different loci on the same chromosome are said to be in LD if the presence of one of the alleles at one locus tends to predict the presence of the other allele at the other locus. Alternate genetic markers, which are further described below, may comprise types of variations other than SNPs, such as indels, RFLPs, repeats, etc.

As used herein, “nucleotide pair” is the set of two nucleotides (which may be the same or different) found at a polymorphic site on the two copies of a chromosome from an individual.

As used herein, “pharmacogenetic indication” refers to a genetic profile that identifies individuals whom a drug is intended to treat, in addition to the disease for which drug is indicated. The genetic profile comprises the presence of an NPC1L1 drug response marker. In preferred embodiments, the genetic-profile comprises the presence of an NPC1L1 marker that is associated with a health-risk level of LDL-C.

As used herein, “phased sequence” refers to the combination of nucleotides present on a single chromosome at a set of polymorphic sites, in contrast to an unphased sequence, which is typically used to refer to the sequence of nucleotide pairs found at the same set of PS in both chromosomes.

As used herein, “polymorphic site” or “PS” refers to the position in a genetic locus or gene at which a SNP or other nonhaplotype polymorphism occurs. A PS is usually preceded by and followed by highly conserved sequences in the population of interest and thus the location of a PS is typically made in reference to a consensus nucleic acid sequence of thirty to sixty nucleotides that bracket the PS, which in the case of a SNP polymorphism is commonly referred to as the “SNP context sequence”. The location of the PS may also be identified by its location in a consensus or reference sequence relative to the initiation codon (ATG) for protein translation. The skilled artisan understands that the location of a particular PS may not occur at precisely the same position in a reference or context sequence in each individual in a population of interest due to the presence of one or more insertions or deletions in that individual as compared to the consensus or reference sequence. Moreover, it is routine for the skilled artisan to design robust, specific and accurate assays for detecting the alternative alleles at a polymorphic site in any given individual, when the skilled artisan is provided with the identity of the alternative alleles at the PS to be detected and one or both of a reference sequence or context sequence in which the PS occurs. Thus, the skilled artisan will understand that specifying the location of any PS described herein by reference to a particular position in a reference or context sequence (or with respect to an initiation codon in such a sequence) is merely for convenience and that any specifically enumerated nucleotide position literally includes whatever nucleotide position the same PS is actually located at in the same locus in any individual being tested for the presence or absence of a genetic marker of the invention using any of the genotyping methods described herein or other genotyping methods well-known in the art.

As used herein, “polymorphism” refers to the occurrence of two or more genetically determined alternative sequences or alleles that occur for a gene or a locus in a population. A human individual may be homozygous or heterozygous for the different alleles that exist. The different alleles of a polymorphism typically occur in a population at different frequencies with the allele occurring most frequently in a selected population sometimes references as the “major” or “wildtype” allele. A biallelic polymorphism has two alleles, and the minor allele may occur at any frequency greater than zero and less than 50% in a selected population, including frequencies of between 1% and 2%, 2% and 10%, 10% and 20%, 20% and 30%, etc. SNPs are typically bi-allelic polymorphisms. A triallelic polymorphism has three alleles. Preferably, the term polymorphism is used to describe a polymorphic locus at which each allele occurs at a frequency of greater than 1%, and more preferably 5%. Types of polymorphisms include sequence variation at a single polymorphic site, such as single nucleotide polymorphisms or SNPs, and variation in the sequence of nucleotides that occur on a single chromosome at a set of two or more polymorphic sites in the gene or locus of interest. Each sequence that occurs for a specific set of polymorphic sites is an allele for that locus and is also referred to herein as a haplotype. In addition, to SNPs and haplotypes, examples of polymorphisms include restriction fragment length polymorphisms (RFLPs), variable number of tandem repeats (VNTRs), dinucleotide repeats, trinucleotide repeats, tetranucleotide repeats, simple sequence repeats, insertion elements such as Alu, and deletions of one or more nucleotides.

As used herein, “purified nucleic acid” represents at least 10% of the total nucleic acid present in a sample or preparation. In preferred embodiments, the purified nucleic acid represents at least about 50%, at least about 75%, or at least about 95% of the total nucleic acid in an isolated nucleic acid sample or preparation. Reference to “purified nucleic acid” does not require that the nucleic acid has undergone any purification and may include, for example, chemically synthesized nucleic acid that has not been purified.

As used herein, “polynucleotide” and “nucleic acid” refer to single or double-stranded molecules which may be DNA, comprised of the nucleotide bases A (adenine), T (thymine), C (cytosine) and G (guanine), or RNA, comprised of the bases A, U (uracil) (substitutes for T), C, and G. The polynucleotide may represent a coding strand or its complement. Polynucleotide molecules or nucleic acids encoding for proteins may be identical in sequence to the sequence which is naturally occurring or may include alternative codons which encode the same amino acid as that which is found in the naturally occurring sequence (See, Lewin “Genes V” Oxford University Press Chapter 7, 1994, 171-174. Furthermore, such encoding molecules may include codons which represent conservative substitutions of amino acids as described. For example, polynucleotide may represent genomic DNA, mRNA, cDNA, primers and probes.

As used herein, “treat” or “treating” means administering an effective amount of a drug internally or externally to a patient to alleviate one or more disease symptoms in the treated patient, whether by inducing the regression of or inhibiting the progression of such symptom(s) by any clinically measurable degree. The amount of a drug that is effective to alleviate any particular disease symptom (also referred to as the “therapeutically effective amount”) may vary according to factors such as the disease state, age, and weight of the patient, and the ability of the drug to elicit a desired response in the patient. Whether a disease symptom has been alleviated can be assessed by any clinical measurement typically used by physicians or other skilled healthcare providers to assess the severity or progression status of that symptom. While an embodiment of the present invention (e.g., a treatment method or article of manufacture) may not be effective in alleviating the target disease symptom(s) in every patient, it should alleviate the target disease symptom(s) in a statistically significant number of patients as determined by any statistical test known in the art such as the Student's t-test, the chi²-test, the U-test according to Mann and Whitney, the Kruskal-Wallis test (H-test), Jonckheere-Terpstra-test and the Wilcoxon-test.

### II. Composition and Phenotypic Effect of NPC1L1 Markers of the Invention

As described above and in the examples below, NPC1L1 markers according to the present invention predict a particular phenotype, i.e., either a health risk level of LDL-C or an increased average response to ezetimibe, which is likely to be exhibited by an individual in whom the NPC1L1 marker is present. Each NPC1L1 marker of the invention is a combination of a particular allele associated with one of these phenotypes and a copy number of that allele.

Table 1 lists preferred NPC1L1 markers of the invention. An individual having NPC1L1 marker 1 (e.g., at least one copy of 34067G) is more likely to have a health risk level of LDL-C than an individual lacking NPC1L1 marker 1 (e.g., zero copies of 34067G). An individual having at least one copy NPC1L1 marker 2, 3, 4 or 5 is likely to exhibit an increased ezetimibe response, relative to the ezetimibe response of individuals lacking NPC1L1 marker 2, 3, 4 or 5, respectively.

TABLE 1
NPC1L1 Markers

| | | Copy No. | |
|---|---|---|---|
| Marker | Variant^{a} | of Variant | Phenotype^{b} |
| 1 | 34067G | 1 or 2 | Health Risk Level of |
| | (28650G) | | LDL-C |
| 2 | 5400A | 1 or 2 | Increased Ezetimibe |
| | (−18A) | | Response |
| 3 | 7096G | 1 or 2 | Increased Ezetimibe |
| | (1679G) | | Response |
| 4 | 5285A, 5400A, 7096G | 1 or 2 | Increased Ezetimibe |
| | (−133A, −18A, 1679G) | | Response |
| 5 | 5285G, 5400C, 7096C | 0 | Increased Ezetimibe |
| | (−133G, −18C, 1679C) | | Response |

^{a}The numbers designate the location of a polymorphic site in the NPC1L1 gene, either by reference to its distance from the first nucleotide position in SEQ ID NO: 1 (first line) or its distance from the ATG start codon in SEQ ID NO: 1 (parenthesis); the letter refers to the nucleotide allele present at that site.
^{b}As defined in the Detailed Description.

The polymorphic sites comprising these NPC1L1 markers are located in the NPC1L1 locus at positions corresponding to those identified in the above Definitions and SEQ ID NO: 1. In describing the polymorphic sites in the markers of the invention, reference is made to the sense strand of the gene for convenience. However, as recognized by the skilled artisan, nucleic acid molecules containing the NPC1L1 gene may be complementary double stranded molecules and thus reference to a particular site on the sense strand also refers to the corresponding site on the complementary antisense strand.

In addition, the skilled artisan will appreciate that all of the embodiments of the invention described herein may be practiced using an alternate genetic marker for any of the genetic markers in Table 1. Alternate genetic markers comprising a substitute haplotype are readily identified by determining the degree of linkage disequilibrium (LD) between an allele at a PS in one of the markers in Table 1 and a candidate substituting allele at a polymorphic site located elsewhere in the NPC1L1 gene or on chromosome 7. Similarly, alternate genetic markers comprising a linked variant are readily identified by determining the degree of LD between a haplotype in Table 1 and a candidate linked variant located elsewhere in the NPC1L1. The candidate substituting allele or linked variant may be an allele of a polymorphism that is currently known. Other candidate substituting alleles and linked variants may be readily identified by the skilled artisan using any technique well-known in the art for discovering polymorphisms.

The degree of LD between a genetic marker in Table 1 and a candidate alternate marker may be determined using any LD measurement known in the art. LD patterns in genomic regions are readily determined empirically in appropriately chosen samples using various techniques known in the art for determining whether any two alleles (e.g., between SNPs at different PSs or between two haplotypes) are in linkage disequilibrium (see, e.g., GENETIC DATA ANALYSIS II, Weir, Sineuer Associates, Inc. Publishers, Sunderland, Mass. 1996). The skilled artisan may readily select which method of determining LD will be best suited for a particular sample size and genomic region.

One of the most frequently used measures of linkage disequilibrium is Δ² which is calculated using the formula described by Devlin et al. (Genomics, 29(2):311-22 (1995)). Δ² is the measure of how well an allele X at a first locus predicts the occurrence of an allele Y at a second locus on the same chromosome. The measure only reaches 1.0 when the prediction is perfect (e.g. X if and only if Y).

In preferred alternate genetic markers, the locus of a substituting allele or a linked variant is in a genomic region of about 100 kilobases spanning the NPC1L1 gene, and more preferably, the locus is in the NPC1L1 gene. Other preferred alternate genetic markers are those in which the LD between the relevant alleles (e.g., between the substituting SNP and the substituted SNP, or between the linked variant and the haplotype in the marker) has a Δ² value, as measured in a suitable reference population, of at least 0.75, more preferably at least 0.80, even more preferably at least 0.85 or at least 0.90, yet more preferably at least 0.95, and most preferably 1.0. The reference population used for this Δ² measurement preferably reflects the genetic diversity of the population of patients to be treated with a drug containing a NPC1L1 antagonist. For example, the reference population may be the general population, a population using the drug, a population diagnosed with a particular condition for which the drug shows efficacy (such as hypercholesterolemia) or a population of similar ethnic background.

In all of the embodiments of the invention described herein, the skilled artisan will appreciate that detecting the presence or absence in an individual of a particular NPC1L1 marker in Table 1 is literally equivalent to detecting the presence or absence of an alternate genetic marker when there is perfect linkage disequilibrium between the alleles in the Table 1 marker and the alternate marker.

In one aspect, the invention provides a means to classify a patient in need of cholesterol therapy into response groups based upon objective genetic criteria. In addition, based upon which class a patient is within, the invention provides an objective basis for selecting the most appropriate drug therapy for that patient. In another aspect the invention provides a method for identification of additional NPC1L1 polymorphisms that can be used to screen and develop therapeutic agents that can be used to treat or prevent health risk levels of cholesterol and/or a health risk cholesterol-associated condition.

Various aspects of the invention are based on the discovery of single nucleotide polymorphisms (SNP) in the NPC1L1 gene. In particular, a novel g.−18C>A polymorphism in the NPC1L1 gene (at position 5,400 of SEQ ID NO: 1) was identified in the promoter region of the NPC1L1 gene. Statistical analysis of genotyping results and blood component measurement results showed that the presence of the g.−18C>A polymorphism, in either the homozygous or heterozygous state, i.e., one copy or two copies, is significantly associated with changes in total cholesterol, LDL-C, non-HDL-C and apoB levels in response to treatment with ezetimibe as compared to individuals homozygous for the major allele, i.e., having a cytosine at position 5,400 of SEQ ID NO: 1. Another NPC1L1 polymorphism, g1679C>G (alternative NCBI designation, rs2072183) was also found to be associated with changes in LDL-C levels in response to treatment with ezetimibe as compared to individuals homozygous for the major allele, i.e., having a cytosine at position 7,096 of SEQ ID NO: 1. Haplotype analysis also identified two NPC1L1 haplotypes, comprising three SNPs, that are significantly associated with changes in LDL-C levels in response to treatment with ezetimibe. Haplotype [A(−133), A(−18), G(1679)] was found to be associated with a higher than average response to ezetimibe treatment, i.e., lowering of LDL-C, compared to individuals having a different haplotype at positions 5,285, 5,400 and 7,096 of SEQ ID NO: 1. Haplotype [G(−133), C(−18), C(1679)] was found to be associated with a lower than average response to ezetimibe treatment, i.e., lowering of LDL-C, compared to individuals having a different haplotype at positions 5,285, 5,400 and 7,096 of SEQ ID NO: 1. The genetic association between these NPC1L1 variants and LDL-C response to ezetimibe treatment supports NPC1L1's role as a key gene for cholesterol absorption in pathways that are sensitive to ezetimibe treatment.

Another aspect of the invention relates to a method for correlating a single nucleotide polymorphism or haplotype in the NPC1L1 gene with the efficacy of a pharmaceutically active compound administered to a subject which method comprises determining a single nucleotide polymorphisms or a haplotype in the NPC1L1 gene of a subject and determining the status of the subject to which a pharmaceutically active compound was administered by reference to the polymorphism or haplotype in the NPC1L1 gene. In one embodiment, the status of the subject is based upon measurement a disease state before and after administration of the compound. The efficacy of the pharmaceutically active compound administered to the subject is evaluated by determining whether a particular single nucleotide polymorphism or a particular haplotype is correlated with a statistically significant change in the status of the subject in response to administration of the compound as compared to the change in status of individuals having a different genotype at the polymorphic sequence position or haplotype sequence positions. Exemplary disease states include atherosclerosis, acute coronary syndrome, coronary artery disease and the like. Usually, but not always, the disease state is associated with blood or blood plasma cholesterol levels or blood protein associated lipids levels, such as, for example, low density lipid cholesterol, total cholesterol, non-high density lipid cholesterol and apolipoprotein B (apoB).

According to a further aspect of the present invention there is provided a method for correlating single nucleotide polymorphisms in the NPC1L1 gene with the efficacy of a pharmaceutically active compound administered to a human subject which method comprises determining single nucleotide polymorphisms in the NPC1L1 gene of a human subject and determining the status of said human being to which a pharmaceutically active compound was administered by reference to polymorphism at least one or more positions of SEQ ID NO: 1 comprising the NPC1L1 gene including positions 5,285, 5,400, 7,096, and, or 34,067. The status of the human subject may be determined by reference to allelic variation at one, two, three, four, or all four positions. The status of the human subject may also be determined by one or more of the specific polymorphisms identified herein in combination with one or more other single nucleotide polymorphisms.

Another aspect of the invention provides a method of predicting responsiveness of a subject to a drug affecting NPC1L1 function. The method includes obtaining a biological sample from a subject; and determining the nucleotide base present at a position of SEQ ID NO: 1 in the biological sample wherein the position is selected from the group consisting of position 5,400 and position 7,096; wherein the presence of an adenine base at position 5,400 or a guanine at position 7,096 is indicative of an increased level of responsiveness of the subject to the drug. In another embodiment, the presence of a cytosine base at position 5,400 or a cytosine base at position 7,096 of SEQ ID NO: 1 is indicative of a decreased level of responsiveness of the subject to the drug.

Another aspect of the invention provides a method for detecting a predisposition to a health risk level of plasma low density lipid cholesterol in a human subject. The method includes detecting in the subject the presence of a polymorphism in the genomic sequence of a human NPC1L1 allele, wherein the human NPC1L1 allele consists of a guanine at position 34,067 of SEQ ID NO: 1. The presence of the guanine base at position 34,067 is indicative of the predisposition of the subject to a health risk level of plasma cholesterol. In another embodiment, the detection of the guanine base at position 34,067 is indicative of the predisposition of the subject to coronary heart disease (CHD).

In one embodiment of the invention, a health risk level of LDL-C is determined by reference to guidelines set forth by an educational, medical, governmental, or other agency accepted by persons of skill in the art. For example, in the United States the National Cholesterol Education Program periodically issues reports detailing the health risks associated with various cholesterol levels. In particular, the NCEP Adult Treatment Panel issued guidelines that establish specific LDL-C target levels according to the level of CHD risk (JAMA (2001) 285:2486-97). Recently, based on emerging clinical trial data, an update to these guidelines has established an optional target of LDL-C<70 mg/dL for persons considered to be at very high risk (Circulation (2004) 110:227-239). In the practice of the present invention, a level of plasma low density lipid cholesterol that puts a person at risk is determined based upon the updated NCEP ATP guidelines (Circulation (2004) 110:227-239). In one embodiment, a health risk level of plasma low density lipid cholesterol is between about 70 mg/dL and about 130 mg/dL.

According to another aspect of the invention a method is provided for determining whether a patient has a genotype associated with an above average increase in response to an NPC1L1 antagonist comprising the step of determining whether the patient has a genotype selected from the group consisting of an adenine base heterozygosity or homozygosity at position 5,400 of SEQ ID NO: 1, a guanine base heterozygosity or homozygosity at position 7,096 of SEQ ID NO: 1, and a [A(−133), A(−18), G(1679)]haplotype heterozygosity or homozygosity corresponding to positions 5,285, 5400 and 7,096 of SEQ ID NO: 1. In some embodiments the patient has a health risk level of cholesterol. In other embodiments, the patient is currently or has previously undergone statin treatment. Exemplary statins are described below in more detail. In other embodiments, the patient has failed to achieve a sufficient reduction in cholesterol using a statin treatment. A sufficient reduction in cholesterol for a patient may be determined by reference to any art accepted cholesterol target level given various characteristics of the patient, e.g., age, general health, etc. In particular, such target levels and health risk factors are described in a variety of materials prepared by educational, medical or governmental agencies. In a particular embodiment, the cholesterol target level for a patient is determined by reference to NCEP ATP guidelines. In one embodiment, a sufficient reduction in plasma LDL-C is achieved when the patient has a plasma level of LDL-C of less than about 100 mg/dL, or less than about 70 mg/dL.

Another aspect of the invention provides a method of reducing cholesterol in a patient comprising the step of administering to the patient an effective amount of an NPC1L1 antagonist, wherein the patient is identified as having a genotype selected from the group consisting of an adenine base heterozygosity or homozygosity at position 5,400 of SEQ ID NO: 1, a guanine base heterozygosity or homozygosity at position 7,096 of SEQ ID NO: 1, and a [A(−133), A(−18), G(1679)] haplotype heterozygosity or homozygosity corresponding to positions 5,285, 5400 and 7,096 of SEQ ID NO: 1. A patient is identified as having one of the above identified genotypes by obtaining a biological sample from the patient and determining which nucleotide base is present at the corresponding position of the NPC1L1 gene sequence. A patient genotype is identified when it is known that the patient has one of the genotypes identified herein, e.g., one of the NPC1L1 markers described above. An effective amount of an NPC1L1 antagonist is an amount that reduces intestinal transport of cholesterol. For example, in one embodiment, the NPC1L1 antagonist is ezetimibe and the effective amount is 10 milligrams, administered once daily. Other NPC1L1 antagonists are described herein below.

Another aspect of the invention includes a method for advertising a drug product comprising ezetimibe comprising promoting, to a target audience, the use of the drug product for treating high cholesterol or a high cholesterol-related disease in patients possessing a single nucleotide polymorphism selected from the group consisting of g.−133A>G, g.−18C>A and g.28650A>G or haplotype [A(−133), A(−18), G(1679)], wherein an individual possessing the selected single nucleotide polymorphism or haplotype is more likely to exhibit a higher than average responsive to ezetimibe than an individual lacking the selected single nucleotide polymorphism or haplotype.

In the context of the present invention, manipulation of nucleic acid molecules derived from the tissues of human subjects can be effected to provide for the analysis of NPC1L1 genotypes, and for screening and diagnostic methods relating to the NPC1L1 SNP and haplotype markers, in particular, one or more SNPs selected from NPC1L1-g.−133A>G, NPC1L1-g.−18C>A, NPC1L1−g.1679C>G, and NPC1L1-g.28650A>G, or one or more three-SNP haplotypes selected from [A(5285)-A(5400)-G(7096) and [G(5285)-C(5400)-C(7096)]. Nucleic acid molecules utilized in these contexts can be amplified, as described below, and generally include RNA, genomic DNA, and cDNA derived from RNA.

### III. Polynucleotides and Polynucleotide Screening Methods

The presence in an individual of an NPC1L1 marker may be determined by any of a variety of methods well known in the art that permits the determination of whether the individual has the required copy number of the variant comprising the marker. For example, if the required copy number is 1 or 2, then the method need only determine that the individual has at least one copy of the variant. In preferred embodiments, the method provides a determination of the actual copy number.

Typically, these methods involve assaying a nucleic acid sample prepared from a biological sample obtained from the individual to determine the identity of a nucleotide or nucleotide pair present at one or more polymorphic sites in the marker. Nucleic acid samples may be prepared from virtually any biological sample. For example, convenient samples include whole blood serum, semen, saliva, tears, fecal matter, urine, sweat, buccal matter, skin and hair. Somatic cells are preferred if determining the actual copy number of the marker variant. Nucleic acid samples may be prepared for analysis using any technique known to those skilled in the art. Preferably, such techniques result in the production of genomic DNA sufficiently pure for determining the genotype or haplotype pair for a desired set of polymorphic sites in the nucleic acid molecule. Such techniques may be found, for example, in Sambrook, et al., Molecular Cloning: A Laboratory Manual (Cold Spring Harbor Laboratory, New York) (2001).

For markers in which the specified polymorphism is a haplotype, the copy number of the haplotype in the nucleic acid sample may be determined by a direct haplotyping method or by an indirect haplotyping method, in which the haplotype pair for the set of polymorphic sites comprising the marker is inferred from the individual's haplotype genotype for that set of PSs. The way the nucleic acid sample is prepared depends on whether a direct or indirect haplotyping method is used.

Direct haplotyping, or molecular haplotyping, methods typically involve treating a genomic DNA sample isolated from a blood or cheek sample obtained from the individual in a manner that produces a hemizygous DNA sample that contains only one of the individual's two alleles for the locus which, as readily understood by the skilled artisan, may be the same allele or different alleles, and detecting the nucleotide present at each PS of interest. The nucleic acid sample may be obtained using a variety of methods known in the art for preparing hemizygous DNA samples, which include: targeted in vivo cloning (TIVC) in yeast as described in WO 98/01573, U.S. Pat. No. 5,866,404, and U.S. Pat. No. 5,972,614; generating hemizygous DNA targets using an allele specific oligonucleotide in combination with primer extension and exonuclease degradation as described in U.S. Pat. No. 5,972,614; single molecule dilution (SMD) as described in Ruaño et al., Proc. Natl. Acad. Sci. 87:6296-300 (1990); and allele specific PCR (Ruaño et al., Nucl. Acids Res. 17:8392 (1989); Ruaño et al., Nucl. Acids Res. 19:6877-82 (1991); Michalatos-Beloin et al., supra).

As will be readily appreciated by those skilled in the art, any individual clone of the locus in an individual will permit directly determining the haplotype for only one of the two alleles; thus, additional clones will need to be examined to directly determine the identity of the haplotype for the other allele. Typically, at least five clones of the genomic locus present in the individual should be examined to have more than a 90% probability of determining both alleles. In some cases, however, once the haplotype for one allele is directly determined, the haplotype for the other allele may be inferred if the individual has a known genotype for the PSs comprising the marker or if the frequency of haplotypes or haplotype pairs for the locus in an appropriate reference population is available.

Direct haplotyping of both alleles may be performed by assaying two hemizygous DNA samples, one for each allele, that are placed in separate containers. Alternatively, the two hemizygous samples may be assayed in the same container if the two samples are labeled with different tags, or if the assay results for each sample are otherwise separately distinguishable or identifiable. For example, if the samples are labeled with first and second fluorescent dyes, and a PS in the locus is assayed using an oligonucleotide probe that is specific for one of the alleles and labeled with a third fluorescent dye, then detecting a combination of the first and third dyes would identify the nucleotide present at the PS in the first sample while detecting a combination of the second and third dyes would identify the nucleotide present at the PS in the second sample.

Indirect haplotyping methods typically involve preparing a genomic DNA sample isolated from a blood or cheek sample obtained from the individual in a manner that permits accurately determining the individual's genotype for each PS in the locus. The genotype is then used to infer the identity of at least one of the individual's haplotypes for the locus, and preferably used to infer the identity of the individual's haplotype pair for the locus.

In one indirect haplotyping method, the presence of zero, one or two copies of a haplotype of interest can be determined by comparing the individual's genotype for the PS in the marker with a set of reference haplotype pairs for the same set of PS and assigning to the individual a reference haplotype pair that is most likely to exist in the individual. The individual's copy number for the haplotype comprising the marker is the number of copies of that haplotype that are in the assigned reference haplotype pair.

The reference haplotype pairs are those that are known to exist in the general population or in a reference population. The reference population may be composed of randomly selected individuals representing the major ethnogeographic groups of the world. A preferred reference population is one having a similar ethnogeographic background as the individual being tested for the presence of the marker. The size of the reference population is chosen based on how rare a haplotype is that one wants to be guaranteed to see. For example, if one wants to have a q % chance of not missing a haplotype that exists in the population at a p % frequency of occurring in the reference population, the number of individuals (n) who must be sampled is given by 2n=log(1−q)/log(1−p) where p and q are expressed as fractions. A particularly preferred reference population includes one or more 3-generation families to serve as a control for checking quality of haplotyping procedures. If the reference population comprises more than one ethnogeographic group, the frequency data for each group is examined to determine whether it is consistent with Hardy-Weinberg equilibrium. Hardy-Weinberg equilibrium (D. L. Hartl et al., Principles of Population Genomics, Sinauer Associates (Sunderland, Mass.), 3^{rd} Ed., 1997) postulates that the frequency of finding the haplotype pair H₁/H₂ is equal to P_{H-W}(H₁/H₂)=2 p(H₁) p(H₂) if H₁≠H₂ and P_{H-W}(H₁/H₂)=p(H₁) p(H₂) if H₁═H₂. A statistically significant difference between the observed and expected haplotype frequencies could be due to one or more factors including significant inbreeding in the population group, strong selective pressure on the gene, sampling bias, and/or errors in the genotyping process. If large deviations from Hardy-Weinberg equilibrium are observed in an ethnogeographic group, the number of individuals in that group can be increased to see if the deviation is due to a sampling bias. If a larger sample size does not reduce the difference between observed and expected haplotype pair frequencies, then one may wish to consider haplotyping the individual using a direct, molecular haplotyping method.

Assignment of the haplotype pair may be performed by choosing a reference haplotype pair that is consistent with the individual's genotype. When the genotype of the individual is consistent with more than one reference haplotype pair, the frequencies of the reference haplotype pairs may be used to determine which of these consistent haplotype pairs is most likely to be present in the individual. If a particular consistent haplotype pair is more frequent in the reference population than other consistent haplotype pairs, then the consistent haplotype pair with the highest frequency is the most likely to be present in the individual. Occasionally, only one haplotype represented in the reference haplotype pairs is consistent with any of the possible haplotype pairs that could explain the individual's genotype, and in such cases the individual is assigned a haplotype pair containing this known haplotype and a new haplotype derived by subtracting the known haplotype from the possible haplotype pair. In rare cases, either no haplotypes in the reference population are consistent with the individual's genotype, or alternatively, multiple reference haplotype pairs are consistent with the genotype. In such cases, the individual is preferably haplotyped using a direct, molecular haplotyping method.

Any of all of the steps in the indirect haplotyping method described above may be performed manually, by visual inspection and performing appropriate calculations, but are preferably performed by a computer-implemented algorithm that accesses data on the individual's genotype and reference haplotype pairs stored in computer readable format. Such algorithms are described in WO 01/80156 and WO 2005048012A2. Alternatively, the haplotype pair in an individual may be predicted from the individual's genotype for that gene with the assistance of other reported haplotyping algorithms (e.g., Clark et al. 1990, Mol Bio Evol 7:111-22; PHASEv2 software (available for licensing from University of Washington Technology Transfer, and described in Stephens, M. et al., (2001) Am J Hum Genet 68:978-989); WO 02/064617; Niu T. et al (2002) Am J Hum Genet 70:157-169; Zhang et al. (2003) BMC Bioinformatics 4(1):3) or through a commercial haplotyping service such as offered by Genaissance Pharmaceuticals, Inc. (New Haven, Conn.).

All direct and indirect haplotyping methods described herein typically involve determining the identity of at least one of the alleles at a PS in a nucleic acid sample obtained from the individual. To enhance the sensitivity and specificity of that determination, it is frequently desirable to amplify from the nucleic acid sample one or more target regions in the locus. An amplified target region may span the locus of interest, such as an entire gene, or a region thereof containing one or more polymorphic sites. Separate target regions may be amplified for each PS in a marker.

In accordance with the present invention, a method of correlating a polymorphism in a NPC1L1 gene to the efficacy of a pharmaceutically active compound in a human subject is provided. The method comprises determining a polymorphism in an NPC1L1 gene of the human subject and determining the status of the human subject to which a pharmaceutically active compound was administered by reference to the single nucleotide polymorphism in the NPC1L1 gene.

Useful polymorphic nucleic acid molecules according to the present invention include those which will specifically hybridize to NPC1L1 sequences in the region of the C to A transversion that represents to the g.−18C>A SNP in the NPC₁L₁ promoter region. Typically such a polynucleotide is at least about 12 nucleotides in length and has a nucleotide sequence corresponding to the region of the C to A transversion at position 5,400 of the NPC1L1 sequence (SEQ ID NO: 1). One such representative polynucleotide is 5′ GGAGG(C)TGCCTT 3′ (SEQ ID NO:2), wherein the nucleotide base in the parentheses represents the “major” allele of polymorphic g.−18C>A site, i.e., a cytosine at position 5,400 of the NPC1L1 gene.

Provided nucleic acid molecules can be labeled according to any technique known in the art, such as with radiolabels, fluorescent labels, enzymatic labels, sequence tags, etc. According to another aspect of the invention, the nucleic acid molecules contain the C to A transversion at position 5,400 of SEQ ID NO: 1. Such molecules can be used as allele-specific oligonucleotide probes. Useful polynucleotides are at least about 12 nucleotides in length and include the polymorphic g.−18C>A site. One such representative polynucleotide is 5′ GGAGG(A)TGCCTT 3′ (SEQ ID NO:3), wherein the nucleotide base in the parentheses represents the “minor” allele of polymorphic g.−18C>A site, i.e., an adenine at position 5,400 of the NPC1L1 gene.

Tissue samples can be tested to determine which nucleotide base is present at a NPC1L1 polymorphic site. Suitable body samples for testing include those comprising DNA or RNA obtained from blood or any other cell sample from a subject containing DNA or RNA. For example, convenient samples include whole blood serum, semen, saliva, tears, fecal matter, urine, sweat, buccal matter, skin and hair. Somatic cells are preferred if determining the actual copy number of the marker variant. Nucleic acid samples may be prepared for analysis using any technique known to those skilled in the art. Preferably, such techniques result in the production of genomic DNA sufficiently pure for determining the genotype or haplotype pair for a desired set of polymorphic sites in the nucleic acid molecule. Such techniques may be found, for example, in Sambrook, et al., Molecular Cloning: A Laboratory Manual (Cold Spring Harbor Laboratory, New York) (2001).

In one embodiment of the invention, a pair of isolated oligonucleotide primers is provided for nucleic acid amplification of the NPC1L1 g.−18C>A polymorphism region, such as for example, SEQ ID NOS: 4 & 5, as disclosed in Example 1 herein. This set of primers is derived from the NPC1L1 gene, in particular, the 5′ UTR and exon 1 regions. Two appropriately positioned g.−18C>A amplification oligonucleotide primers are used to obtain sufficient nucleic acid material for sequencing of the g.−18C>A polymorphism region to determine which nucleotide base is present at position 5,400 of SEQ ID NO: 1. Similarly, other isolated oligonucleotide primers are disclosed in the Examples herein that can be used to amplify the NPC1L1 g.−133A>G, g. 1679C>G and g.28650A>G polymorphism regions.

In another embodiment of the invention isolated allele specific oligonucleotides (ASO) are provided, see for example, the ASOs described in Example 3 herein. Such ASOs can be used in the practice of a TaqMan Allelic Discrimination genotype assay as described by Livak ((1999) Genet. Anal., 14:143-9) and documents provided by Applied Biosystems (Foster City, Calif.) in conjunction with commercial reagents and custom allele discrimination genotype assay services. Sequences substantially similar thereto are also provided in accordance with the present invention. The ASOs are useful in identification of the presence or absence of each NPC1L1 polymorphism in a subject who has high cholesterol and is in need of treatment thereof. These unique NPC1L1 oligonucleotide primers are designed and produced based upon the base changes corresponding to the g.−133A>G, g.−18C>A, g.1679C>G and g.28650A>G, respectively. Other primers which can be used for primer hybridization are readily ascertainable to those of skill in the art based upon the disclosure herein of the NPC1L1 g.−133A>G, g.−18C>A, g.1679C>G and g.28650A>G polymorphisms.

The primers of the invention embrace oligonucleotides of sufficient length and appropriate sequence so as to provide initiation of polymerization on a significant number of nucleic acids in the polymorphic locus. Specifically, the term “primer” as used herein refers to a sequence comprising two or more deoxyribonucleotides or ribonucleotides, in some embodiments more than three, and other embodiments more than eight, and other embodiments more than twelve, and in still other embodiments at least about 20 nucleotides of the NPC1L1 gene wherein the DNA sequence contains each the polymorphic site corresponding to g.−133A>G, g.−18C>A, g.1679C>G and g.28650A>G, respectively. For example, in the case of NPC1L1-g.−18C>A, the C to A transversion at position 5,400 of SEQ ID NO: 1 is contained within the oligonucleotide. The allele including cystine (C) at position 5,400 of SEQ ID NO: 1 is referred to herein as the “5,400-major allele”. The allele including adenine (A) at position 5,400 of SEQ ID NO: 1 is referred to herein as the “5,400-minor allele”.

An oligonucleotide that distinguishes between the 5,400-major and the 5,400-minor alleles of the NPC1L1 gene, wherein the oligonucleotide hybridizes to a portion of the NPC1L1 gene that includes nucleotide 5,400 of a polynucleotide that corresponds to the NPC1L1 gene when the nucleotide 5,400 is cytosine, but does not hybridize with the portion of the NPC1L1 gene when the nucleotide 5,400 is adenine is also provided in accordance with the present invention. An oligonucleotide that distinguishes between the 5,400-major and the 5,400-minor alleles of the NPC1L1 gene, wherein the oligonucleotide hybridizes to a portion of the NPC1L1 gene that includes nucleotide 5,400 of the polynucleotide that corresponds to the NPC1L1 gene when nucleotide 5,400 is adenine, but does not hybridize with the portion of the NPC1L1 gene when nucleotide 5,400 is cytosine is also provided in accordance with the present invention. Such oligonucleotides are preferably between ten and thirty bases in length. Such oligonucleotides can optionally further comprises a detectable label. Based upon the information provided herein, similar ASOs can be designed for the major and minor alleles of NPC1L1 g.−133A>G, g. 1679C>G and g.28650A>G, respectively.

In some instances it is desirable to increase the specificity of an allele specific hybridization assay to prevent false positive detection. In such cases, a locked nucleic acid residue is placed at the 3′ end of the allele-specific primer (the base that matches the SNP allele) conferring increased mismatch discrimination between each respective NPC1L1-major and minor alleles. Appropriate high specificity NPC1L1 ASO primers containing locked nucleic acid residues may be obtained from Proligo LLC (Boulder, Colo.).

Environmental conditions conducive to polynucleotide synthesis based methods of amplification include the presence of nucleoside triphosphates and an agent for polymerization, such as DNA polymerase, and a suitable temperature and pH. The primer is preferably single stranded for maximum efficiency in amplification, but can be double stranded. If double stranded, the primer is first treated to separate its strands before being used to prepare extension products. The primer must be sufficiently long to prime the synthesis of extension products in the presence of the inducing agent for polymerization. The exact length of primer will depend on many factors, including temperature, buffer, and nucleotide composition. The oligonucleotide primer typically contains 12-20 or more nucleotides, although it can contain fewer nucleotides.

Primers of the invention are designed to be “substantially” complementary to each strand of the genomic locus to be amplified. This means that the primers must be sufficiently complementary to hybridize with their respective strands under conditions which allow the agent for polymerization to perform. In other words, the primers should have sufficient complementarity with the 5′ and 3′ sequences flanking the transition to hybridize therewith and permit amplification of the genomic locus.

Oligonucleotide primers of the invention are employed in the amplification method which is an enzymatic chain reaction that produces exponential quantities of polymorphic locus relative to the number of reaction steps involved. Typically, one primer is complementary to the negative (−) strand of the polymorphic locus and the other is complementary to the positive (+) strand. Annealing the primers to denatured nucleic acid followed by extension with an enzyme, such as the large fragment of DNA polymerase I (Kienow) and nucleotides, results in newly synthesized + and − strands containing the target polymorphic locus sequence. Because these newly synthesized sequences are also templates, repeated cycles of denaturing, primer annealing, and extension results in exponential production of the region (i.e., the target polymorphic locus sequence) defined by the primers. The product of the chain reaction is a discreet nucleic acid duplex with termini corresponding to the ends of the specific primers employed.

The oligonucleotide primers of the invention can be prepared using any suitable method, such as conventional phosphotriester and phosphodiester methods or automated embodiments thereof. In one such automated embodiment, diethylphosphoramidites are used as starting materials and can be synthesized as described by Beaucage et al., Tetrahedron Letters 22:1859-1862 (1981). One method for synthesizing oligonucleotides on a modified solid support is described in U.S. Pat. No. 4,458,066.

Any nucleic acid specimen, in purified or non-purified form, can be utilized as the starting nucleic acid or acids, providing it contains, or is suspected of containing, a nucleic acid sequence containing the polymorphic locus. Thus, the method can amplify, for example, DNA or RNA, including messenger RNA, wherein DNA or RNA can be single stranded or double stranded. In the event that RNA is to be used as a template, enzymes, and/or conditions optimal for reverse transcribing the template to DNA would be utilized. In addition, a DNA-RNA hybrid which contains one strand of each can be utilized. A mixture of nucleic acids can also be employed, or the nucleic acids produced in a previous amplification reaction herein, using the same or different primers can be so utilized. The specific nucleic acid sequence to be amplified, i.e., the polymorphic locus, can be a fraction of a larger molecule or can be present initially as a discrete molecule, so that the specific sequence constitutes the entire nucleic acid. It is not necessary that the sequence to be amplified be present initially in a pure form; it can be a minor fraction of a complex mixture, such as contained in whole human DNA.

DNA utilized herein can be extracted from a body sample, such as blood, tissue material (e.g., fat tissue), and the like by a variety of techniques such as that described by Maniatis et. al. in Molecular Cloning: A Laboratory Manual, Cold Spring Harbor, N.Y., p 280-281 (1982). If the extracted sample is impure, it can be treated before amplification with an amount of a reagent effective to open the cells, or animal cell membranes of the sample, and to expose and/or separate the strand(s) of the nucleic acid(s). This lysing and nucleic acid denaturing step to expose and separate the strands will allow amplification to occur much more readily.

The deoxyribonucleotide triphosphates dATP, dCTP, dGTP, and dTTP are added to the synthesis mixture, either separately or together with the primers, in adequate amounts and the resulting solution is heated to about 90-100 degree C. from about 1 to 10 minutes, preferably from 1 to 4 minutes. After this heating period, the solution is allowed to cool, which is preferable for the primer hybridization. To the cooled mixture is added an appropriate agent for effecting the primer extension reaction (called herein “agent for polymerization”), and the reaction is allowed to occur under conditions known in the art. The agent for polymerization can also be added together with the other reagents if it is heat stable. This synthesis (or amplification) reaction can occur at room temperature up to a temperature above which the agent for polymerization no longer functions. Thus, for example, if DNA polymerase is used as the agent, the temperature is generally no greater than about 40 degree C. Most conveniently the reaction occurs at room temperature.

The agent for polymerization can be any compound or system which will function to accomplish the synthesis of primer extension products, including enzymes. Suitable enzymes for this purpose include, but are not limited to, E. coli DNA polymerase I, Klenow fragment of E. coli DNA polymerase, polymerase mutants, reverse transcriptase, other enzymes, including heat-stable enzymes (i.e., those enzymes which perform primer extension after being subjected to temperatures sufficiently elevated to cause denaturation), such as Taq polymerase. A suitable enzyme will facilitate combination of the nucleotides in the proper manner to form the primer extension products which are complementary to each polymorphic locus nucleic acid strand. Generally, the synthesis will be initiated at the 3′ end of each primer and proceed in the 5′ direction along the template strand, until synthesis terminates, producing molecules of different lengths.

The newly synthesized strand and its complementary nucleic acid strand will form a double-stranded molecule under hybridizing conditions described herein and this hybrid is used in subsequent steps of the method. In the next step, the newly synthesized double-stranded molecule is subjected to denaturing conditions using any of the procedures described above to provide single-stranded molecules.

The steps of denaturing, annealing, and extension product synthesis can be repeated as often as needed to amplify the target polymorphic locus nucleic acid sequence to the extent necessary for detection. The amount of the specific nucleic acid sequence produced will accumulate in an exponential fashion. For additional methods see “PCR. A Practical Approach”, ILR Press, Eds. McPherson et al. (1992).

The amplification products can be detected by Southern blot analysis with or without using adioactive probes. In one such method, for example, a small sample of DNA containing a very low level of the nucleic acid sequence of the polymorphic locus is amplified, and analyzed via a Southern blotting technique or similarly, using dot blot analysis. The use of non-radioactive probes or labels is facilitated by the high level of the amplified signal. Alternatively, probes used to detect the amplified products can be directly or indirectly detectably labeled, for example, with a radioisotope, a fluorescent compound, a bioluminescent compound, a chemiluminescent compound, a metal chelator or an enzyme. Those of ordinary skill in the art will know of other suitable labels for binding to the probe, or will be able to ascertain such, using routine experimentation.

Sequences amplified by the methods of the invention can be further evaluated, detected, cloned, sequenced, and the like, either in solution or after binding to a solid support, by any method usually applied to the detection of a specific DNA sequence such as dideoxy sequencing, PCR, oligomer restriction (Saiki et al., Bio/Technology 3: 1008-1012 (1985), allele-specific oligonucleotide (ASO) probe analysis (Conner et al., Proc. Natl. Acad. Sci. U.S.A. 80:278 (1983), oligonucleotide ligation assays (OLAs) (Landgren et. al., Science 241:1007, 1988), and the like. Molecular techniques for DNA analysis have been reviewed (Landgren et. al., Science 242:229-237 (1988)).

Preferably, the method of amplifying is by PCR, as described herein and in U.S. Pat. Nos. 4,683,195; 4,683,202; and 4,965,188 each of which is hereby incorporated by reference; and as is commonly used by those of ordinary skill in the art. Alternative methods of amplification have been described and can also be employed as long as the NPC1L1 locus amplified by PCR using primers of the invention is similarly amplified by the alternative techniques. Such alternative amplification systems include but are not limited to self-sustained sequence replication, which begins with a short sequence of RNA of interest and a T7 promoter. Reverse transcriptase copies the RNA into cDNA and degrades the RNA, followed by reverse transcriptase polymerizing a second strand of DNA.

Another nucleic acid amplification technique is nucleic acid sequence-based amplification (NASBA™) which uses reverse transcription and T7 RNA polymerase and incorporates two primers to target its cycling scheme. NASBA™. amplification can begin with either DNA or RNA and finish with either, and amplifies to about 10⁸ copies within 60 to 90 minutes.

Alternatively, nucleic acid can be amplified by ligation activated transcription (LAT). LAT works from a single-stranded template with a single primer that is partially single-stranded and partially double-stranded. Amplification is initiated by ligating a cDNA to the promoter oligonucleotide and within a few hours, amplification is about 10⁸ to about 10⁹ fold. The Q-beta replicase system can be utilized by attaching an RNA sequence called MDV-1 to RNA complementary to a DNA sequence of interest. Upon mixing with a sample, the hybrid RNA finds its complement among the specimen's mRNAs and binds, activating the replicase to copy the tag-along sequence of interest.

Another nucleic acid amplification technique, ligase chain reaction (LCR), works by using two differently labeled halves of a sequence of interest which are covalently bonded by ligase in the presence of the contiguous sequence in a sample, forming a new target. The repair chain reaction (RCR) nucleic acid amplification technique uses two complementary and target-specific oligonucleotide probe pairs, thermostable polymerase and ligase, and DNA nucleotides to geometrically amplify targeted sequences. A two-base gap separates the oligo probe pairs, and the RCR fills and joins the gap, mimicking normal DNA repair.

Nucleic acid amplification by strand displacement activation (SDA) utilizes a short primer containing a recognition site for HincII with short overhang on the 5′ end which binds to target DNA. A DNA polymerase fills in the part of the primer opposite the overhang with sulfur-containing adenine analogs. HincII is added but only cuts the unmodified DNA strand. A DNA polymerase that lacks 5′ exonuclease activity enters at the site of the nick and begins to polymerize, displacing the initial primer strand downstream and building a new one which serves as more primer.

SDA produces greater than about a 10⁷-fold amplification in 2 hours at 37 degree C. Unlike PCR and LCR, SDA does not require instrumented temperature cycling. Another amplification system useful in the method of the invention is the Q-beta Replicase System. Although PCR is the preferred method of amplification if the invention, these other methods can also be used to amplify the NPC1L1-g.−18C>A locus as described in the method of the invention.

In another embodiment of the invention a method is provided for diagnosing or identifying a subject having a polymorphism associated with NPC1L1 antagonist therapy, comprising sequencing a target NPC1L1 nucleic acid of a sample from a subject by dideoxy sequencing, preferably following amplification of the target NPC1L1 nucleic acid.

In another embodiment of the invention a method is provided for identifying a subject that is more likely to exhibit a higher than average response to NPC1L1 antagonist therapy, comprising contacting a target nucleic acid of a sample from a subject with a reagent that detects the presence of the NPC1L1 polymorphism and detecting the reagent.

Another method comprises contacting a target nucleic acid of a sample from a subject with a reagent that detects the presence of the A to G transition associated with the NPC1L1-g.133A>G polymorphism, and detecting the transition. Another method comprises contacting a target nucleic acid of a sample from a subject with a reagent that detects the presence of the C to A transversion associated with the NPC1L1-g.−18C>A polymorphism, and detecting the transversion. Another method comprises contacting a target nucleic acid of a sample from a subject with a reagent that detects the presence of the G to T transversion associated with the NPC1L1-g.1680G>T polymorphism, and detecting the transversion. Another method comprises contacting a target nucleic acid of a sample from a subject with a reagent that detects the presence of the A to G transition associated with the NPC1L1-g.28650A>G polymorphism, and detecting the transition. A number of hybridization methods are well known to those skilled in the art. Many of them are useful in carrying out the invention.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those of ordinary skill in the art. Stringent temperature conditions will generally include temperatures in excess of 30 degree C., typically in excess of 37 degree C., and preferably in excess of 45 degree C. Stringent salt conditions will ordinarily be less than 1,000 mM, typically less than 500 mM, and preferably less than 200 mM. However, the combination of parameters is much more important than the measure of any single parameter. See, for example, Wetmur & Davidson, (1968) J. Mol. Biol. 31:349-70).

Accordingly, a nucleotide sequence of the present invention can be used for its ability to selectively form duplex molecules with complementary stretches of the NPC1L1 gene. Depending on the application envisioned, one employs varying conditions of hybridization to achieve varying degrees of selectivity of the probe toward the target sequence. For applications requiring a high degree of selectivity, one typically employs relatively stringent conditions to form the hybrids. For example, one selects relatively low salt and/or high temperature conditions, such as provided by 0.02M-0.15M salt at temperatures of about 50 degree C. to about 70 degree C. including particularly temperatures of about 55 degree C., about 60 degree C. and about 65 degree C. Such conditions are particularly selective, and tolerate little, if any, mismatch between the probe and the template or target strand.

In certain embodiments, it is advantageous to employ a nucleic acid sequence of the present invention in combination with an appropriate reagent, such as a label, for determining hybridization. A wide variety of appropriate indicator reagents are known in the art, including radioactive, enzymatic or other ligands, such as avidin/biotin, which are capable of giving a detectable signal. In some embodiments, one likely employs an enzyme tag such a urease, alkaline phosphatase or peroxidase, instead of radioactive or other environmentally undesirable reagents. In the case of enzyme tags, calorimetric indicator substrates are known which can be employed to provide a reagent visible to the human eye or spectrophotometrically, to identify specific hybridization with complementary nucleic acid-containing samples.

In general, it is envisioned that the hybridization probes described herein are useful both as reagents in solution hybridization as well as in embodiments employing a solid phase. In embodiments involving a solid phase, the sample containing test DNA (or RNA) is adsorbed or otherwise affixed to a selected matrix or surface. This fixed, single-stranded nucleic acid is then subjected to specific hybridization with selected probes under desired conditions. The selected conditions depend inter alia on the particular circumstances based on the particular criteria required (depending, for example, on the G+C contents, type of target nucleic acid, source of nucleic acid, size of hybridization probe, etc.). Following washing of the hybridized surface so as to remove nonspecifically bound probe molecules, specific hybridization is detected, or even quantified, via the label.

### IV. Other SNP Detection Methods

It will be appreciated that advances in the field of SNP detection have provided additional accurate, easy, and inexpensive large-scale genotyping techniques, such as dynamic allele-specific hybridization (DASH) (Howell, et al., (1999), Nat. Biotechnol., 17:87-8), microplate array diagonal gel electrophoresis (MADGE) (Day, et al., (1995) Biotechniques, 19:830-5), the TaqMan system (Holland, et al., (1991), Proc Natl Acad Sci USA. 88:7276-80), as well as various DNA “microarray” technologies such as the GENECHIP® microarrays (e.g., Affymetrix SNP arrays) which are disclosed in U.S. Pat. No. 6,300,063 to Lipshutz, et al. 2001, Genetic Bit Analysis (GBA®) which is described by Goelet, et al., (PCT Appl. No. 92/15712), peptide nucleic acid (PNA), (Ren, et al., (2004) Nucleic Acids Res. 32:e42) and locked nucleic acids (LNA) probes, (Latorra, et al., (2003) Hum. Mutat., 22:79-85), Molecular Beacons (Abravaya, et al., (2003) Clin. Chem. Lab. Med., 41:468-74), intercalating dye (Germer and Higuchi, Genome Res., 9:72-78 (1999), FRET primers (Solinas et al., (2001) Nucleic Acids Res. 29: E96), AlphaScreen (Beaudet, et al., (2001) Genome Res., 11:600-8), SNPstream (Bell et al., (2002) Biotechniques. Suppl.:70-2, 74, 76-7), Multiplex minisequencing (Curcio, et al., (2002) Electrophoresis, 23:1467-72), SnaPshot (Turner, et al., (2002) Hum. Immunol., 63:508-13), MassEXTEND (Cashman, et al., (2001) Drug Metab. Dispos., 29:1629-37), GOOD assay (Sauer and Gut (2003) Rapid Commun. Mass. Spectrom., 17:1265-72), Microarray minisequencing (Liljedahl, et al., (2003) Pharmacogenetics, 13:7-17), arrayed primer extension (APEX) (Tonisson, et al., (2000) Clin. Chem. Lab. Med., 38:165-70), Microarray primer extension (O'Meara, et al., (2002) Nucleic Acids Res., 30: e75), Tag arrays (Fan, et al., (2000) Genome Res., 10:853-60), Template-directed incorporation (TDI) (Akula, et al., (2002) Biotechniques, 32:1072-8), fluorescence polarization (Kwok, (2002) Human Mutation, 19:315-23), Colorimetric oligonucleotide ligation assay (OLA), Nickerson, et al., (1990), Proc. Natl. Acad. Sci. USA, 87:8923-7), Sequence-coded OLA (Gasparini, et al., (1999) J. Med. Screen, 6:67-9), Microarray ligation, Ligase chain reaction, Padlock probes, Rolling circle amplification, Invader assay (reviewed in Shi, (2001) Clin Chem., 47:164-72), coded microspheres (Rao, et al., (2003) Nucleic Acids Res. 31: e66) and MassArray (Leushner and Chiu, (2000) Mol. Diagn., 5:341-80). Many of the above-referenced methods are also discussed in an article reviewing methods for genotyping single nucleotide polymorphisms (Kwak, (2001) Annu. Rev. Genomics Hum. Genet., 2:235-58).

### V. Association of Genotype Markers with Responsiveness to a Cholesterol Treatment Drug

In the context of the present invention, an association between single nucleotide polymorphisms and haplotypes in the NPC1L1 gene and responsiveness to the cholesterol treatment drug ezetimibe was discovered. Similar methods to those described herein may be used to find associations between other NPC1L1 polymorphisms and the efficacy of other agents that modify NPC1L1 function.

In order to investigate and identify a genetic origin to ezetimibe-associated lowering of cholesterol levels, an association analysis was conducted. This approach comprised: identifying polymorphic markers in the NPC1L1 gene encoding the target of ezetimibe, and conducting association studies to identify polymorphic marker alleles or haplotypes associated with reduced cholesterol levels upon treatment with ezetimibe.

Statistical association analysis is performed for a population of individuals who have been tested for the presence or absence of a phenotypic trait of interest or on whom a measurement of a quantitative phenotype was assessed and for polymorphic markers sets. To perform such analysis, the presence or absence of a set of polymorphisms (i.e., a polymorphic set) is determined for a set of the individuals; some of whom exhibit a particular trait, and some of whom exhibit lack of the trait. Otherwise, these individuals are scored for a quantitative phenotype if that is the measurement of interest. Association analysis is used to describe the degree to which one variable is linearly related to another. Typically, association analysis is tested in a regression analysis framework to measure how well the least squares line fits the data. It can also be tested with chi-square statistics or equivalent in the context of categorical traits and tables.

The alleles of each polymorphism of the set are then reviewed to determine whether the presence or absence of a particular allele is associated with the trait of interest. Correlation can be performed by standard statistical methods such as a chi squared test and statistically significant correlations between polymorphic form(s) and phenotypic characteristics are noted. For example, it might be found that the presence of allele A1 at polymorphism A occurs more often with a disease related phenotype, such as high cholesterol level, than it does with a normal phenotype, such as normal cholesterol level. As a further example, it might be found that the combined presence of allele A2 at polymorphism A and allele B1 at polymorphism B is associated with an increased average response to a drug treatment as compared to other allele combinations at polymorphism sites A and B.

Genetic association analysis is typically carried out within a study population of human subjects that is split into at least two groups; those receiving the pharmaceutically active compound or drug and those who are not. The status of each group is measured by reference to an appropriate measure of response to the pharmaceutically active compound, such as, for example, plasma cholesterol lowering. In addition, a nucleic acid sample is taken from each human subject in each group. However, it should be noted that it is not necessary that the individuals in no drug group, i.e., the placebo group, be genotyped. Individual SNPs, haplotypes, and haplotype combinations are then tested as principal explanatory variables in statistical analyses of the data, using for example a statistical software program.

In one embodiment, the analysis technique is the PROC GLM tool in SAS/STAT® Software (SAS Institute, Inc., Cary, N.C.) and involves the comparison of means between groups, taking into account for some of the models variation explained by additional continuous measurements. A continuous response, for example, “percent change from baseline LDL-C”, is measured and classification variables (here the genotypic categories) are scored. The variation in the response is explained as being due to effects in the classification, with random error accounting for the remaining variation (effects that are not identified a priori as important in explaining the continuous outcome). The statistical theory of these techniques is well established, and the tools are commonly used in applied statistical problems (see for example, Fisher, R. A. (1942), The design of Experiments, 3d edition, Edinburgh: Oliver and Boyd). In particular, the SAS software program has implemented many of these statistical methods in several of its procedures. In this regard, the SAS implemented tools PROC GLM, PROC FREQ, and PROC HAPLOTYPES are particularly useful in association analysis and in the identification of haplotypes which can then be used in the association analyses. Other software and statistical methods may be used in the practice of association analysis and are well known in the art. Baseline parameters such as drug responsive phenotype measurements, for example LDL-C level, sex, age, and race can be investigated to determine if they give rise to significant effects. In other embodiments, association analysis is performed using the more general “General Linear Model” tool: PROC GLM. The SAS PROC GLM tool allows for variation explained by another continuous observed variable (for instance here “baseline LDL-C levels”) to be taken into account in the analyses of the percent change from baseline LDL-C outcome. Further details regarding association analysis are provided in Example 3 herein.

### VI. Diagnostic Kits

The invention kits comprise components useful in any of the methods described herein, including for example, hybridization probes, restriction enzymes (e.g., for RFLP analysis), or allele-specific oligonucleotides, but probes or ASOs comprising at least one genetic marker included in the SNPs or haplotypes described herein, means for amplification of nucleic acids comprising NPC1L1 containing the SNP or haplotype sequences and means for analyzing the nucleic acid sequence of NPC1L1. Additionally, kits can provide reagents for assays to be used in combination with the methods of the present invention, e.g., reagents for use in determining one or more of: total cholesterol, non-high density lipid-cholesterol (nonHDL-c), low density lipid-cholesterol (LDL-c), LDL-c:HDL-c ratio, triglycerides, blood hemoglobin A1c, and apolipoprotein B.

Kits (e.g., reagent kits) useful in the methods of diagnosis comprise components useful in any of the methods described herein, including for example, hybridization probes or primers as described herein (e.g., labeled probes or primers), reagents for detection of labeled molecules, restriction enzymes (e.g., for RFLP analysis), allele-specific oligonucleotides, means for amplification of nucleic acids comprising NPC1L1, means for analyzing the nucleic acid sequence of a NPC1L1 nucleic acid, instructions for use, etc.

A kit in accordance with the present invention can further comprise solutions, buffers or other reagents for extracting a nucleic acid sample from a biological sample obtained from a subject. By way of particular example, a suitable lysis buffer for the tissue or cells along with a suspension of glass beads for capturing the nucleic acid sample and an elution buffer for eluting the nucleic acid sample off of the glass beads comprise a reagent for extracting a nucleic acid sample from a biological sample obtained from a subject.

Other examples include commercially available extraction kits, such as the GENOMIC ISOLATION KIT A.S.A.P.™ (Boehringer Mannheim, Indianapolis, Ind.), Genomic DNA Isolation System (GIBCO BRL, Gaithersburg, Md.), ELU-QUIK.®. DNA Purification Kit (Schleicher & Schuell, Keene, N.H.), DNA Extraction Kit (Stratagene, La Jolla, Calif.), TURBOGEN.™. Isolation Kit (Invitrogen, San Diego, Calif.), and the like. Use of these kits according to the manufacturer's instructions is generally acceptable for purification of DNA prior to practicing the methods of the present invention.

In one embodiment, the invention is a kit for assaying a sample from a subject to predict responsiveness of a subject to a drug affecting NPC1L1 function in a subject, wherein the kit comprises one or more reagents for detecting an ezetimibe response predictive SNP or haplotype associated with the NPC1L1 gene. In particular embodiments, the kit can comprise, e.g., at least one contiguous nucleotide sequence that is completely complementary to a region comprising at least one of the ezetimibe response predictive SNPs or haplotypes, such as g.−18C>A, one or more nucleic acids that are capable of detecting one or more of the ezetimibe response predictive SNP or haplotype. Such nucleic acids (e.g., oligonucleotide primers) can be designed using portions of the nucleic acids flanking SNPs that are indicative of ezetimibe responsiveness or the responsiveness of any other compound that affects NPC1L1 cholesterol related function. Such nucleic acids (e.g., oligonucleotide primers) are designed to amplify regions of the NPC1L1 nucleic acid (and/or flanking sequences) that are associated with an ezetimibe response predictive SNP or haplotype for a cholesterol-associated condition. In another embodiment, the kit comprises one or more labeled nucleic acids capable of detecting one or more the ezetimibe response predictive SNP or haplotype associated with the NPC1L1 gene and reagents for detection of the label. Suitable labels include, e.g., a radioisotope, a fluorescent label, an enzyme label, an enzyme co-factor label, a magnetic label, a spin label, an epitope label. Suitable ezetimibe response predictive SNPs include g.−18C>A and g.1679C>G and suitable haplotypes include [A(−133), A(−18), G(1679) and [G(−133), C(−18), C(1679)].

In some embodiments, the set of oligonucleotides in the kit are allele-specific oligonucleotides. As used herein, the term allele-specific oligonucleotide (ASO) means an oligonucleotide that is able, under sufficiently stringent conditions, to hybridize specifically to one allele of a PS, at a target region containing the PS while not hybridizing to the same region containing a different allele. Allele-specificity will depend upon a variety of readily optimized stringency conditions, including salt and formamide concentrations, as well as temperatures for both the hybridization and washing steps. Examples of hybridization and washing conditions typically used for ASO probes and primers are found in Kogan et al., “Genetic Prediction of Hemophilia A” in PCR PROTOCOLS, A GUIDE TO METHODS AND APPLICATIONS, Academic Press, 1990, and Ruaño et al., Proc. Natl. Acad. Sci. USA 87:6296-300 (1990).

Typically, an ASO will be perfectly complementary to one allele while containing a single mismatch for another allele. In ASO probes, the single mismatch is preferably within a central position of the oligonucleotide probe as it aligns with the polymorphic site in the target region (e.g., about the 8^{th} or 9^{th} position in an ASO probe of 16 bases, and the 10^{th} or 11^{th} position in an ASO probe of 20 bases). The single mismatch in ASO primers may be located at the 3′ terminal nucleotide, but is preferably located at the 3′ penultimate nucleotide. ASO probes and primers hybridizing to either the coding or noncoding strand are contemplated by the invention. Primers hybridizing to the noncoding strand are referred to herein as forward primers, and primers hybridizing to the coding strand are referred to herein as reverse primers.

In other embodiments, the kit comprises a pair of allele-specific oligonucleotides for each PS to be assayed, with one member of the pair being specific for one allele and the other member being specific for the other allele. In such embodiments, the oligonucleotides in the pair may have different lengths or have different detectable labels to allow the user of the kit to determine which allele-specific oligonucleotide has specifically hybridized to the target region, and thus determine which allele is present in the individual at the assayed PS.

Exemplary ASO probes for detecting the alleles at each PS in the NPC1L1 markers shown in Table 1 comprise the ASO probe sequences listed in Tables 2A and 2B, or their complements. Tables 2A and 2B also list sequences comprising preferred ASO forward and reverse primers for genotyping these NPC1L1 PS by allele-specific PCR.

In still other embodiments, the oligonucleotides in the kit are primer-extension oligonucleotides for use in polymerase-mediated extension methods. Termination mixes for polymerase-mediated extension from any of these oligonucleotides are chosen to terminate extension of the oligonucleotide at the PS of interest, or one base thereafter, depending on the alternative nucleotides present at the PS. Tables 2A and 2B also list sequences comprising preferred forward and reverse primer-extension oligonucleotides for detecting the alleles at each PS in the NPC1L1 markers shown in Table 1.

TABLE 2A

Exemplary oligonucleotides for detecting an
NPC1L1 marker of a health risk level of LDL-C.

| | g.28650A > G | |
|---|---|---|
| Genotyping Oligo | Sequence | SEQ ID NO |

| | | |
|---|---|---|
| ASO Probe | CAGAAGCRTGAACTG | 156 |
| ASO Forward Primer | GCTCTCCAGAAGCRT | 157 |
| ASO Reverse Primer | CCACTGCAGTTCAYG | 158 |
| Forward Extension Primer | CAGCTCTCCAGAAGC | 159 |
| Reverse Extension Primer | CTCCACTGCAGTTCA | 160 |

TABLE 2B

Exemplary oligonucleotides for detecting NPC1L1
markers of increased ezetimibe response.

| Genotyping | Sequence in Genotyping Oligo | | |
|---|---|---|---|
| Oligo | g.−133A > G | g.−18C > A | g.1679C > G |

| | | | |
|---|---|---|---|
| ASO Probe | AGGGCTCRGCCTCAT | CCGCTGAMCCCTTCC | AGGCCCTSGACTCCA |
| | SEQ ID NO:161 | SEQ ID NO:166 | SEQ ID NO:171 |
| ASO Forward | ACCAGCAGGGCTCRG | GGCTCCCCGCTGAMC | GCCCCCAGGCCCTSG |
| Primer | SEQ ID NO:162 | SEQ ID NO:167 | SEQ ID NO:172 |
| ASO Reverse | GGACCAATGAGGCYG | GGTCTGGGAAGGGKT | AGAAGGTGGAGTCSA |
| Primer | SEQ ID NO:163 | SEQ ID NO:168 | SEQ ID NO:173 |
| Forward | TAACCAGCAGGGCTC | CTGGCTCCCCGCTGA | CCGCCCCCAGGCCCT |
| Extension | SEQ ID NO:164 | SEQ ID NO:169 | SEQ ID NO:174 |
| Primer | | | |
| Reverse | AGGGACCAATGAGGC | CAGGTCTGGGAAGGG | GTAGAAGGTGGAGTC |
| Extension | SEQ ID NO:165 | SEQ ID NO:170 | SEQ ID NO:175 |
| Primer | | | |

The sequences in Tables 2A and 2B use commonly accepted symbols for the indicated alternative alleles at each PS to indicate that the probe or primer contains one of the two alternative alleles at the corresponding oligonucleotide position. These symbols are: K=G or T/U; M=A or C; R=G or A; S=G or C and Y=T/U or C (World Intellectual Property Organization Handbook on Industrial Property Information and Documentation, Standard ST.25 1998)

In still further embodiments, the oligonucleotides in the kit are designed for performing allelic discrimination assays on the TaqMan System. Such assays typically employ a pair of PCR primers, a fluorescently labeled probe for detecting the major allele, and a different fluorescently labeled probe for detecting the minor allele. Table 3 in the Examples lists preferred oligonucleotides for assaying the SNPs in the NPC1L1 markers using the TaqMan System.

Methods and kits of the invention include the following specific embodiments.

1. A method of testing a human individual for susceptibility for a health risk level of plasma cholesterol, which comprises: detecting the presence or absence of guanine at position 34,067 of SEQ ID NO: 1 in the individual's Niemann Pick C1-Like 1 (NPC1L1) gene; and generating a test report for the individual which indicates whether guanine is present or absent in the individual. In some embodiments, the test report is a written document prepared by the testing laboratory and sent to the individual or the individual's physician as a hard copy or via electronic mail. In other embodiments, the test report is generated by a computer program and displayed on a video monitor in the physician's office. The test report may also comprise an oral transmission of the test results directly to the patient or the patient's physician or an authorized employee in the physician's office. Similarly, the test report may comprise a record of the test results that the physician makes in the patient's file. In a preferred embodiment, if guanine is present, then the test report further indicates that the individual tested positive for a polymorphism associated with a health risk level of plasma cholesterol. In another preferred embodiment, if guanine is absent, then the test report further indicates that the individual tested negative for a polymorphism associated with a health risk level of plasma cholesterol. The test report may be sent to a physician designated by the individual or to the individual whose NPC1L1 gene is being tested. In particularly preferred embodiments, the individual is self-identified as a Caucasian.

2. A method of testing a human individual for the presence or absence of a marker in the Niemann Pick C1-Like 1 (NPC1L1) gene that is associated with an increased LDL-C response to an NPC1L1 antagonist, which comprises: determining, for a biological sample obtained from the individual, the copy number of an allele in the NPC1L1 gene that is associated with the LDL-C response; using the determined copy number to assign to the individual the presence or absence of the genetic marker; and generating a test report which indicates whether the NPC1L1 marker is present or absent in the individual. Preferably, if the presence of the NPC1L1 marker is assigned to the individual, the test report further indicates that the individual is likely to exhibit a higher than average LDL-C response to the NPC1L1 antagonist, and if the absence of the NPC1L1 marker is assigned to the individual, the test report further indicates that the individual is likely to exhibit an average LDL-C response to the NPC1L1 antagonist. The test report may be sent to a physician designated by the individual or to the individual whose NPC1L1 gene is being tested. In some particularly preferred embodiments, the individual is self-identified as a Caucasian. In other particularly preferred embodiments, the NPC1L1 antagonist is ezetimibe.
a. In some preferred embodiments, the allele comprises: (i) adenine at position 5,400 of SEQ ID NO: 1; (ii) guanine at position 7,096 of SEQ ID NO: 1; or (iii) adenine, adenine and guanine at positions 5,285, 5,400 and 7,096 of SEQ ID NO:1, respectively. If the determined copy number for the allele is 1 or 2, then the presence of the NPC1L1 marker is assigned to the individual, and if the determined copy number for the allele is 0, then the absence of the NPC1L1 marker is assigned to the individual.
b. In other preferred embodiments, the allele comprises guanine, cytosine and cytosine at positions 5,285, 5,400 and 7,096 of SEQ ID NO: 1, respectively, and if the determined copy number for the allele is 0, then the presence of the NPC1L1 marker is assigned to the individual, and if the determined copy number for the allele is 1 or 2, then the absence of the NPC1L1 marker is assigned to the individual.
c. Determining the copy number for the haplotype alleles in (a) or (b) of this Section A.2 preferably comprises obtaining the individual's genotype for positions 5,285, 5,400 and 7,096 of SEQ ID NO: 1 and inputting the genotype into a computer that executes a computer program to infer the individual's haplotype pair for these positions.
3. A method of predicting the LDL-C response of a human individual to an antagonist of the Niemann pick C1-Like 1 (NPC1L1) gene, which comprises: determining the presence or absence in the individual of an NPC1L1 marker that is associated with an increased LDL-C response to the antagonist; and making a prediction based on the results of the determining step; wherein if the NPC1L1 marker is present, the prediction is that the individual is likely to exhibit a higher than average LDL-C response to the NPC1L1 antagonist, and if the NPC1L1 marker is absent, then the prediction is that the individual is likely to exhibit an average LDL-C response to the NPC1L1 antagonist. The prediction may be reported to the individual or to a physician treating the individual. In some particularly preferred embodiments, the individual is self-identified as a Caucasian. In other particularly preferred embodiments, the NPC1L1 antagonist is ezetimibe.
a. In some preferred embodiments, the NPC1L1 marker comprises: (i) 1 or 2 copies of adenine at position 5,400 of SEQ ID NO: 1, 1 or 2 copies of guanine at position 7,096 of SEQ ID NO: 1; or (iii) 1 or 2 copies of adenine, adeninc and guanine at positions 5,285, 5,400 and 7,096 of SEQ ID NO: 1, respectively.
b. In other preferred embodiments, the NPC1L1 marker comprises 0 copies of guanine, cytosine and cytosine at positions 5,285, 5,400 and 7,096 of SEQ ID NO: 1, respectively.
c. Determining the presence of absence of the NPC1L1 marker defined in (a) or (b) of this Section A.3 preferably comprises ordering a test to be performed by a testing laboratory; and receiving from the laboratory a test report that indicates whether the NPC1L1 marker is present or absent in the individual.
   (i) Preferably, the test comprises determining, for a biological samples obtained from the individual, the individual's genotype for positions 5,285, 5,400 and 7,096 of SEQ ID NO: 1; inferring the individual's haplotype pair for these positions from the determined genotype; and assigning to the individual the presence or absence of the NPC1L1 marker from the inferred haplotype pair, wherein the presence of the NPC1L1 marker is assigned to the individual if the inferred haplotype pair contains at least one copy of adenine, adenine and guanine or zero copies of guanine, cytosine and cytosine, and wherein the absence of the NPC1L1 marker is assigned to the individual if the inferred haplotype pair contains zero copies of adenine, adenine and guanine or at least one copy of guanine, cytosine and cytosine. The haplotype pair is preferably inferred by inputting the determined genotype into a computer that executes a computer program that compares the determined genotype to a set of reference haplotype pairs for positions 5,285, 5,400 and 7,096 of SEQ ID NO: 1 and assigns to the determined genotype the reference haplotype pair from the set that is most likely to exist in the individual.

4. A kit for detecting a genetic marker in the human Niemann pick C1-Like 1 (NPC1L1) gene that is associated with an increased LDL-C response to an NPC1L1 antagonist, the kit comprising a set of oligonucleotides designed for identifying each of the alleles at each polymorphic site (PS) in the NPC1L1 marker. Preferably, the NPC1L1 antagonist is ezetimibe.
a. In some preferred embodiments, the NPC1L1 marker comprises (i) a PS at position 5,285 of SEQ ID NO: 1.
b. In other preferred embodiments, the NPC1L1 marker further comprises a PS at each of positions 5,400 and 7,096 of SEQ ID NO:1.
   (i) This kit preferably further comprises a manual with instructions for performing one or more reactions on a human nucleic acid sample to determine the genotype of the sample at positions 5,285, 5,400 and 7,096 of SEQ ID NO:1. More preferably, the kit further comprises a computer-usable medium having computer-readable program code stored thereon, for causing a computer to execute a process that uses the determined genotype to assign to the sample a haplotype pair for positions 5,285, 5,400 and 7,096 of SEQ ID NO:1.
   (ii) In one particularly preferred embodiment, the set of oligonucleotides comprises an allele-specific oligonucleotide (ASO) probe for each of the adenine and guanine alleles at position 5,285, each of the cytosine and adenine alleles at position 5,400 and each of the cytosine and guanine alleles at position 7,096. Preferably, the set of oligonucleotides comprises a first ASO probe which comprises SEQ ID NO: 161, a second ASO probe which comprises SEQ ID NO: 166, and a third ASO probe which comprises SEQ ID NO: 171.
   (iii) In a second particularly preferred embodiment, the set of oligonucleotides comprises a primer-extension oligonucleotide for each PS. Preferably, the set of oligonucleotides comprises a first primer extension oligo comprising SEQ ID NO: 164, a second primer extension oligo comprising SEQ ID NO: 165, a third primer extension oligo comprising SEQ ID NO: 169, a fourth primer extension oligo comprising SEQ ID NO: 170, a fifth primer extension oligo comprising SEQ ID NO: 174, and a sixth primer extension oligo comprising SEQ ID NO: 175.
   (iv) In a third particularly preferred embodiment, the set of oligonucleotides comprises a first pair of PCR primers and a first pair of ASO probes designed for genotyping position 5,285, a second pair of PCR primers and a second pair of ASO probes designed for genotyping position 5,400 and a third pair of PCR primers and a third pair of ASO probes designed for genotyping position 7,096 of SEQ ID NO: 1. Preferably, the first pair of PCR primers consists of an oligonucleotide comprising SEQ ID NO:104 and an oligonucleotide comprising SEQ ID NO: 105, the first pair of probe sequences consists of an oligonucleotide comprising SEQ ID NO:106 and an oligonucleotide comprising SEQ ID NO: 107, the second pair of PCR primers consists of an oligonucleotide comprising SEQ ID NO: 108 and an oligonucleotide comprising SEQ ID NO: 109, the second pair of probe sequences consists of an oligonucleotide comprising SEQ ID NO: 110 and an oligonucleotide comprising SEQ ID NO: 111, the third pair of PCR primers consists of an oligonucleotide comprising SEQ D) NO: 112 and an oligonucleotide comprising SEQ ID NO: 113, and the third pair of probe sequences consists of an oligonucleotide comprising SEQ D) NO: 114 and an oligonucleotide comprising SEQ ID NO: 115.

5. A kit for detecting a genetic marker in the human Niemann pick C1-Like 1 (NPC1L1) gene that is associated with a health risk level of LDL-C, the kit comprising a set of oligonucleotides designed for identifying each of the alleles at position 28,650 of SEQ ID NO: 1.
a. In one preferred embodiment, the set of oligonucleotides comprises an allele-specific oligonucleotide (ASO) probe for each of the adenine and guanine alleles at position 28,650. Preferably, the set of oligonucleotides comprises a first ASO probe comprising SEQ ID NO: 156, wherein R=adenine and a second ASO probe comprising SEQ ID NO: 156, wherein R=guanine.
b. In a second preferred embodiment, the set of oligonucleotides comprises a primer extension oligonucleotide for each of the adenine and guanine alleles at position 28,650. Preferably, the set of oligonucleotides comprises a first primer comprising SEQ ID NO: 159 and a second primer comprising SEQ ID NO: 160.
c. In a third preferred embodiment, the set of oligonucleotides comprises a pair of PCR primers and a pair of ASO probes designed for genotyping position 28,650. Preferably, the pair of PCR primers consists of an oligonucleotide comprising SEQ ID NO: 152 and an oligonucleotide comprising SEQ ID NO:153, and the pair of ASO probes consists of an oligonucleotide comprising SEQ ID NO: 154 and an oligonucleotide comprising SEQ ID NO: 155.

As mentioned above, cholesterol levels are determined by a variety of genetic and environmental factors. Individuals having high cholesterol levels have increased risk for developing atherosclerosis, which is the predominant underlying factor in vascular disorders such as coronary artery disease, acute coronary syndrome, aortic aneurysm, arterial disease of the lower extremities and cerebrovasular disease. Cholesterol management therefore relies on early and regular use of drugs that lower cholesterol thereby preventing atherosclerosis. As a consequence, there is a need for efficient and safe therapeutic opportunities for patients with high cholesterol. There are now two main categories of cholesterol drugs-statins, which inhibit cholesterol biosynthesis and ezetimibe, which inhibits intestinal absorption of cholesterol. Not all individuals show the same response to either statins or ezetimibe, or a combination thereof. Therefore, in one embodiment, the kits of the present invention are used to identify individuals that will exhibit a beneficial response to one or more drug. In other embodiments, the kits are used in the practice of a clinical trial.

In one aspect, the invention provides a method for stratifying a human subject in a subgroup of a clinical trial of a therapy for the treatment of high cholesterol or a disease associated with high cholesterol. The inventive method includes determining the genotype of a NPC1L1 gene of the human subject at nucleotide position 5,400 of SEQ ID NO: 1. The subject is stratified into one or more subgroups of the clinical trial based upon the nucleotide base present at position 5,400 of SEQ ID NO: 1 of the NPC1L1 gene. In others embodiments, this method is practiced based upon a determination of the genotype at one or more NPC1L1 nucleotide position selected from the group consisting of position 5,285, position 5,400, position 7,096, and position 34,067.

In another aspect, a method is provided for selecting an individual for inclusion in a clinical trial of a high cholesterol drug or treatment. The method includes obtaining a nucleic acid sample from an individual; determining the identity of a polymorphic base at a NPC1L1-related single nucleotide polymorphism in the nucleic acid sample, wherein the identity of the polymorphic base determines the genotype of the individual at the NPC1L1-related single nucleotide-polymorphism and, wherein the NPC1L1-related single nucleotide polymorphism is positioned in SEQ ID NO: 1; determining whether the NPC1L1-related single nucleotide polymorphism is associated with a higher than average response or a lower than average response to the drug or treatment as compared to a persons not having the identified polymorphism; and including the individual in the clinical trial if the nucleic acid sample contains at least one single nucleotide polymorphism which is associated with a higher than average response to the drug or treatment, or if the nucleic acid sample lacks at least one single nucleotide polymorphism associated with a lower than average response to the drug or treatment.

### VI. Treatment Regimes

The NPC1L1 markers of the invention that are associated with an increased ezetimibe response are useful for helping physicians predict the effectiveness of a particular treatment regimen for patient with an elevated LDL-C. The marker information would be used in concert with other patient information such as the existing level of LDL-C and the desired level of LDL-C.

Examples of possible patient regimes that could be favored based on NPC1L1 marker information include use of a lower statin dose (or other LDL-C lowering drug) and/or higher NPC1L1 antagonist dose. For example, depending upon the desired LDL-C lowering, in some cases where the patient tests positive for a drug response markers, the physician may decide to prescribe using an NPC1L1 antagonist as a monotherapy, or using a lower statin level in conjugation with an NPC1L1 antagonist. Alternatively, if the maker is not present the physician may consider using a higher dose of NPC1L1 antagonist and/or a longer treatment regime involving NPC1L1 antagonist.

The treatment algorithm devised by the physician for a particular patient will typically incorporate a consideration of other patient-specific factors, including the presence of other risk factors for vascular disease, symptoms of vascular disease and the patient's tolerance for therapy with the NPC1L1 antagonist and other cholesterol lowering drugs. For example, in some embodiments, the patient has a health risk level of plasma LDL-C. In other embodiments, the patient has tested positive for a genetic marker that is correlated with a health risk level of plasma LDL-C, and may also have other risk factors for LDL-C. In still further embodiments, the patient has a health risk level of cholesterol after prior therapy with another cholesterol lowering drug. Preferred cholesterol lowering drugs that could be prescribed with an NPC1L1 antagonist such as ezetimibe include statins, which are a class of compounds that inhibit HMG CoA reductase activity.

Exemplary statins include, but are not limited to, mevastatin and related compounds as disclosed in U.S. Pat. No. 3,983,140, lovastatin (mevinolin) and related compounds as disclosed in U.S. Pat. No. 4,231,938, pravastatin and related compounds such as disclosed in U.S. Pat. No. 4,346,227, simvastatin and related compounds as disclosed in U.S. Pat. Nos. 4,448,784 and 4,450,171. Other HMG CoA reductase inhibitors which may be employed herein include, but are not limited to, fluvastatin, disclosed in U.S. Pat. No. 5,354,772, cerivastatin disclosed in U.S. Pat. Nos. 5,006,530 and 5,177,080, atorvastatin disclosed in U.S. Pat. Nos. 4,681,893, 5,273,995, 5,385,929 and 5,686,104, pitavastatin (Nissan/Sankyo's nisvastatin (Ne-104) or itavastatin), disclosed in U.S. Pat. No. 5,011,930, Shionogi-AstratZeneca rosuvastatin (visastatin (ZD-4522)) disclosed in U.S. Pat. No. 5,260,440, and related statin compounds disclosed in U.S. Pat. No. 5,753,675, pyrazole analogs of mevalonolactone derivatives as disclosed in U.S. Pat. No. 4,613,610, indene analogs of mevalonolactone derivatives as disclosed in PCT application WO 86/03488, 6-[2-(substituted-pyrrol-1-yl)-alkyl)pyran-2-ones and derivatives thereof as disclosed in U.S. Pat. No. 4,647,576, Searle's SC-45355 (a 3-substituted pentanedioic acid derivative) dichloroacetate, imidazole analogs of mevalonolactone as disclosed in PCT application WO 86/07054, 3-carboxy-2-hydroxy-propane-phosphonic acid derivatives as disclosed in French Patent No. 2,596,393, 2,3-disubstituted pyrrole, furan and thiophene derivatives as disclosed in European Patent Application No. 0221025, naphthyl analogs of mevalonolactone as disclosed in U.S. Pat. No. 4,686,237, octahydronaphthalenes such as disclosed in U.S. Pat. No. 4,499,289, keto analogs of mevinolin (lovastatin) as disclosed in European Patent Application No. 0,142,146 A2, and quinoline and pyridine derivatives disclosed in U.S. Pat. Nos. 5,506,219 and 5,691,322.

In another embodiment of the method the high cholesterol therapy is treatment with a compound that binds to NPC1L1 protein. Typically, treatment with the NPC1L1-binding compound results in a reduction in the level of low density lipid cholesterol in subjects receiving treatment. In yet another embodiment of the inventive method, the high cholesterol therapy is a dual therapy combining statin drug treatment with a NPC1L1 mediated drug treatment, such as ezetimibe.

### VII. Exemplary NPC1L1 Antagonists

Some aspects of the invention are useful to access the responsiveness of a subject to drugs that affect the activity of NPC1L1, such as, for example, drugs that disrupt absorption of intestinal cholesterol mediated by NPC1L1 either directly or indirectly. In one specific embodiment of the invention the NPC1L1 antagonist is ezetimibe. Ezetimibe is in a class of lipid-lowering compounds, known as azetidinones, that selectively inhibits the intestinal absorption of cholesterol and related phytosterols. The chemical name of ezetimibe is 1-(4-fluorophenyl)-3(R)-[3-(4-fluorophenyl)-3(S)-hydroxypropyl]-4(S)-(4-hydroxyphenyl)-2-azetidinone. The empirical formula is C₂₄H₂₁F₂NO₃.

In one embodiment, NPC1L1 antagonists are represented by structural formula I:

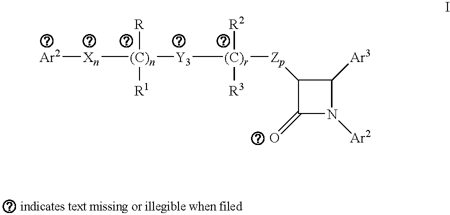

or isomers thereof, or pharmaceutically acceptable salts or solvates of the compounds of Formula (I) or of the isomers thereof, or prodrugs of the compounds of Formula (I) or of the isomers, salts or solvates thereof, wherein in Formula (I) above:
Ar¹ and Ar² are independently selected from the group consisting of aryl and R⁴-substituted aryl;
Ar³ is aryl or R⁵-substituted aryl;
X, Y and Z are independently selected from the group consisting of —CH₂—, —CH(lower alkyl)- and —C(dilower alkyl)-;
R and R² are independently selected from the group consisting of —OR⁶, —O(CO)R⁶, —O(CO)OR⁹ and —O(CO)NR⁶R⁷;
R¹ and R³ are independently selected from the group consisting of hydrogen, lower alkyl and aryl;
q is 0 or 1;
r is 0 or 1;
m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;
R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂ NR⁶R⁷, S(O)₀₋₂R⁹, —O(CH₂)_{1-10′}—COOR⁶, —O(CH₂)₁₋₁₀CONR⁶R⁷, —(lower alkylene)COOR⁶, —CH═CH—COOR⁶, —CF₃, —CN, —NO₂ and halogen;
R⁵ is 1-5 substituents independently selected from the group consisting of —OR⁶, —O(CO)R⁶, —O(CO) OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂ R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, —(lower alkylene)COOR⁶ and —CH═CH—COOR⁶;
R⁶, R⁷ and R⁸ are independently selected from the group consisting of hydrogen, lower alkyl, aryl and aryl-substituted lower alkyl; and
R⁹ is lower alkyl, aryl or aryl-substituted lower alkyl.

In another embodiment, the azetidinone or substituted β-lactam is represented by structural formula II:

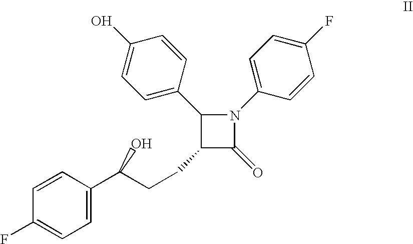

or pharmaceutically acceptable salt or solvate thereof, or prodrug of the compound of Formula (II) or of the salt or solvate thereof.

In other embodiments of the invention, the drug or compound includes any azetidinone or substituted β-lactam disclosed in U.S. Patent Application Publication No. US 2002/0151536A1, or any sugar-substituted 2-azetidinone described in U.S. Pat. No. 5,756,470.

### VIII. Additional Embodiments

In an additional embodiment, the invention provides a method for testing a subject for susceptibility for a health risk level of plasma cholesterol. The method comprises detecting the presence or absence of guanine at position 34,067 of SEQ ID NO: 1 in the subject's NPC1L1 gene and generating a test report for the subject which indicates whether guanine is present or absent in the subject. In a preferred embodiment, if guanine is present, the test report indicates that the subject is susceptible for a health risk level of plasma cholesterol. In another preferred embodiment, if guanine is absent, the test report indicates that the subject tested negative for a polymorphism associated with a health risk level of plasma cholesterol.

In another aspect, the invention provides a method of testing a human subject for the presence or absence of an NPC1L1 marker that is associated with an increased LDL-C response to an NPC1L1 antagonist. The method comprises determining the copy number in the subject's NPC1L1 gene of an allele that is associated with the response, using the determined copy number to assign to the subject the presence or absence of the NPC1L1 marker and generating a test report which indicates whether the NPC1L1 marker is present or absent in the individual. The term “determining the copy number” is meant to mean that at least one copy of the subject's NPC1L1 gene is genotyped, thus there is no requirement that both copies of a subject's NPC1L1 gene be genotyped, though typically that will be the case Thus, as shown herein, the determination of the presence of one copy of an inventive NPC1L1 marker is sufficient for the practice of the inventive methods. In one embodiment, the allele comprises adenine at position 5,400 of SEQ ID NO: 1 or guanine at position 7,096 of SEQ ID NO: 1, and if the subject's copy number for the allele is 1 or 2, the presence of the NPC1L1 marker is assigned to the subject, whereas if the subject's copy number for the allele is 0, the absence of the NPC1L1 marker is assigned to the subject. Preferably, the allele comprises adenine, adenine and guanine at positions 5,285, 5,400 and 7,096 of SEQ ID NO:1, respectively. In another embodiment, the allele comprises guanine, cytosine and cytosine at positions 5,285, 5,400 and 7,096 of SEQ ID NO:1, respectively, and if the subject's copy number for the allele is 0, the presence of the NPC1L1 marker is assigned to the subject, whereas if the subject's copy number for the allele is 1 or 2, the absence of the NPC1L1 marker is assigned to the subject. In a preferred embodiment, if the presence of the NPC1L1 marker is assigned to the subject, the test report further indicates that the subject is likely to exhibit a higher than average LDL-C response to the NPC1L1 antagonist, while if the absence of the NPC1L1 marker is assigned to the subject, the test report indicates that the subject is likely to exhibit an average LDL-C response to the NPC1L1 antagonist.

In yet another aspect, the invention provides a method of predicting the LDL-C response of a subject to an NPC1L1 antagonist. The method comprises determining the presence or absence in the subject of an NPC1L1 marker that is associated with an increased LDL-C response to an NPC1L1 antagonist, and making a prediction based on the results of the determining step. If the marker is present, the prediction is that the subject is likely to exhibit a higher than average LDL-C response to the NPC1L1 antagonist and if the marker is absent, the prediction is that the subject is likely to exhibit an average LDL-C response to the NPC1L1 antagonist.

Yet another aspect of the invention provides a method of selecting a therapy for a patient who is in need of reducing LDL-C. The method comprises determining the presence or absence in the patient of an NPC1L1 marker, and selecting the therapy based on the results of the determining step.

Another aspect of the invention is the use of an NPC1L1 antagonist in the manufacture of a medicament for lowering LDL-C in a human, wherein the medicament is designed to deliver an effective amount of the NPC1L1 antagonist to patients identified as having the NPC1L1 genetic marker.

In a still further aspect, the invention provides a method for seeking regulatory approval of a pharmacogenetic indication for a pharmaceutical formulation comprising a NPC1L1 antagonist. The method comprises demonstrating that a first group of patients having an NPC1L1 marker exhibits a mean LDL-C response to the antagonist that is higher, to a statistically significant degree, than the mean LDL-C response of a second group of patients lacking the NPC1L1 marker, and filing with a regulatory agency an application for approval to market the formulation with a label that recommends selecting the starting dose of the formulation for a patient based on whether the NPC1L1 marker is present or absent in the patient.

In a still further aspect, the invention provides a method of determining whether a genetic variant in the NPC1L1 gene is correlated with the efficacy of an NPC1L1 antagonist. In one embodiment, the method comprises obtaining an efficacy measurement for each individual in a group of individuals treated with the antagonist, identifying the genotypes for the NPC1L1 variant in each individual in the group, and performing a genetic association analysis using the efficacy measurements and the genotypes.

In another embodiment, the method comprises determining the degree of linkage disequilibrium between the genetic variant and the allele in an NPC1L1 marker, wherein a high degree of linkage disequilibrium indicates that the genetic variant is correlated with the efficacy of the antagonist and a low degree of linkage disequilibrium indicates the genetic variant is not correlated with the efficacy. In preferred embodiments, the efficacy measurement is an individual's LDL-C response to the antagonist.

A. Pharmacogenetic Treatment Methods

Pharmacogenetic treatment methods of the invention may involve determining the presence or absence in an individual of each of NPC1L1 markers 2-5 in Table 1. Pharmacogenetic treatment methods include the following specific embodiments.

A method of selecting a therapy for a human individual in need of reducing her level of plasma LDL-C, the method comprising determining the presence or absence in the individual of marker in the human Niemann pick C1-Like 1 (NPC1L1) gene that is associated with an increased LDL-C response to and NPC1L1 antagonist; and selecting the therapy based on the results of the determining step. In some embodiments, the individual has tested positive for an NPC1L1 marker that is associated with a health risk level of LDL-C.

B. Pharmacogenetic Drug Products: Manufacture and Marketing

Pharmacogenetic drug products of the invention include the following specific embodiments.
1. The use of an antagonist of Niemann pick C1-Like 1 (NPC1L1) in the manufacture of a medicament for lowering LDL-C levels in humans, wherein the medicament is formulated to deliver an effective amount of the NPC1L1 antagonist to patients who test positive for an NPC1L1 marker associated with an increased LDL-C response to the NPC1L1 antagonist.
a. In a preferred embodiment, the NPC1L1 antagonist is ezetimibe. Preferably, the NPC1L1 marker comprises: (i) 1 or 2 copies of adenine at position 5,400 of SEQ ID NO: 1; (ii) 1 or 2 copies of guanine at position 7,096 of SEQ ID NO: 1; or 1 or 2 copies of adenine, adenine and guanine at positions 5,285, 5,400 and 7,096 of SEQ ID NO: 1, respectively.
A method of marketing a drug product which comprises ezetimibe, the method comprising promoting to a target audience the use of a particular starting NPC1L1 antagonist (e.g., ezetimibe) and/or statin taking into account Niemann pick C1-Like 1 (NPC1L1) markers. Preferably, the NPC1L1 marker comprises (i) 1 or 2 copies of adenine at position 5,400 of SEQ ID NO: 1; (ii) 1 or 2 copies of guanine at position 7,096 of SEQ ID NO: 1; or (iii) 1 or 2 copies of adenine, adenine and guanine at positions 5,285, 5,400 and 7,096 of SEQ ID NO:1, respectively. In a more preferred embodiment, the promoting step further comprises providing information to the target audience on how to test patients for the NPC1L1 marker. The information preferably comprises a specific test approved by a regulatory agency.
2. A manufactured drug product, which comprises: a pharmaceutical formulation comprising an antagonist of Niemann pick C1-Like 1 (NPC1L1); and prescribing information which recommends testing a patient for the presence or absence of an NPC1L1 marker that is associated with an increased LDL-C response to the NPC1L1 antagonist and selecting the starting dose of the drug product for the patient based on whether the patient tests positive or negative for the LDL-C response marker.
a. In preferred embodiments, the NPC1L1 antagonist is ezetimibe and the NPC1L1 marker comprises (i) 1 or 2 copies of adenine at position 5,400 of SEQ ID NO: 1; (ii) 1 or 2 copies of guanine at position 7,096 of SEQ ID NO: 1; or (iii) 1 or 2 copies of adenine, adenine and guanine at positions 5,285, 5,400 and 7,096 of SEQ ID NO: 1, respectively. In one particularly preferred embodiment, the pharmaceutical formulation is a tablet comprising ezetimibe and a pharmaceutically acceptable carrier. Preferably, the tablet further comprises a pharmaceutically effective amount of a statin. A method of manufacturing a pharmacogenetic drug product, the method comprising: combining in a package a pharmaceutical formulation comprising ezetimibe and prescribing information. The prescribing information comprises instructions for testing a patient for the presence or absence of a marker in the Niemann pick C1-Like 1 (NPC1L1) gene that is associated with an increased LDL-C response to ezetimibe and selecting the starting dose of the drug product based on the patient's test results.
b. In one preferred embodiment, the NPC1L1 antagonist is ezetimibe and the NPC1L1 marker comprises (i) 1 or 2 copies of adenine at position 5,400 of SEQ ID NO: 1; (ii) 1 or 2 copies of guanine at position 7,096 of SEQ ID NO: 1; or (iii) 1 or 2 copies of adenine, adenine and guanine at positions 5,285, 5,400 and 7,096 of SEQ ID NO:1, respectively.
c. In another preferred embodiment, the pharmaceutical formulation further comprises a statin.

### Examples

Examples are provided below to further illustrate different features and advantages of the present invention. The examples also illustrate useful methodology for practicing the invention. These examples do not limit the claimed invention.

The human NPC1L1 gene maps to chromosome 7p13, spans approximately 29 Kb, and contains 20 exons (Davis, et al., (2004) J. Biol. Chem. 279: 33586-92. Several single nucleotide polymorphisms (SNPs) have been reported within NPC1L1 through the public SNP mapping effort (http://www.ncbi.nlm.nih.gov/SNP). However, the functional significance of these variants is unknown and relatively few have reported minor allele frequencies (MAFs) greater than 10%. To more fully characterize the extent of DNA sequence variation in NPC1L1 and to assess whether polymorphisms in NPC1L1 are associated with changes in selected blood component levels, the gene was re-sequenced in a large number of individuals from three different self-reporting ethnic populations, in particular to identify novel polymorphisms that may have direct functional consequences and to better estimate allele frequencies in known and novel polymorphisms. Genotyping assays were developed for a number of novel and known common variants with minor allele frequencies greater than 2%. Genetic association analysis was then performed with these polymorphisms in a clinical trial cohort to assess whether DNA sequence polymorphisms in NPC1L1 associated with changes in various plasma and blood component levels, in particular, total plasma cholesterol, low-density lipoprotein cholesterol (LDL-C), non-high-density lipoprotein cholesterol (non-HDL-C)), plasma triglyceride levels, blood Apolipoprotein A-1, or blood Apolipoprotein B (apoB) levels in response to pharmacotherapy with ezetimibe (see Example 3, Tables 4a-d).

To characterize the extent of variation in NPC1L1, all exons, conserved regulatory regions, the promoter region, and select intronic regions were resequenced in 375 normal individuals representing three ethnic groups. In total, 140 SNPs and five insertions/deletions were identified in this cohort. A complete list of these polymorphisms is described in Example 1. Of the 140 SNPs identified, 14 were located in the 5′ UTR or promoter region, 89 in introns, three in the 3′ UTR, and 34 in the coding region, with 20 of these leading to amino acid changes (see Example 1, Table 4). Table 5 (Example 2) lists the 24 SNPs that had minor allele frequencies (MAF)>4% detected in at least one ethnic group. The resequenced region of NPC1L1 spanned 20,094 bases, so that the average number of SNPs per kilo base was 0.083725 for common SNPs and 6.96725 over all SNPs, consistent with numbers reported over broader sets of genes (Crawford, et al., 2004). Using selected genotypes assays based on the above-identified SNPs, a subset of SNPs and combinations of SNPs (haplotypes) within the NPC1L1 gene were found to enhance human responsiveness to the cholesterol management drug, ezetimibe. Significant associations were observed between individual SNPs in NPC1L1 and a three NPC1L1 SNP haplotype and the degree of reduction of LDL-C after treatment with ezetimibe in the same clinical trail subjects (see Example 3, Tables 8-12).

### Example 1

### Identification of NPC1L1 Polymorphisms

To identify SNPs in NPC1L1, the promoter and coding regions of NPC1L1 were sequenced from anonymous, reportedly healthy individuals self-reporting as Caucasian (n=198), Black (n=99) or Hispanic (n=78). DNA samples were obtained from the Caucasian and African American Human Variation Panels collected by the Human Genetic Cell Repository of the National Institute for General Medical Sciences (NIGMS; Coriell Cell Repository, Camden, N.J.) as well as anonymous donors from Schering-Plough Corporation. All samples came from individuals who provided informed consent to be part of a DNA polymorphism discovery resource. Information on ethnicity and gender was collected for each individual in order to assemble the resource, but all identifying and phenotypic information has been removed from the individual samples so that links to individual donors are irreversibly broken.

Polymerase Chain Reaction

The general strategy for SNP discovery is as previously described (Nickerson et al, (1998) Nat. Genet., 19:23340) with modifications as detailed. PCR primers were designed using the Primer3 software (Rozen and Skaletsky, (2000) Methods Mol. Biol., 132:365-86; available at http://www.genome.wi.mit.edu/cgi-bin/primer/primer3.cgi) to amplify 400-650 basepair segments of the NPC1L1 coding region as well as approximately two kilobasepairs of the 5′ promoter region and 100 nucleotides flanking the intron/exon splice junctions. Forward and reverse primers used to amplify various NPC1L1 gene regions for SNP analysis were 5′ tailed with universal sequencing primers: −21M13; 5′ TGTAAAACGACGGCCAGT (SEQ D NO: 6 and M13REV; CAGGAAACAGCTATGACC (SEQ ID NO 7), respectively. Table 3 shows the NPC1L1 PCR assay primer sequences that were 5′tailed with universal sequencing primers (SEQ ID NO: 6 or SEQ ID NO: 7) and their corresponding positions relative to the genomic NPC1L1 gene sequence as set forth in SEQ ID NO: 1.

TABLE 3
NPC1L1 PCR Assay Primer Sequences

| Position | | | | | |
|---|---|---|---|---|---|
| Relative | | | | PCR | |
| to SEQ | | | | product | Anneal- |
| ID | | | Region | size | ing |
| NO: 1 | Forward Primer (5′-3′) | Reverse Primer (5′-3′) | Covered | (bp) | temp |
| 3182- | AGAATGGTAAACATTGTACTCTGAC | TTCATATGTTTCTTCCCATGGG | | 563 | 61° C. |
| 3709 | SEQ ID NO: 8 | SEQ ID NO: 9 | | | |
| 4749- | GAGCAAAGGAGAGTCTTCCACTATC | CAAGGGCTGAACACACATTAAG | 5′ | 652 | 64° C. |
| 5365 | SEQ ID NO: 10 | SEQ ID NO: 11 | promoter | | |
| 4280- | TGTCTTGAGAACTTAGGGGTCAG | CACTGTCATCCCTAGCAACTGT | 5′ | 686 | 64° C. |
| 4930 | SEQ ID NO: 12 | SEQ ID NO: 13 | promoter | | |
| 5121- | CTAATAGCGTGGTCTCTCCCCTA | ATCCCTCATGTGTCCAGAGACT | 5′UTR/ | 532 | 68° C. |
| 5617 | SEQ ID NO: 4 | SEQ ID NO: 5 | Exon 1/ | | |
| | | | Intron 1 | | |
| 6101- | GACTTTCCTAAGCTGCAGGTCTATC | GTTCACAAAATTGTCAGAGCAGG | Intron1/ | 581 | 61° C. |
| 6646 | SEQ ID NO: 14 | SEQ ID NO: 15 | Exon 2 | | |
| 6624- | CTGCTCTGACAATTTTGTGAACCT | AGACAGAGCAGAGGATGATGATG | Exon 2 | 575 | 66° C. |
| 7163 | SEQ ID NO: 16 | SEQ ID NO: 17 | | | |
| 6404- | ACCCAGAGCTGTCTGGAAGCCTCATG | CCATTGCCTGTGTCTCCCTGGA | Exon 2 | 547 | 64° C. |
| 6915 | SEQ ID NO: 18 | SEQ ID NO: 19 | | | |
| 7093- | CTCGACTCCACCTTCTACCTGG | CAGAGAGTCATACCTGTAGCTGGAC | Exon 2 | 498 | 64° C. |
| 7555 | SEQ ID NO: 20 | SEQ ID NO: 21 | | | |
| 7460- | AAGCTTTCCATGACCAGCATTT | AGCCGTAGGAATAGCTACCTCTG | Exon 2/ | 562 | 66° C. |
| 7986 | SEQ ID NO: 22 | SEQ ID NO: 23 | Intron 2 | | |
| 8546- | AGTACTCCATACTCCAGAGCAAATG | GTATTGAGGTTAGATTTGGAACCCT | Intron 2 | 721 | 63° C. |
| 9231 | SEQ ID NO: 24 | SEQ ID NO: 25 | | | |
| 8160- | TCTTGCTTTAAGTCTGACAGAGGAG | GTTCCTGCTATTTCCAAGAGAGAG | Intron 2 | 702 | 68° C. |
| 8826 | SEQ ID NO: 26 | SEQ ID NO: 27 | | | |
| 9554- | CGTCCTAAATAGCTAAATGGCCTAA | CCACAGTGCCTGAGTAACACTACTA | Intron 2/ | 517 | 64 C. |
| 10035 | SEQ ID NO: 28 | SEQ ID NO: 29 | Exon 3/ | | |
| | | | Intron 3 | | |
| 8974- | TTTACAGACAGGAAAACTGAGGTTC | CTGCATTTAGGCCATTTAGCTATT | Intron 2 | 647 | 57° C. |
| 9585 | SEQ ID NO: 30 | SEQ ID NO: 31 | | | |
| 10072- | AGAGAAGTGGGGTGTAGGAGGTAAG | TATAATCGCAGGTGAGGCTATAAGA | Intron 3/ | 554 | 66° C. |
| 10590 | SEQ ID NO: 32 | SEQ ID NO: 33 | Exon 4/ | | |
| | | | Intron 4 | | |
| 10465- | GTCTTGGGTCAGTTCCTGTGTC | AGAGGTATTACCCTTTGGGGCA | Intron 4/ | 553 | 68° C. |
| 10982 | SEQ ID NO: 34 | SEQ ID NO: 35 | Exon 5/ | | |
| | | | Intron 5 | | |
| 11060- | CTTTTCTCTTCTCTTTTCCCTCCTA | GCTCACACCTGTAATCTCAACATTT | Intron 5 | 687 | 63° C. |
| 11711 | SEQ ID NO: 36 | SEQ ID NO: 37 | | | |
| 11806- | ATGCTCAAGGAAGATGGAGTAGG | GTGTCGATGAACAGAAAGAGTCTG | Intron 5/ | 586 | 64° C |
| 12356 | SEQ ID NO: 38 | SEQ ID NO: 39 | Exon 6/ | | |
| | | | Intron 6 | | |
| 12685- | AGTCTCTGATGATTCAGGAAGGTC | AATATTACTCTCCTGGCACAATGC | Intron 6/ | 736 | 64° C. |
| 13385 | SEQ ID NO: 40 | SEQ ID NO: 41 | Exon 7/ | | |
| | | | Intron 7/ | | |
| | | | Exon 8/ | | |
| | | | Intron 8 | | |
| 12519- | CATTCCATGGTAAGGATAAATCAGA | ACATCTGCAGGAGGAAGTCAAG | Intron 6/ | 719 | 66° C. |
| 13202 | SEQ ID NO: 42 | SEQ ID NO: 43 | Exon 7/ | | |
| | | | Intron 7/ | | |
| | | | Exon 8 | | |
| 12519- | CATTCCATGGTAAGGATAAATCAGA | AATATTACTCTCCTGGCACAATGC | Intron 6/ | 902 | 64° C. |
| 13385 | SEQ ID NO: 44 | SEQ ID NO: 45 | Exon 7/ | | |
| | | | Intron 7/ | | |
| | | | Exon 8/ | | |
| | | | Intron 8 | | |
| 13532- | TAAGCAGTTGAAAATCTGCATGTAA | CTCTTCCTCAGCCTACTCAACCT | Intron 8 | 622 | 68° C. |
| 14118 | SEQ ID NO: 46 | SEQ ID NO: 47 | | | |
| 13173- | AGTGATCCTTGACTTCCTCCTG | TGAAACCCCATCTCTATTAAAAACA | Exon 8/ | 616 | 64° C. |
| 13753 | SEQ ID NO: 48 | SEQ ID NO: 49 | Intron 8 | | |
| 14719- | AAGTCTGCTCAACTCCAGAATGTT | CTGTTGTGCTGTTCATACACGAAT | Intron 9/ | 428 | 68° C. |
| 15111 | SEQ ID NO: 50 | SEQ ID NO: 51 | Exon 10/ | | |
| | | | Intron 10 | | |
| 14228- | TATAAATGAGAGGTCGACAGGAGTT | ACAAATTTAAGTCAGTCAGGGTGTC | Intron 8/ | 582 | 68° C. |
| 14774 | SEQ ID NO: 52 | SEQ ID NO: 53 | Exon 9/ | | |
| | | | Intron 9 | | |
| 14165- | GAAGAGAATCCAGGGATAAGTGAG | AAATTTAAGTCAGTCAGGGTGTCAT | Intron 8/ | 643 | 64° C. |
| 14772 | SEQ ID NO: 54 | SEQ ID NO: 55 | Exon 9/ | | |
| | | | Intron 9 | | |
| 15582- | CACAGACAACAAAGTCTGAGACACA | AAATGTCCCCAACAGAAAAATAAAC | Intron 10 | 523 | 64° C. |
| 16069 | SEQ ID NO: 56 | SEQ ID NO: 57 | | | |
| 15025- | AGAGGTGCAGAATTGTTCATTACTC | ATGTGTCTCAGACTTTGTTGTCTGT | Intron 10 | 619 | 64° C. |
| 15608 | SEQ ID NO: 58 | SEQ ID NO: 59 | | | |
| 16254- | AACTTTACCCAACAAACAGTGACTC | GCGAAACCCTGTCTCTACTAAAAGT | Intron 10 | 606 | 65° C. |
| 16824 | SEQ ID NO: 60 | SEQ ID NO: 61 | | | |
| 15857- | ACTGTACTTTGGGTGACTTTATGGA | GAGTCACTGTTTGTTGGGTAAAGTT | Intron 10 | 458 | 65° C. |
| 16279 | SEQ ID NO: 62 | SEQ ID NO: 63 | | | |
| 16936- | TTCTATGAGTTTGACCACTCTAGGC | ATTAAACACACACACACACACACAC | Intron 10 | 671 | 64° C. |
| 17571 | SEQ ID NO: 64 | SEQ ID NO: 65 | | | |
| 17363- | TTTTTCTGTTCTTCCACTTTCAATC | AAAAGAGAGTAGTAGGACCAGGCAT | Intron 10 | 578 | 64° C. |
| 17905 | SEQ ID NO: 66 | SEQ ID NO: 67 | | | |
| 18964- | TACCTTTGCCAGGGATTTATTTATT | TGAAGGAATTCGTTATCACTAGACC | Intron 10 | 655 | 64° C. |
| 19583 | SEQ ID NO: 68 | SEQ ID NO: 69 | | | |
| 21043- | CTTGAGTAGCTGGGACTACAGGTAT | ATTCAAAAGCAGTCAGAAGAAAGAA | Intron 10 | 729 | 63° C. |
| 21736 | SEQ ID NO: 70 | SEQ ID NO: 71 | | | |
| 21810- | TCCTCATTGATATTTCCATTTTGTT | AAAAATGCAGTCTCAAAAATACCTG | Intron 10 | 725 | 63° C. |
| 22499 | SEQ ID NO: 72 | SEQ ID NO: 73 | | | |
| 22449- | CAAAGGCACAGAGTTAATGTCTTCT | ACACTTGTAATTTCAGAACTTTGGG | Intron 10 | 662 | 63° C. |
| 23075 | SEQ ID NO: 74 | SEQ ID NO: 75 | | | |
| 24700- | CTGATGTTCTATCCCTGTCCTG | CACCTACAAATGCCACTGCTTT | Intron 11/ | 647 | 65° C. |
| 25311 | SEQ ID NO: 76 | SEQ ID NO: 77 | Exon 12/ | | |
| | | | Intron 12 | | |
| 24177- | TGCATGTACCTCTGTGTACCTCTAA | ACAGGGATAGAACATCAGGAAGAG | Intron 10/ | 577 | 68° C. |
| 24718 | SEQ ID NO: 78 | SEQ ID NO: 79 | Exon 11/ | | |
| | | | Intron 11 | | |
| 25375- | CCACAGTTTCTATAGCCAAGAGGA | AGTCAAGTTCACAGAGGTGCTGTAT | Intron 12/ | 554 | 68° C. |
| 25893 | SEQ ID NO: 80 | SEQ ID NO: 81 | Exon 13/ | | |
| | | | Intron 13/ | | |
| | | | Exon 14 | | |
| 25620- | GAGCAGTTCCATAAGTATCTTCCCT | GAATCAATTCCACAAACTTAGCACT | Exon 13/ | 554 | 68° C. |
| 26138 | SEQ ID NO: 82 | SEQ ID NO: 83 | Intron 13/ | | |
| | | | Exon 14/ | | |
| | | | Intron 14 | | |
| 28070- | ACCTCTACCTCCTGGATTCAAGTAA | ATCTTGGCTCACTGCAACTTCT | Intron 14 | 409 | 64° C. |
| 28443 | SEQ ID NO: 84 | SEQ ID NO: 85 | | | |
| 28070- | ACCTCTACCTCCTGGATTCAAGTAA | CTTGTTTTTGTTTTCGAGACAGAGT | Intron 14 | 468 | 65° C. |
| 28502 | SEQ ID NO: 86 | SEQ ID NO: 87 | | | |
| 29174- | TACTAAGAATTTCAAATGGTGGTGG | GGTACAAACCAGCCTAAGAAATAGG | Intron 14/ | 494 | 68° C. |
| 29632 | SEQ ID NO: 88 | SEQ ID NO: 89 | Exon 15/ | | |
| | | | Intron 15 | | |
| 29511- | GTTGCTGGAGACTGGAGGTTAG | AACTAGGAGTATTCTATGAGGCTGG | Intron 15/ | 576 | 68° C. |
| 30051 | SEQ ID NO: 90 | SEQ ID NO: 91 | Exon 16/ | | |
| | | | Intron 16 | | |
| 30315- | AAAGTGTTGGGATTATAGGCATGAG | AAGAAGAAGATCTGAATGAGCTGG | Intron 16/ | 518 | 68° C. |
| 30797 | SEQ ID NO: 92 | SEQ ID NO: 93 | Exon 17/ | | |
| | | | Intron 17/ | | |
| | | | Exon 18 | | |
| 29935- | ATCAGTTACAATGCTGTGTCCCTC | GGGAAGGAACTAGGGAGATGAG | Exon 16/ | 538 | 68° C. |
| 30437 | SEQ ID NO: 94 | SEQ ID NO: 95 | Intron 16 | | |
| 30494- | GTGGAGTTTGTGTCCCACATTA | ATAGTAGCTTCCAAGACAGAATTGC | Exon 17/ | 579 | 68° C. |
| 31037 | SEQ ID NO: 96 | SEQ ID NO: 97 | Intron 17/ | | |
| | | | Exon 18 | | |
| 33277- | TATGGGGATCTTCCTTGTGACTG | CTTATGAGAGCATCCTTCCTGG | Exon 19/ | 586 | 68° C. |
| 33827 | SEQ ID NO: 98 | SEQ ID NO: 99 | 3′ UTR | | |
| 32874- | CTTGGGCTGTGAACATAGTGAC | CTCCAGTGACAGGCAGTCTCAT | Intron 18/ | 725 | 68° C. |
| 33367 | SEQ ID NO: 100 | SEQ ID NO: 101 | Exon 19 | | |
| 33611- | AAGTCTTTAACACGTAGCAGTGTCC | AAAGAGGGAGGAGAAATAGAACAAA | Exon 19/ | 710 | 66° C. |
| 34285 | SEQ ID NO: 102 | SEQ ID NO: 103 | 3′ UTR | | |

PCR reactions contained genomic DNA (24 ng) in the presence of Platinum PCR Supermix High Fidelity (100 μM dNTPs, 1.5 mM MgCl₂, 0.1 U Platinum Taq polymerase High Fidelity, Invitrogen Corp., Carlsbad, Calif.) and 0.2 pmol/μl forward and reverse primers in 12 μl total volume. Thermocycling was performed in 96-well microplates (PTC-200 thermocycler, MJ Research) with an initial denaturation at 94° C. for 5 minutes (min) followed by 35 cycles of denaturation at 94° C. for 30 seconds (s), primer annealing (see Table 3 for primer specific temperatures) for 30 s, and primer extension at 68° C. for 1 min. After 35 cycles, a final extension was carried out for 7 minutes at 68° C.

DNA Sequencing and Analysis

Following DNA amplification, PCR reactions were diluted to 50 μl in PCR buffer containing 0.5 μl of ExoSAP-IT (USB Corporation, Cleveland, Ohio) and were incubated 15 min at 37° C. followed by inactivation of the enzymes at 80° C. for 15 min. Cycle sequencing in the forward and reverse directions was performed using ABI PRISM BigDye terminator v3.1 Cycle Sequencing DNA Sequencing Kit (Applied Biosystems, Foster City, Calif.) according to manufacture's instructions. Briefly, 1 μl of each PCR product was used as template and combined with 4 μl sequencing reaction mix containing 5 pmol M13 sequencing primer (−21M13 or M13Rev), 0.5× Sequencing buffer and 0.25 μl BDTv3.1 mix. Sequencing reactions were denatured for 1 min at 96° C. followed by 25 cycles at 96° C. for 10 s, 50° C. for 5 s and 60° C. for 4 min. Sequencing reactions were purified by filtration using Montage SEQ384 plates (Millipore Corp. Bedford, Mass.), dissolved in 25 μl deionized water and resolved by capillary gel electrophoresis on an Applied Biosystems 3730XL DNA Analyzer. Chromatograms were transferred to a Unix workstation (DEC alpha, Compaq Corp), base called was performed with Phred software (version 0.990722.g), sequences were assembled with Plrap software (version 3.01)(Nickerson, et al., (1997) Nucleic Acid Res., 25:2745-51), scanned with Polyphred software (version 3.5) (Nickerson, et al., (1997) Nucleic Acid Res., 25:2745-51), and the results were viewed with Consed software (version 9.0) (Gordon et al., (1998) Genome Res., 8:195-202). Analysis parameters were all maintained at the individual software's default settings. The Phred, Phrap and Consed software programs are available at http://www.genome.washington.edu, and the PolyPhred software program is available at http://droog.mbt.washington.edu).

SNP Analysis Results

The human NPC1L1 gene maps to chromosome 7p13 and contains 20 exons spanning approximately 29 Kb of genomic DNA. Several single nucleotide polymorphisms (SNPs) have been reported within NPC1L1 through the public SNP mapping effort (http://www.ncbi.nlm.nih.gov/SNP). However, the functional significance of these variants is unknown and relatively few have reported minor allele frequencies (MAFs) greater than 10%. To characterize the extent of variation in NPC1L1, all exons, conserved regulatory regions, the promoter region, and select intronic regions were resequenced in 375 normal individuals representing three ethnic groups (the resequencing cohort). In total, 140 SNPs and five insertions/deletions were identified in this cohort. SNP names were assigned according to the convention proposed by den Dunnen and Anonarakis ((2000), Hum. Mutat. 15:7-12). A complete list of the 140 NPC1L1 polymorphisms is given in Table 4.

TABLE 4
NPC1L1 Polymorphisms and Allele Frequency Analysis
in African American, Caucasian and Hispanic Cohorts

| | Position | | | | | | |
|---|---|---|---|---|---|---|---|
| | Relative | | | | | | |
| | to ATG | | | | | Allele | |
| | on | Position | | | | Frequency | |
| Position | Genomic | Relative to | | | | Analysis | |
| Relative | DNA | ATG on | | | | African | |
| to SEQ | [SEQ ID | cDNA | Major | Minor | AA | American | |
| ID NO: 1 | NO: 1] | NM_013389 | Allele | Allele | Change | Major | Minor |
| 1151 | −4267 | | G | A | — | 0 | 0 |
| 1224 | −4194 | | A | G | — | 0 | 0 |
| 1250 | −4168 | | T | C | — | 0 | 0 |
| 2961 | −2457 | | G | A | — | 0 | 0 |
| 3311 | −2107 | | A | G | — | 89 | 1 (1.11%) |
| | | | | | | (98.88%) | |
| 3396 | −2022 | | G | A | — | 89 | 1 (1.11%) |
| | | | | | | (98.88%) | |
| 3620 | −1798 | | C | T | — | 69 | 1 (1.42%) |
| | | | | | | (98.57%) | |
| 3945 | −1473 | | T | C | — | 0 | 0 |
| 4436 | −982 | | G | C | — | 188 | 10 |
| | | | | | | (94.94%) | (5.05%) |
| 4656 | −762 | | T | C | — | 190 | 6 (3.06%) |
| | | | | | | (96.93%) | |
| 4723 | −695 | | G | A | — | 189 | 5 (2.57%) |
| | | | | | | (97.42%) | |
| 5035 | −383 | | T | C | — | 191 | 5 (2.55%) |
| | | | | | | (97.44%) | |
| 5126 | −292 | | T | C | — | 194 | 2 (1.02%) |
| | | | | | | (98.97%) | |
| 5285 | −133 | | A | G | — | 179 | 19 |
| | | | | | | (90.4%) | (9.59%) |
| 5344 | −74 | | C | T | — | 186 | 2 (1.06%) |
| | | | | | | (98.93%) | |
| 5395 | −23 | | G | A | — | 187 | 1 (0.53%) |
| | | | | | | (99.46%) | |
| 5400 | −18 | | C | A | — | 176 | 8 (4.34%) |
| | | | | | | (95.65%) | |
| 5414 | −4 | | T | C | — | 188 | 0 |
| | | | | | | (100%) | |
| 5585 | 168 | | C | A | — | 175 | 5 (2.77%) |
| | | | | | | (97.22%) | |
| 6442 | 1025 | 162 | C | T | N54N | 91 | 1 (1.08%) |
| | | | | | | (98.91%) | |
| 6462 | 1045 | 182 | C | T | T61M | 91 | 1 (1.08%) |
| | | | | | | (98.91%) | |
| 6801 | 1384 | 521 | G | A | R174H | 194 | 0 |
| | | | | | | (100%) | |
| 6808 | 1391 | 528 | C | T | R176R | 194 | 0 |
| | | | | | | (100%) | |
| 6809 | 1392 | 529 | G | A | V177I | 194 | 0 |
| | | | | | | (100%) | |
| 6850 | 1433 | 570 | C | T | G190G | 188 | 0 |
| | | | | | | (100%) | |
| 6941 | 1524 | 661 | C | T | H221Y | 190 | 0 |
| | | | | | | (100%) | |
| 7027 | 1610 | 747 | C | A | D249E | 181 | 1 (0.54%) |
| | | | | | | (99.45%) | |
| 7096 | 1679 | 816 | C | G | L272L | 153 | 35 |
| | | | | | | (81.38%) | (18.61%) |
| 7097 | 1680 | 817 | G | T | D273Y | 174 | 0 |
| | | | | | | (100%) | |
| 7208 | 1791 | 928 | G | T | A310S | 191 | 9 (4.5%) |
| | | | | | | (95.5%) | |
| 7324 | 1907 | 1044 | G | A | V348V | 146 | 0 |
| | | | | | | (100%) | |
| 7358 | 1941 | 1078 | G | A | V360I | 199 | 1 (0.5%) |
| | | | | | | (99.5%) | |
| 7440 | 2023 | 1160 | A | G | N387S | 189 | 9 (4.54%) |
| | | | | | | (95.45%) | |
| 7486 | 2069 | 1206 | C | T | G402G | 155 | 1 (0.64%) |
| | | | | | | (99.35%) | |
| 7529 | 2112 | 1249 | C | T | R417W | 177 | 1 (0.56%) |
| | | | | | | (99.43%) | |
| 7776 | 2359 | 1496 | C | T | T499M | 200 | 0 |
| | | | | | | (100%) | |
| 7810 | 2393 | 1530 | G | A | M510I | 0 | 0 |
| 7870 | 2453 | | T | C | — | 200 | 0 |
| | | | | | | (100%) | |
| 7890 | 2473 | | G | A | — | 198 (99%) | 2 (1%) |
| 8475 | 3058 | | G | A | — | 194 | 0 |
| | | | | | | (100%) | |
| 8553 | 3136 | | C | A | — | 193 | 1 (0.51%) |
| | | | | | | (99.48%) | |
| 8560 | 3143 | | C | A | — | 187 | 7 (3.6%) |
| | | | | | | (96.39%) | |
| 8629 | 3212 | | C | A | — | 193 | 1 (0.51%) |
| | | | | | | (99.48%) | |
| 8654 | 3237 | | C | T | — | 174 | 16 |
| | | | | | | (91.57%) | (8.42%) |
| 8707 | 3290 | | C | T | — | 189 | 1 (0.52%) |
| | | | | | | (99.47%) | |
| 8722 | 3305 | | C | T | — | 194 | 0 |
| | | | | | | (100%) | |
| 8954 | 3537 | | G | A | — | 88 (100%) | 0 |
| 10545 | 5128 | | C | T | — | 87 | 1 (1.13%) |
| | | | | | | (98.86%) | |
| 10610 | 5193 | | C | A | — | 0 | 0 |
| 10733 | 5316 | 1879 | C | T | R627C | 90 (100%) | 0 |
| 10794 | 5377 | 1940 | T | A | I647N | 89 | 1 (1.11%) |
| | | | | | | (98.88%) | |
| 11240 | 5823 | | C | T | — | 183 | 15 |
| | | | | | | (92.42%) | (7.57%) |
| 11353 | 5936 | | C | T | — | 178 | 18 |
| | | | | | | (90.81%) | (9.18%) |
| 11358 | 5941 | | C | T | — | 195 | 1 (0.51%) |
| | | | | | | (99.48%) | |
| 11386 | 5969 | | C | G | — | 195 | 1 (0.51%) |
| | | | | | | (99.48%) | |
| 11402 | 5985 | | G | C | — | 167 | 27 |
| | | | | | | (86.08%) | (13.91%) |
| 11536 | 6119 | | C | T | — | 193 | 3 (1.53%) |
| | | | | | | (98.46%) | |
| 11537 | 6120 | | G | T | — | 195 | 1 (0.51%) |
| | | | | | | (99.48%) | |
| 11538 | 6121 | | C | T | — | 195 | 1 (0.51%) |
| | | | | | | (99.48%) | |
| 11599 | 6182 | | G | T | — | 166 | 30 |
| | | | | | | (84.69%) | (15.3%) |
| 11624 | 6207 | | G | A | — | 178 | 12 |
| | | | | | | (93.68%) | (6.31%) |
| 11962 | 6545 | | G | A | — | 201 | 1 (0.49%) |
| | | | | | | (99.5%) | |
| 12087 | 6670 | 2023 | G | A | V675M | 195 | 1 (0.51%) |
| | | | | | | (99.48%) | |
| 12310 | 6893 | | C | T | — | 177 | 21 |
| | | | | | | (89.39%) | (10.6%) |
| 12864 | 7447 | 2207 | T | C | I736T | 197 | 1 (0.5%) |
| | | | | | | (99.49%) | |
| 12966 | 7549 | | G | A | — | 194 | 2 (1.02%) |
| | | | | | | (98.97%) | |
| 13162 | 7745 | 2325 | C | T | T775T | 198 | 0 |
| | | | | | | (100%) | |
| 13369 | 7952 | | G | T | — | 87 | 1 (1.13%) |
| | | | | | | (98.86%) | |
| 13577 | 8160 | | G | A | — | 91 | 1 (1.08%) |
| | | | | | | (98.91%) | |
| 13713 | 8296 | | G | A | — | 76 (100%) | 0 |
| 13718 | 8301 | | G | A | — | 71 | 5 (6.57%) |
| | | | | | | (93.42%) | |
| 13776 | 8359 | | G | A | — | 77 | 1 (1.28%) |
| | | | | | | (98.71%) | |
| 14414 | 8997 | | C | T | — | 193 | 3 (1.53%) |
| | | | | | | (98.46%) | |
| 14513 | 9096 | 2463 | T | G | P821P | 192 (96%) | 8 (4%) |
| 14555 | 9138 | 2505 | T | C | A835A | 195 | 5 (2.5%) |
| | | | | | | (97.5%) | |
| 14619 | 9202 | | C | T | — | 189 | 11 (5.5%) |
| | | | | | | (94.5%) | |
| 14648 | 9231 | | G | A | — | 188 (94%) | 12 (6%) |
| 14816 | 9399 | | A | C | — | 0 | 0 |
| 15292 | 9875 | | C | T | — | 77 | 15 |
| | | | | | | (83.69%) | (16.3%) |
| 15559 | 10142 | | G | T | — | 90 | 2 (2.17%) |
| | | | | | | (97.82%) | |
| 15583 | 10166 | | C | T | — | 84 | 6 (6.66%) |
| | | | | | | (93.33%) | |
| 15835 | 10418 | | G | A | — | 64 | 4 (5.88%) |
| | | | | | | (94.11%) | |
| 16098 | 10681 | | A | T | — | 92 (100%) | 0 |
| 16209 | 10792 | | A | G | — | 87 | 5 (5.43%) |
| | | | | | | (94.56%) | |
| 16253 | 10836 | | G | A | — | 91 | 1 (1.08%) |
| | | | | | | (98.91%) | |
| 16407 | 10990 | | C | T | — | 87 | 5 (5.43%) |
| | | | | | | (94.56%) | |
| 16535 | 11118 | | T | A | — | 78 | 14 |
| | | | | | | (84.78%) | (15.21%) |
| 16538 | 11121 | | T | G | — | 86 | 6 (6.52%) |
| | | | | | | (93.47%) | |
| 16742 | 11325 | | C | A | — | 85 | 5 (5.55%) |
| | | | | | | (94.44%) | |
| 17199 | 11782 | | T | G | — | 75 | 9 |
| | | | | | | (89.28%) | (10.71%) |
| 17199 | 11782 | | T | G | — | 75 | 9 |
| | | | | | | (89.28%) | (10.71%) |
| 17513 | 12096 | | G | A | — | 76 (100%) | 0 |
| 17524 | 12107 | | T | C | — | 72 (100%) | 0 |
| 19098 | 13681 | | G | C | — | 89 | 1 (1.11%) |
| | | | | | | (98.88%) | |
| 19359 | 13942 | | G | A | — | 90 | 2 (2.17%) |
| | | | | | | (97.82%) | |
| 19415 | 13998 | | G | A | — | 92 (100%) | 0 |
| 19426 | 14009 | | C | A | — | 92 (100%) | 0 |
| 21114 | 15697 | | T | C | — | 144 | 8 (5.26%) |
| | | | | | | (94.73%) | |
| 21200 | 15783 | | A | G | — | 74 (100%) | 0 |
| 21200 | 15783 | | A | G | — | 74 (100%) | 0 |
| 21541 | 16124 | | A | G | — | 172 | 20 |
| | | | | | | (89.58%) | (10.41%) |
| 21541 | 16124 | | A | G | — | 172 | 20 |
| | | | | | | (89.58%) | (10.41%) |
| 22118 | 16701 | | T | C | — | 0 | 0 |
| 22164 | 16747 | | T | C | — | 84 | 6 (6.66%) |
| | | | | | | (93.33%) | |
| 22203 | 16786 | | C | A | — | 89 | 1 (1.11%) |
| | | | | | | (98.88%) | |
| 22319 | 16902 | | G | A | — | 90 | 2 (2.17%) |
| | | | | | | (97.82%) | |
| 22639 | 17222 | | C | T | — | 192 | 0 |
| | | | | | | (100%) | |
| 22692 | 17275 | | G | A | — | 178 | 14 |
| | | | | | | (92.7%) | (7.29%) |
| 22708 | 17291 | | T | A | — | 155 | 37 |
| | | | | | | (80.72%) | (19.27%) |
| 22721 | 17304 | | G | A | — | 173 | 19 |
| | | | | | | (90.1%) | (9.89%) |
| 22721 | 17304 | | G | C | — | 156 | 0 |
| | | | | | | (100%) | |
| 22721 | 17304 | | A | C | — | 2 (100%) | 0 |
| 22794 | 17377 | | T | C | — | 191 | 1 (0.52%) |
| | | | | | | (99.47%) | |
| 22923 | 17506 | | G | T | — | 191 | 1 (0.52%) |
| | | | | | | (99.47%) | |
| 22992 | 17575 | | C | T | — | 141 | 45 |
| | | | | | | (75.8%) | (24.19%) |
| 24310 | 18893 | | T | C | — | 193 | 3 (1.53%) |
| | | | | | | (98.46%) | |
| 24375 | 18958 | | T | G | — | 164 | 34 |
| | | | | | | (82.82%) | (17.17%) |
| 24392 | 18975 | | G | A | — | 189 | 9 (4.54%) |
| | | | | | | (95.45%) | |
| 24641 | 19224 | | G | A | — | 179 | 7 (3.76%) |
| | | | | | | (96.23%) | |
| 24676 | 19259 | | T | C | — | 157 | 29 |
| | | | | | | (84.4%) | (15.59%) |
| 24818 | 19401 | | C | T | — | 198 | 0 |
| | | | | | | (100%) | |
| 24932 | 19515 | 2920 | C | T | P974S | 194 (97%) | 6 (3%) |
| 24961 | 19544 | 2949 | C | T | T983T | 198 | 0 |
| | | | | | | (100%) | |
| 25065 | 19648 | | G | A | — | 195 | 3 (1.51%) |
| | | | | | | (98.48%) | |
| 25694 | 20277 | | G | A | — | 189 | 3 (1.56%) |
| | | | | | | (98.43%) | |
| 25902 | 20485 | 3126 | C | G | G1042G | 182 | 0 |
| | | | | | | (100%) | |
| 28126 | 22709 | | G | A | — | 88 | 4 (4.34%) |
| | | | | | | (95.65%) | |
| 28264 | 22847 | | C | T | — | 84 | 4 (4.54%) |
| | | | | | | (95.45%) | |
| 28323 | 22906 | | C | T | — | 88 (100%) | 0 |
| 28346 | 22929 | | C | T | — | 88 (100%) | 0 |
| 28364 | 22947 | | C | T | — | 86 | 6 (6.52%) |
| | | | | | | (93.47%) | |
| 29241 | 23824 | | C | T | — | 183 | 7 (3.68%) |
| | | | | | | (96.31%) | |
| 29242 | 23825 | | G | A | — | 184 | 8 (4.16%) |
| | | | | | | (95.83%) | |
| 29383 | 23966 | 3200 | G | A | R1067Q | 199 | 1 (0.5%) |
| | | | | | | (99.5%) | |
| 29598 | 24181 | | C | T | — | 191 | 5 (2.55%) |
| | | | | | | (97.44%) | |
| 30114 | 24697 | | C | T | — | 87 | 1 (1.13%) |
| | | | | | | (98.86%) | |
| 30651 | 25234 | | A | G | — | 194 | 4 (2.02%) |
| | | | | | | (97.97%) | |
| 30703 | 25286 | | A | C | — | 137 | 57 |
| | | | | | | (70.61%) | (29.38%) |
| 30750 | 25333 | 3672 | C | T | I1224I | 191 | 1 (0.52%) |
| | | | | | | (99.47%) | |
| 30852 | 25435 | 3774 | G | A | L1258L | 190 | 4 (2.06%) |
| | | | | | | (97.93%) | |
| 30870 | 25453 | 3792 | C | T | Y1264Y | 176 | 14 |
| | | | | | | (92.63%) | (7.36%) |
| 33038 | 27621 | 3807 | T | C | V1269V | 159 | 27 |
| | | | | | | (85.48%) | (14.51%) |
| 33186 | 27769 | 3955 | G | T | G1319C | 190 | 0 |
| | | | | | | (100%) | |
| 33220 | 27803 | 3989 | G | A | R1330Q | 189 | 1 (0.52%) |
| | | | | | | (99.47%) | |
| 33463 | 28046 | | G | A | — | 175 | 1 (0.56%) |
| | | | | | | (99.43%) | |
| 33734 | 28317 | | G | T | — | 195 | 1 (0.51%) |
| | | | | | | (99.48%) | |
| 33761 | 28344 | | A | G | — | 194 | 0 |
| | | | | | | (100%) | |
| 34067 | 28650 | | A | G | — | 188 (94%) | 12 (6%) |
| 11972 | 6555 | 3118 | | | | 188 | 6 (3.09%) |
| | | | | | | (96.9%) | |
| 16643 | 11226 | 4358 | | | | 88 | 2 (2.22%) |
| | | | | | | (97.77%) | |
| 30671 | 25254 | 3711 | | | | 126 | 0 |
| | | | | | | (100%) | |
| 32977 | 27560 | 5899 | | | | 185 | 1 (0.53%) |
| | | | | | | (99.46%) | |
| 34180 | 28763 | 4949 | | | | 165 | 29 |
| | | | | | | (85.05%) | (14.94%) |

| Position | | | | | | |
|---|---|---|---|---|---|---|
| Relative | Allele Frequency Analysis | | | | | |
| to SEQ | Caucasian | | Hispanic | | Total | |
| ID NO: 1 | Major | Minor | Major | Minor | Major | Minor |
| 1151 | 0 | 0 | 0 | 0 | 0 | 0 |
| 1224 | 0 | 0 | 0 | 0 | 0 | 0 |
| 1250 | 0 | 0 | 0 | 0 | 0 | 0 |
| 2961 | 0 | 0 | 0 | 0 | 0 | 0 |
| 3311 | 92 (100%) | 0 | 0 | 0 | 181 | 1 (0.54%) |
| | | | | | (99.45%) | |
| 3396 | 92 (100%) | 0 | 0 | 0 | 181 | 1 (0.54%) |
| | | | | | (99.45%) | |
| 3620 | 68 (100%) | 0 | 0 | 0 | 137 | 1 (0.72%) |
| | | | | | (99.27%) | |
| 3945 | 0 | 0 | 0 | 0 | 0 | 0 |
| 4436 | 378 | 12 | 155 | 1 (0.64%) | 721 | 23 |
| | (96.92%) | (3.07%) | (99.35%) | | (96.9%) | (3.09%) |
| 4656 | 389 | 1 (0.25%) | 143 | 13 (8.33%) | 722 | 20 |
| | (99.74%) | | (91.66%) | | (97.3%) | (2.69%) |
| 4723 | 384 | 0 | 152 | 0 | 725 | 5 (0.68%) |
| | (100%) | | (100%) | | (99.31%) | |
| 5035 | 340 | 0 | 153 | 1 (0.64%) | 684 | 6 (0.86%) |
| | (100%) | | (99.35%) | | (99.13%) | |
| 5126 | 328 | 12 | 154 | 0 | 676 | 14 |
| | (96.47%) | (3.52%) | (100%) | | (97.97%) | (2.02%) |
| 5285 | 279 | 119 | 127 | 29 | 2271 | 797 |
| | (70.1%) | (29.89%) | (81.41%) | (18.58%) | (74.02%) | (25.97%) |
| 5344 | 396 | 0 | 142 | 0 | 724 | 2 (0.27%) |
| | (100%) | | (100%) | | (99.72%) | |
| 5395 | 396 | 0 | 140 | 0 | 723 | 1 (0.13%) |
| | (100%) | | (100%) | | (99.86%) | |
| 5400 | 340 | 56 | 125 | 9 (6.71%) | 641 | 73 (10.22%) |
| | (85.85%) | (14.14%) | (93.28%) | | (89.77%) | |
| 5414 | 395 | 1 (0.25%) | 136 | 0 | 719 | 1 (0.13%) |
| | (99.74%) | | (100%) | | (99.86%) | |
| 5585 | 377 | 3 (0.78%) | 138 | 0 | 690 | 8 (1.14%) |
| | (99.21%) | | (100%) | | (98.85%) | |
| 6442 | 94 (100%) | 0 | 0 | 0 | 2593 | 1 (0.03%) |
| | | | | | (99.96%) | |
| 6462 | 94 (100%) | 0 | 0 | 0 | 2583 | 3 (0.11%) |
| | | | | | (99.88%) | |
| 6801 | 386 | 2 (0.51%) | 156 | 0 | 3159 | 3 (0.09%) |
| | (99.48%) | | (100%) | | (99.9%) | |
| 6808 | 388 | 0 | 156 | 0 | 3156 | 2 (0.06%) |
| | (100%) | | (100%) | | (99.93%) | |
| 6809 | 387 | 1 (0.25%) | 156 | 0 | 3159 | 9 (0.28%) |
| | (99.74%) | | (100%) | | (99.71%) | |
| 6850 | 378 | 0 | 155 | 1 (0.64%) | 2727 | 3 (0.1%) |
| | (100%) | | (99.35%) | | (99.89%) | |
| 6941 | 381 | 3 (0.78%) | 156 | 0 | 3143 | 15 |
| | (99.21%) | | (100%) | | (99.52%) | (0.47%) |
| 7027 | 372 | 0 | 156 | 0 | 3137 | 1 (0.03%) |
| | (100%) | | (100%) | | (99.96%) | |
| 7096 | 288 | 82 | 109 | 43 | 1559 | 553 |
| | (77.83%) | (22.16%) | (71.71%) | (28.28%) | (73.81%) | (26.18%) |
| 7097 | 340 | 0 | 140 | 12 | 3037 | 17 |
| | (100%) | | (92.1%) | (7.89%) | (99.44%) | (0.55%) |
| 7208 | 322 | 0 | 156 | 0 | 3006 | 14 |
| | (100%) | | (100%) | | (99.53%) | (0.46%) |
| 7324 | 241 | 1 (0.41%) | 156 | 0 | 2464 | 2 (0.08%) |
| | (99.58%) | | (100%) | | (99.91%) | |
| 7358 | 324 | 0 | 156 | 0 | 2543 | 3 (0.11%) |
| | (100%) | | (100%) | | (99.88%) | |
| 7440 | 315 | 1 (0.31%) | 156 | 0 | 2040 | 18 |
| | (99.68%) | | (100%) | | (99.12%) | (0.87%) |
| 7486 | 286 | 0 | 156 | 0 | 1840 | 4 (0.21%) |
| | (100%) | | (100%) | | (99.78%) | |
| 7529 | 351 | 1 (0.28%) | 152 | 0 | 2112 | 34 |
| | (99.71%) | | (100%) | | (98.41%) | (1.58%) |
| 7776 | 393 | 1 (0.25%) | 156 | 0 | 3057 | 7 (0.22%) |
| | (99.74%) | | (100%) | | (99.77%) | |
| 7810 | 0 | 0 | 0 | 0 | 0 | 0 |
| 7870 | 390 | 2 (0.51%) | 156 | 0 | 746 | 2 (0.26%) |
| | (99.48%) | | (100%) | | (99.73%) | |
| 7890 | 392 | 0 | 156 | 0 | 746 | 2 (0.26%) |
| | (100%) | | (100%) | | (99.73%) | |
| 8475 | 388 | 2 (0.51%) | 155 | 1 (0.64%) | 737 | 3 (0.4%) |
| | (99.48%) | | (99.35%) | | (99.59%) | |
| 8553 | 392 | 0 | 156 | 0 | 741 | 1 (0.13%) |
| | (100%) | | (100%) | | (99.86%) | |
| 8560 | 391 | 1 (0.25%) | 156 | 0 | 734 | 8 (1.07%) |
| | (99.74%) | | (100%) | | (98.92%) | |
| 8629 | 392 | 0 | 156 | 0 | 741 | 1 (0.13%) |
| | (100%) | | (100%) | | (99.86%) | |
| 8654 | 342 | 52 | 144 | 12 | 660 | 80 |
| | (86.8%) | (13.19%) | (92.3%) | (7.69%) | (89.18%) | (10.81%) |
| 8707 | 381 | 13 | 155 | 1 (0.64%) | 725 | 15 |
| | (96.7%) | (3.29%) | (99.35%) | | (97.97%) | (2.02%) |
| 8722 | 389 | 1 (0.25%) | 156 | 0 | 739 | 1 (0.13%) |
| | (99.74%) | | (100%) | | (99.86%) | |
| 8954 | 85 | 1 (1.16%) | 0 | 0 | 173 | 1 (0.57%) |
| | (98.83%) | | | | (99.42%) | |
| 10545 | 92 (100%) | 0 | 0 | 0 | 179 | 1 (0.55%) |
| | | | | | (99.44%) | |
| 10610 | 0 | 0 | 0 | 0 | 0 | 0 |
| 10733 | 91 | 1 (1.08%) | 0 | 0 | 181 | 1 (0.54%) |
| | (98.91%) | | | | (99.45%) | |
| 10794 | 90 (100%) | 0 | 0 | 0 | 179 | 1 (0.55%) |
| | | | | | (99.44%) | |
| 11240 | 384 | 0 | 156 | 0 | 723 | 15 |
| | (100%) | | (100%) | | (97.96%) | (2.03%) |
| 11353 | 388 | 0 | 154 | 0 | 720 | 18 |
| | (100%) | | (100%) | | (97.56%) | (2.43%) |
| 11358 | 386 | 0 | 154 | 0 | 735 | 1 (0.13%) |
| | (100%) | | (100%) | | (99.86%) | |
| 11386 | 385 | 3 (0.77%) | 153 | 1 (0.64%) | 733 | 5 (0.67%) |
| | (99.22%) | | (99.35%) | | (99.32%) | |
| 11402 | 349 | 37 | 152 | 2 (1.29%) | 668 (91%) | 66 |
| | (90.41%) | (9.58%) | (98.7%) | | | (8.99%) |
| 11536 | 388 | 0 | 154 | 0 | 735 | 3 (0.4%) |
| | (100%) | | (100%) | | (99.59%) | |
| 11537 | 388 | 0 | 154 | 0 | 737 | 1 (0.13%) |
| | (100%) | | (100%) | | (99.86%) | |
| 11538 | 388 | 0 | 154 | 0 | 737 | 1 (0.13%) |
| | (100%) | | (100%) | | (99.86%) | |
| 11599 | 388 | 0 | 151 | 3 (1.94%) | 705 | 33 |
| | (100%) | | (98.05%) | | (95.52%) | (4.47%) |
| 11624 | 388 | 0 | 150 | 0 | 716 | 12 |
| | (100%) | | (100%) | | (98.35%) | (1.64%) |
| 11962 | 329 | 1 (0.3%) | 148 | 0 | 678 | 2 (0.29%) |
| | (99.69%) | | (100%) | | (99.7%) | |
| 12087 | 328 | 0 | 150 | 0 | 673 | 1 (0.14%) |
| | (100%) | | (100%) | | (99.85%) | |
| 12310 | 326 | 0 | 148 | 0 | 651 | 21 |
| | (100%) | | (100%) | | (96.87%) | (3.12%) |
| 12864 | 396 | 0 | 156 | 0 | 749 | 1 (0.13%) |
| | (100%) | | (100%) | | (99.86%) | |
| 12966 | 392 | 0 | 156 | 0 | 742 | 2 (0.26%) |
| | (100%) | | (100%) | | (99.73%) | |
| 13162 | 395 | 1 (0.25%) | 156 | 0 | 749 | 1 (0.13%) |
| | (99.74%) | | (100%) | | (99.86%) | |
| 13369 | 94 (100%) | 0 | 0 | 0 | 181 | 1 (0.54%) |
| | | | | | (99.45%) | |
| 13577 | 90 | 4 (4.25%) | 0 | 0 | 181 | 5 (2.68%) |
| | (95.74%) | | | | (97.31%) | |
| 13713 | 89 | 1 (1.11%) | 0 | 0 | 165 | 1 (0.6%) |
| | (98.88%) | | | | (99.39%) | |
| 13718 | 65 | 25 | 0 | 0 | 136 | 30 |
| | (72.22%) | (27.77%) | | | (81.92%) | (18.07%) |
| 13776 | 90 (100%) | 0 | 0 | 0 | 167 | 1 (0.59%) |
| | | | | | (99.4%) | |
| 14414 | 398 | 0 | 154 | 0 | 745 | 3 (0.4%) |
| | (100%) | | (100%) | | (99.59%) | |
| 14513 | 398 | 0 | 155 | 1 (0.64%) | 745 | 9 (1.19%) |
| | (100%) | | (99.35%) | | (98.8%) | |
| 14555 | 398 | 0 | 156 | 0 | 749 | 5 (0.66%) |
| | (100%) | | (100%) | | (99.33%) | |
| 14619 | 339 | 59 | 142 | 14 | 670 | 84 |
| | (85.17%) | (14.82%) | (91.02%) | (8.97%) | (88.85%) | (11.14%) |
| 14648 | 380 | 18 | 150 | 6 (3.84%) | 718 | 36 |
| | (95.47%) | (4.52%) | (96.15%) | | (95.22%) | (4.77%) |
| 14816 | 0 | 0 | 0 | 0 | 0 | 0 |
| 15292 | 90 (100%) | 0 | 0 | 0 | 167 | 15 |
| | | | | | (91.75%) | (8.24%) |
| 15559 | 77 | 13 | 0 | 0 | 167 | 15 |
| | (85.55%) | (14.44%) | | | (91.75%) | (8.24%) |
| 15583 | 92 (100%) | 0 | 0 | 0 | 176 | 6 (3.29%) |
| | | | | | (96.7%) | |
| 15835 | 83 | 1 (1.19%) | 0 | 0 | 147 | 5 (3.28%) |
| | (98.8%) | | | | (96.71%) | |
| 16098 | 93 | 1 (1.06%) | 0 | 0 | 185 | 1 (0.53%) |
| | (98.93%) | | | | (99.46%) | |
| 16209 | 63 | 31 | 0 | 0 | 150 | 36 |
| | (67.02%) | (32.97%) | | | (80.64%) | (19.35%) |
| 16253 | 94 (100%) | 0 | 0 | 0 | 185 | 1 (0.53%) |
| | | | | | (99.46%) | |
| 16407 | 94 (100%) | 0 | 0 | 0 | 181 | 5 (2.68%) |
| | | | | | (97.31%) | |
| 16535 | 92 (100%) | 0 | 0 | 0 | 170 | 14 (7.6%) |
| | | | | | (92.39%) | |
| 16538 | 87 | 5 (5.43%) | 0 | 0 | 173 | 11 |
| | (94.56%) | | | | (94.02%) | (5.97%) |
| 16742 | 92 (100%) | 0 | 0 | 0 | 177 | 5 (2.74%) |
| | | | | | (97.25%) | |
| 17199 | 94 (100%) | 0 | 0 | 0 | 169 | 9 (5.05%) |
| | | | | | (94.94%) | |
| 17199 | 94 (100%) | 0 | 0 | 0 | 169 | 9 (5.05%) |
| | | | | | (94.94%) | |
| 17513 | 85 | 1 (1.16%) | 0 | 0 | 161 | 1 (0.61%) |
| | (98.83%) | | | | (99.38%) | |
| 17524 | 81 | 1 (1.21%) | 0 | 0 | 153 | 1 (0.64%) |
| | (98.78%) | | | | (99.35%) | |
| 19098 | 94 (100%) | 0 | 0 | 0 | 183 | 1 (0.54%) |
| | | | | | (99.45%) | |
| 19359 | 92 | 2 (2.12%) | 0 | 0 | 182 | 4 (2.15%) |
| | (97.87%) | | | | (97.84%) | |
| 19415 | 94 (100%) | 0 | 0 | 0 | 186 (100%) | 0 |
| 19426 | 94 (100%) | 0 | 0 | 0 | 186 (100%) | 0 |
| 21114 | 364 | 0 | 144 | 0 | 652 | 8 (1.21%) |
| | (100%) | | (100%) | | (98.78%) | |
| 21200 | 86 (100%) | 0 | 0 | 0 | 160 (100%) | 0 |
| 21200 | 86 (100%) | 0 | 0 | 0 | 160 (100%) | 0 |
| 21541 | 299 | 97 | 143 | 13 | 614 | 130 |
| | (75.5%) | (24.49%) | (91.66%) | (8.33%) | (82.52%) | (17.47%) |
| 21541 | 299 | 97 | 143 | 13 | 614 | 130 |
| | (75.5%) | (24.49%) | (91.66%) | (8.33%) | (82.52%) | (17.47%) |
| 22118 | 0 | 0 | 0 | 0 | 0 | 0 |
| 22164 | 94 (100%) | 0 | 0 | 0 | 178 | 6 (3.26%) |
| | | | | | (96.73%) | |
| 22203 | 94 (100%) | 0 | 0 | 0 | 183 | 1 (0.54%) |
| | | | | | (99.45%) | |
| 22319 | 94 (100%) | 0 | 0 | 0 | 184 | 2 (1.07%) |
| | | | | | (98.92%) | |
| 22639 | 396 | 2 (0.5%) | 146 | 0 | 734 | 2 (0.27%) |
| | (99.49%) | | (100%) | | (99.72%) | |
| 22692 | 396 | 0 | 144 | 2 (1.36%) | 718 | 16 |
| | (100%) | | (98.63%) | | (97.82%) | (2.17%) |
| 22708 | 310 | 86 | 134 | 16 | 599 | 139 |
| | (78.28%) | (21.71%) | (89.33%) | (10.66%) | (81.16%) | (18.83%) |
| 22721 | 311 | 85 | 103 | 13 | 587 | 117 |
| | (78.53%) | (21.46%) | (88.79%) | (11.2%) | (83.38%) | (16.61%) |
| 22721 | 231 | 1 (0.43%) | 108 | 16 | 495 | 17 |
| | (99.56%) | | (87.09%) | (12.9%) | (96.67%) | (3.32%) |
| 22721 | 4 (100%) | 0 | 3 (30%) | 7 (70%) | 9 (56.25%) | 7 |
| | | | | | | (43.75%) |
| 22794 | 396 | 0 | 146 | 0 | 733 | 1 (0.13%) |
| | (100%) | | (100%) | | (99.86%) | |
| 22923 | 387 | 9 (2.27%) | 144 | 2 (1.36%) | 722 | 12 |
| | (97.72%) | | (98.63%) | | (98.36%) | (1.63%) |
| 22992 | 394 | 2 (0.5%) | 142 | 2 (1.38%) | 677 | 49 |
| | (99.49%) | | (98.61%) | | (93.25%) | (6.74%) |
| 24310 | 390 | 2 (0.51%) | 156 | 0 | 739 | 5 (0.67%) |
| | (99.48%) | | (100%) | | (99.32%) | |
| 24375 | 297 | 95 | 141 | 13 | 602 | 142 |
| | (75.76%) | (24.23%) | (91.55%) | (8.44%) | (80.91%) | (19.08%) |
| 24392 | 371 | 19 | 138 | 16 | 698 | 44 |
| | (95.12%) | (4.87%) | (89.61%) | (10.38%) | (94.07%) | (5.92%) |
| 24641 | 351 (90%) | 39 (10%) | 139 | 13 | 669 | 59 (8.1%) |
| | | | (91.44%) | (8.55%) | (91.89%) | |
| 24676 | 300 | 90 | 153 | 3 (1.92%) | 610 | 122 |
| | (76.92%) | (23.07%) | (98.07%) | | (83.33%) | (16.66%) |
| 24818 | 400 | 0 | 151 | 1 (0.65%) | 749 | 1 (0.13%) |
| | (100%) | | (99.34%) | | (99.86%) | |
| 24932 | 399 | 1 (0.25%) | 154 | 0 | 747 | 7 (0.92%) |
| | (99.75%) | | (100%) | | (99.07%) | |
| 24961 | 400 | 0 | 153 | 1 (0.64%) | 751 | 1 (0.13%) |
| | (100%) | | (99.35%) | | (99.86%) | |
| 25065 | 377 | 1 (0.26%) | 155 | 1 (0.64%) | 727 | 5 (0.68%) |
| | (99.73%) | | (99.35%) | | (99.31%) | |
| 25694 | 384 | 0 | 146 | 0 | 719 | 3 (0.41%) |
| | (100%) | | (100%) | | (99.58%) | |
| 25902 | 370 | 0 | 152 | 0 | 704 (100%) | 0 |
| | (100%) | | (100%) | | | |
| 28126 | 92 (100%) | 0 | 0 | 0 | 180 | 4 (2.17%) |
| | | | | | (97.82%) | |
| 28264 | 78 | 16 | 0 | 0 | 162 | 20 |
| | (82.97%) | (17.02%) | | | (89.01%) | (10.98%) |
| 28323 | 92 (100%) | 0 | 0 | 0 | 180 (100%) | 0 |
| 28346 | 92 (100%) | 0 | 0 | 0 | 180 (100%) | 0 |
| 28364 | 90 | 4 (4.25%) | 0 | 0 | 176 | 10 |
| | (95.74%) | | | | (94.62%) | (5.37%) |
| 29241 | 387 | 7 (1.77%) | 154 | 2 (1.28%) | 724 | 16 |
| | (98.22%) | | (98.71%) | | (97.83%) | (2.16%) |
| 29242 | 395 | 1 (0.25%) | 154 | 0 | 733 | 9 (1.21%) |
| | (99.74%) | | (100%) | | (98.78%) | |
| 29383 | 392 | 2 (0.5%) | 156 | 0 | 747 | 3 (0.4%) |
| | (99.49%) | | (100%) | | (99.6%) | |
| 29598 | 392 | 0 | 156 | 0 | 739 | 5 (0.67%) |
| | (100%) | | (100%) | | (99.32%) | |
| 30114 | 86 (100%) | 0 | 0 | 0 | 173 | 1 (0.57%) |
| | | | | | (99.42%) | |
| 30651 | 394 | 0 | 156 | 0 | 744 | 4 (0.53%) |
| | (100%) | | (100%) | | (99.46%) | |
| 30703 | 283 | 89 | 107 | 47 | 527 | 193 |
| | (76.07%) | (23.92%) | (69.48%) | (30.51%) | (73.19%) | (26.8%) |
| 30750 | 386 | 0 | 152 | 0 | 729 | 1 (0.13%) |
| | (100%) | | (100%) | | (99.86%) | |
| 30852 | 386 | 0 | 156 | 0 | 732 | 4 (0.54%) |
| | (100%) | | (100%) | | (99.45%) | |
| 30870 | 298 | 72 | 118 | 28 | 592 | 114 |
| | (80.54%) | (19.45%) | (80.82%) | (19.17%) | (83.85%) | (16.14%) |
| 33038 | 303 | 73 | 125 | 27 | 587 | 127 |
| | (80.58%) | (19.41%) | (82.23%) | (17.76%) | (82.21%) | (17.78%) |
| 33186 | 387 | 1 (0.25%) | 156 | 0 | 733 | 1 (0.13%) |
| | (99.74%) | | (100%) | | (99.86%) | |
| 33220 | 384 | 0 | 154 | 0 | 727 | 1 (0.13%) |
| | (100%) | | (100%) | | (99.86%) | |
| 33463 | 330 | 0 | 156 | 0 | 661 | 1 (0.15%) |
| | (100%) | | (100%) | | (99.84%) | |
| 33734 | 390 | 2 (0.51%) | 156 | 0 | 741 | 3 (0.4%) |
| | (99.48%) | | (100%) | | (99.59%) | |
| 33761 | 395 | 1 (0.25%) | 156 | 0 | 745 | 1 (0.13%) |
| | (99.74%) | | (100%) | | (99.86%) | |
| 34067 | 324 (81%) | 76 (19%) | 144 | 12 | 656 | 100 |
| | | | (92.3%) | (7.69%) | (86.77%) | (13.22%) |
| 11972 | 319 | 1 (0.31%) | 144 | 0 | 651 | 7 (1.06%) |
| | (99.68%) | | (100%) | | (98.93%) | |
| 16643 | 92 (100%) | 0 | 0 | 0 | 180 | 2 (1.09%) |
| | | | | | (98.9%) | |
| 30671 | 94 | 10 | 150 | 4 (2.59%) | 370 | 14 |
| | (90.38%) | (9.61%) | (97.4%) | | (96.35%) | (3.64%) |
| 32977 | 384 | 8 (2.04%) | 148 | 0 | 717 | 9 (1.23%) |
| | (97.95%) | | (100%) | | (98.76%) | |
| 34180 | 313 | 77 | 137 | 13 | 615 | 119 |
| | (80.25%) | (19.74%) | (91.33%) | (8.66%) | (83.78%) | (16.21%) |

Of the 140 polymorphisms listed in Table 4, 14 were located in the 5′ UTR or promoter region, 89 in introns, three in the 3′ UTR, and 34 in the coding region, with 20 of these leading to amino acid changes (Table 4). The resequenced region of NPC1L1 spanned 20,094 bases, so that the average number of SNPs per kb was 0.083725 for common SNPs and 6.96725 over all SNPs, consistent with numbers reported over broader sets of genes (Crawford, et al., (2004) Am. J. Hum. Genet. 74:610-22).

Table 5 highlights the 24 SNPs selected from Table 4 that had minor allele frequencies (MAF)>4% detected in at least one ethnic group.

TABLE 5
24 NPC1L1 SNPs Having MAF > 4%

| | | Resequencing Cohort | | | EASE Cohort | | | |
|---|---|---|---|---|---|---|---|---|
| | | White | Hispanic | Black | White | Hispanic | Asian | Black |
| SNP Location | Source | (N = 198) | (N = 78) | (N = 99) | (N = 1003) | (N = 52) | (N = 39) | (N = 101) |
| g.-982G > C | Reseq | 3.1 | 0.6 | 5.1 | NG | NG | NG | NG |
| g.-762T > C^{a} | Reseq/rs2073548* | 0.3 | 8.3 | 3.1 | NG | NG | NG | NG |
| g.-133A > G^{a,b} | Reseq | 29.9 | 18.6 | 9.2 | 30.0 | 23.0 | 6.0 | 8.0 |
| g.-18C > A^{a,b} | Reseq | 18.1 | 7.5 | 6.0 | 16.0 | 5.0 | 5.0 | 5.0 |
| g.-1679C > G (L272L) | Reseq/rs2072183 | 21.9 | 28.3 | 17.9 | 22.0 | 22.0 | 35.0 | 20.0 |
| g.1680G > T^{a} | Reseq | 0.0 | 7.9 | 0.0 | NG | NG | NG | NG |
| g.1791G > T^{a} | Reseq | 0.0 | 0.0 | 4.0 | NG | NG | NG | NG |
| g.2023A > G (N387S)^{a,b} | Reseq | 0.0 | 0.0 | 5.1 | 0.001 | 0.0 | 0.0 | 12.0 |
| g.3237C > T^{b} | Reseq | 13.2 | 7.7 | 8.4 | 17.0 | 6.0 | 4.0^{c} | 8.0 |
| g.6893C > T^{a} | Reseq | 0.0 | 0.0 | 11.2 | NG | NG | NG | NG |
| g.9096T > G^{a} | Reseq | 0.0 | 0.6 | 5.0 | NG | NG | NG | NG |
| g.9202C > T^{a,b} | Reseq | 14.3 | 8.9 | 5.6 | 17.0 | 5.0 | 3.0 | 7.0 |
| g.9231G > A | Reseq | 4.6 | 3.8 | 6.6 | NG | NG | NG | NG |
| g.16124A > G^{a,b} | Reseq/rs1088837 | 24.5 | 8.3 | 10.4 | 25.0 | 19.0 | 6.0 | 22.0^{d} |
| g.18958T > G^{a,b} | Reseq | 23.7^{c} | 8.4 | 16.8 | 24.0 | 16.0 | 5.0 | 29.0^{d} |
| g.18975G > A | Reseq/rs4720470 | 4.9 | 10.4 | 4.6 | NG | NG | NG | NG |
| g.19224G > A^{b} | Reseq | 10.6 | 8.6 | 3.8 | 8.0 | 5.0 | 100.0 | 3.0 |
| g.19259T > C^{a,b} | Reseq | 23.1 | 1.9 | 15.2 | 25.0 | 18.0^{d} | 4.0 | 30.0^{d} |
| g.23825G > A^{a} | Reseq | 0.3 | 0.0 | 4.2 | NG | NG | NG | NG |
| g.25286A > C^{a} | Reseq/rs1315929 | 24.2^{c} | 30.5 | 8.0 | NG | NG | NG | NG |
| g.25453C > T (Y1264Y) | Reseq | 19.7 | 19.2 | 29.2 | NG | NG | NG | NG |
| g.27621T > C (V1269V)^{b} | Reseq | 19.5 | 17.1 | 13.0 | 23.0 | 12.0 | 4.0 | 7.0 |
| g.28650A > G^{b} | Reseq | 4.9 | 3.8 | 3.5 | 21.0^{d} | 17.0^{d} | 4.0 | 15.0^{c,d} |
| g.28763DEL^{b} | Reseq | 18.1 | 8.7 | 13.8 | 21.0 | 13.0 | 4.0 | 5.0^{d} |

^{a}Statistically significant differences in allele frequencies between at least two ethnicities in the resequencing cohort (p < 0.005)
^{b}Statistically significant differences in allele frequencies between at least two ethnicities in the EASE cohort (p < 0.005)
^{c}Statistically significant departure from Hardy-Weinberg Equilibrium (p < 0.01 using the Exact Test for HWE)
^{d}Statistically significant differences in allele frequencies between the resequencing and EASE cohorts in at least one ethnic group (p < 0.005)
*The “rs” number in column two refers to a SNP accession number previously reported in the NCBI SNP database.

### Example 2

### Linkage Disequilibrium (LD) Analysis of NPC1L1 Gene in the Resequencing Cohort

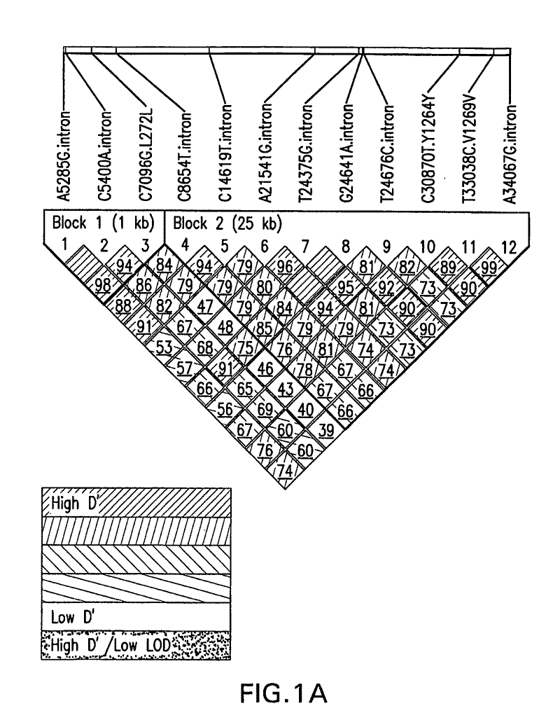

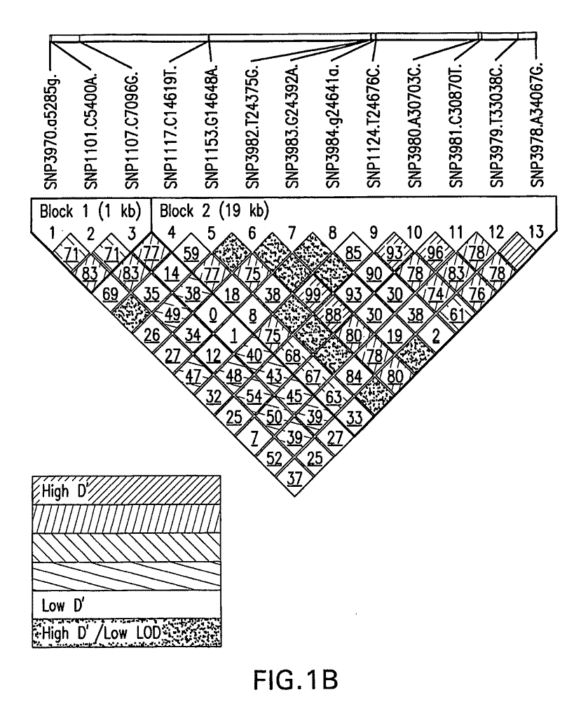

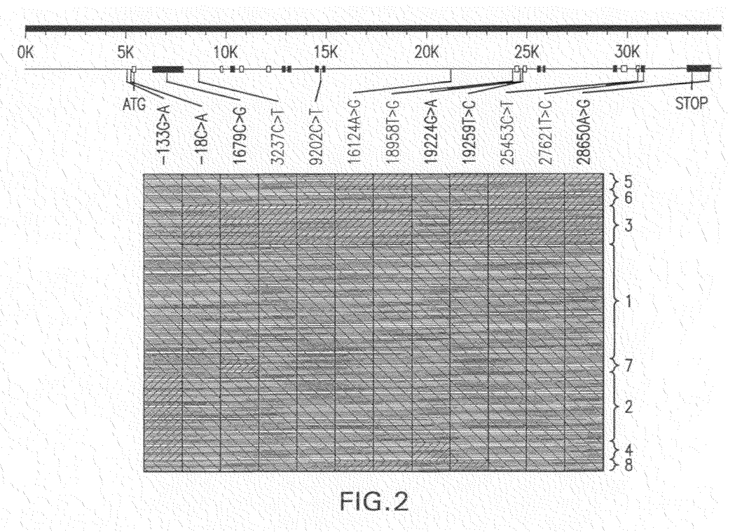

Hardy-Weinberg equilibrium was assessed on all individual polymorphisms using a standard contingency table comparing observed and predicted genotype frequencies, where predicted frequencies were estimated by the exact test procedure implemented in the Haploview software package (Barrett, et al., (2005) Bioinformatics, 25:263-5). Pairwise linkage disequilibrium values shown in FIG. 1A for all SNP pairs were computed using the Haploview program. Lewontin's disequilibrium coefficient (D′) was computed for all SNP pairs using the observed allele frequencies for each SNP. Haplotypes were inferred in the re-sequencing cohort using a Bayesian approach to haplotype reconstruction implemented in the PHASE v2.0 software package (Stephens, et al., (2001) Am. J. Hum. Genet., 68:978-89). SNPs with MAF>4% were used in the haplotype reconstruction process. Recombination hot spot intensity was computed using the Phase v2.0 software package, as previously described (Crawford, et al., (2004) Nat. Genet., 36:700-6). Using a slight variation of the method presented by Crawford et al., ((2004) Am. J. Hum. Genet., 74:610-22) to group haplotypes and SNPs according to allelic similarity, the eight most common haplotypes identified over each of the ethnic groups were identified. Haplotypes for all chromosomes observed were then clustered by similarity using an agglomerative hierarchical clustering procedure. Similarly, SNPs were clustered by allelic similarity using the same type of clustering procedure (FIG. 2). Tagging SNPs that distinguish among the common haplotypes (frequency>2%) were then identified visually from the resulting gray scale matrix plot in FIG. 2.

To determine if minor allele frequencies for each SNP were equivalent for all ethnic groups, the Pearson's χ² statistic was computed based on the expected number of minor alleles for each ethnic group, estimated by multiplying the number of individuals in an ethnic group by the fraction of minor alleles observed over all of the individuals in the cohort. Under the null hypothesis that the frequencies are the same across all ethnic groups, the Pearson's χ² statistic has an asymptotic χ² distribution with degrees of freedom equal to the number of ethnic groups minus 1. In cases where the minor allele frequency (MAF) for a given SNP in any of the ethnic groups was too small for the asymptotics to hold, permutation testing was performed, if possible, to estimate significances empirically. In such cases the permutation step consisted of randomly assigning individuals in a given cohort to genotypes for the SNP of interest, preserving the overall allele counts observed in the cohort, and then computing the Pearson's χ² statistic.

Strong LD blocks were not well defined for the different ethnic groups, despite having genotype information on over 350 individuals. FIG. 1A highlights the LD map for Caucasians from the resequencing cohort. Pairwise D′ values were high for only a few physically adjacent SNP pairs. The blocks highlighted in this figure were identified using the Four Gamete Rule (Wang, et al., (2002) Am. J. Hum. Genet., 71:1227-34), but the threshold for the minimum frequency for the fourth gamete had to be set to 0.05 to realize this structure. Interestingly, this gene had a recombination hot spot intensity of 45, computed using the Phase v2.0 software package (Stephens, et al., (2001) Am. J. Hum. Genet., 68:978-89), as previously described (Crawford, et al., (2004) Am. J. Hum. Genet., 74:610-22). This suggests NPC1L1 has a significantly increased rate of recombination compared to other genes. Haplotypes were also inferred using a Bayesian approach to haplotype reconstruction implemented in the PHASE v2.0 software package (Stephens, et al., (2001) Am. J. Hum. Genet., 68:978-89). SNPs with MAF>4% were used in the haplotype reconstruction process. The number of haplotypes inferred in the African-American, Caucasian, and Hispanic populations was 139, 156, and 189, respectively. This number is significantly above the average numbers reported in surveys over larger sets of genes (Crawford, et al., (2004) Am. J. Hum. Genet., 74:610-22), most likely highlighting the increased diversity achieved from the larger number of samples and the putative increased rate of recombination in this gene.

The number of common haplotypes (>5% frequency) in the African-American, Caucasian, and Hispanic populations was 2, 4, and 4, respectively, where these common haplotypes explained 53%, 57%, and 48% of the chromosomes in these same populations. The extent of haplotype diversity was assessed in several ways. First, of the 345 haplotypes inferred in the combined population, 26 were shared between all three populations. The percentage of chromosomes in each population explained by these 26 haplotypes was 73% in the African-American population, 67% in the Caucasian population, and 62% in the Hispanic population, with the African-American and Caucasian populations having the greatest percentage of chromosomes explained by common haplotypes (80%). There was little variation in these ratios if subsets of individuals were resampled from the different populations and haplotypes were inferred from those subsets, indicating that the larger numbers of individuals did not significantly increase the diversity of common haplotypes beyond what would have been achieved using a smaller cohort, as expected (Kruglyak and Nickerson (2001) Nat. Genet., 27:234-6).

### Example 3

### Association of NPC1L1 Polymorphisms with Treatment Responses to Dual (Add-On) Drug Therapy with Ezetimibe and Statins

The data in this example show that several NPC1L1 SNPs and haplotypes are significantly associated with the level of response of a subject to ezetimibe add-on to statin treatment. Genotyping assays were developed for a number of novel and known common variants with minor allele frequencies greater than 4% that were identified in Example 1. Genetic association analysis was performed with these SNPs in a clinical trial cohort (EASE), described below, to assess whether DNA sequence variants in NPC1L1 are associated with changes in the levels of a variety of plasma cholesterol components in hypercholesterolemia patients in response to pharmacotherapy with ezetimibe and statins as compared to patients treated with a statin and placebo.

The EASE Cohort

To study whether variations in NPC1L1 were associated with response to ezetimibe added to statin therapy, a study population was derived from the Ezetimibe Add-On to Statin for Effectiveness (EASE) Trial (Pearson et al., (2005) Mayo Clinic Proceedings, In Press). The EASE trial was a community-based, randomized, double-blind, placebo controlled study to evaluate the effects of six weeks of ezetimibe, 10 mg/day, added on to a stable regimen of statin therapy, on lipid biomarkers in hypercholesterolemic patients whose LDL-C levels exceeded the National Cholesterol Education Program (NCEP) Adult Treatment Panel (ATP) m guidelines for their coronary heart disease (CHD) risk category. At enrollment, patients taking a stable dose of statin (any dose, any brand) and following a NCEP Step 1 diet or similar cholesterol-lowering diet for at least six weeks prior to entry into the study were randomized to either the ezetimibe (n=2020, 2009 received the treatment) or placebo (n=1010, 1009 received the treatment) arm. From the ezetimibe group, 1208 patients provided consent for genomic analysis and were included in this study. A series of clinical measures corresponding to various cardiovascular risk factors were measured from samples obtained from all trial participants and are summarized by Pearson et al., supra.

SNP Selection and Genotyping in the EASE Cohort

Twenty one SNPs from Table 4 (Example 1) were converted to valid genotyping assays, thirteen of which had allele frequencies greater than 2% in all EASE sub-populations. TaqMan Allelic Discrimination assays (Livak, (1999) Genet. Anal. 14:143-49) were performed using Primer Express software and the Assay-by-Design service offered by Applied Biosystems (Foster City, Calif.). Table 6 shows the PCR primers and fluorogenic probe sequences used to perform the allelic discrimination assays on the thirteen selected NPC1L1 SNPs having an allele frequency of greater than 2% in all EASE sub-populations. All probe/primer sets were designed to function using universal reaction and cycling conditions.

TABLE 6

Primer and probe sequences for the TaqMan allele discrimination
assays used to genotype NPC1L1 SNPs in the EASE cohort

| | | | VIC Probe Sequence | FAM Probe |
|---|---|---|---|---|
| | | | with Quencher for | Sequence with |
| NPC1L1 | Forward PCR | Reverse PCR | Major Allele | Quencher for Minor |
| SNP | Primer Sequence | Primer Sequence | Detection | Allele Detection |
| g.−133A > G | CAGTGGGAGTGGTGGA | CTGGCCTGACTGGGTTA | CCAATGAGGCTGAGCC | CCAATGAGGCCGAGCC |
| | TCATTAAC | GG | SEQ ID NO: 106 | SEQ ID NO: 107 |
| | SEQ ID NO: 104 | SEQ ID NO: 105 | | |
| G.−18C > A | GGCCTGGCCTGGCT | CGCCATCCCAGGTCTGG | CCGCTGACCCCTTC | CGCTGAACCCTTC |
| | SEQ ID NO: 108 | SEQ ID NO: 109 | SEQ ID NO: 110 | SEQ ID NO: 111 |
| g.1679C > G | GCATCCTGTCCTGCCAT | GCATCTGGCCCAGGTA | CCCTCGACTCCACC | CCCTGGACTCCACC |
| | AGC | GAA | SEQ ID NO: 114 | SEQ ID NO: 115 |
| | SEQ ID NO: 112 | SEQ ID NO: 113 | | |
| g.2023A > G | CCCGTGGAGCTGTGGTC | GAAATGCTGGTCATGG | CCCCCAACAGCCAA | CCCCAGCAGCCAA |
| | SEQ ID NO: 116 | AAAGCT | SEQ ID NO: 118 | SEQ ID NO: 119 |
| | | SEQ ID NO: 117 | | |
| g.3237C > T | CTGACCTTACAGACCCT | CCAATCCAGTGGTTCTC | CCCTTAGGCGTCCTG | CCCTTAGGCATCCTG |
| | GGAAAG | AAAGTGT | SEQ ID NO: 122 | SEQ ID NO: 123 |
| | SEQ ID NO: 120 | SEQ ID NO: 121 | | |
| g.9202C > T | CTCGAGGTGTTGTGGTG | GCGAGGTCCCCACCTA | CTGCTCTCGTG126TGGT | CCTGCTCTCATGTGGTT |
| | AGT | GT | T | SEQ ID NO: 127 |
| | SEQ ID NO: 124 | SEQ ID NO: 125 | SEQ ID NO: 126 | |
| g.16124A > G | CCTATTGGAGTTTATTG | GCGAGGTCCCCACCTA | CAAATAATCTCACTTCC | ATAATCTCGCTTCCCC |
| | AGTTTCTTGAATGTTTA | GTAGACCAAAATATGA | CC | SEQ ID NO: 131 |
| | TATTC | ATT | SEQ ID NO: 130 | |
| | SEQ ID NO: 128 | SEQ ID NO: 129 | | |
| g.18958T > G | TGTGTGTACCTTCGAGA | TGAGCTTTGGTTCGCTA | TAAAGGGCTCAATCCA | CTAAAGGGCTCACTCC |
| | GTGTGA | TGCA | SEQ ID NO: 134 | A |
| | SEQ ID NO: 132 | SEQ ID NO: 133 | | SEQ ID NO: 135 |
| g.19224G > A | GAGTTCCCTGAGCAGT | GACAGGGATAGAACAT | CTGGCCCGCCCCAA | CTGGCCCACCCCAA |
| | GAGTT | CAGGAAGAG | SEQ ID NO: 138 | SEQ ID NO: 139 |
| | SEQ ID NO: 136 | SEQ ID NO: 137 | | |
| g.19259T > C | CCCAAACCCCAGCCTA | GACAGGGATAGAACAT | CTGTTTGAGTCCCTCCA | CTGTTTGAGTCCCCCCA |
| | CTC | CAGGAAGAG | GT | GT |
| | SEQ ID NO: 140 | SEQ ID NO: 141 | SEQ ID NO: 142 | SEQ ID NO: 143 |
| g.25453C > T | GGTCTTCCTGCCCGTCA | AGCATAATCATGACAG | TCACCCACGTAGCTGA | TCACCCACATAGCTGA |
| | TC | TCTGGTAGGA | SEQ ID NO: 146 | SEQ ID NO: 147 |
| | SEQ ID NO: 144 | SEQ ID NO: 145 | | |
| g.27621T > C | TCTGACTGTGGTTCTCT | CTCCTCAGCCCGCTTCT | CCGGGTTAACGTCAG | CCGGGTTGACGTCAG |
| | GTCTCT | G | SEQ ID NO: 150 | SEQ ID NO: 151 |
| | SEQ ID NO: 148 | SEQ ID NO: 149 | | |
| g.28650A > G | GCCCAACCCGAGCTTTT | CACAGAGCCAGGATCT | CCAGAAGCATGAACTG | CAGAAGCGTGAACTG |
| | G | TCATCTC | SEQ ID NO: 154 | SEQ ID NO: 155 |
| | SEQ ID NO: 152 | SEQ ID NO: 153 | | |

After PCR amplification, an endpoint plate read using Applied Biosystems 7900 HT Sequence Detection System (SDS) was performed. Genotypes with quality scores below 95% were repeated.

The twenty one selected SNPs were genotyped in 1,208 individuals participating in the ezetimibe+statin treatment arm of the EASE trial. A series of clinical measures corresponding to various cardiovascular risk factors were taken on all trial participants (Tables 4a-d). Thirteen selected SNPs genotyped in the EASE cohort were confirmed as having common allele frequencies in this cohort, i.e., an allele frequency of greater than 2% in all EASE sub-populations. A greater percentage of SNPs had significantly different allele frequencies among ethnic groups in the EASE cohort as compared to the resequencing cohort. This could reflect the increased power in the larger EASE cohort to make such detections (see Table 5).

Linkage Disequilibrium Analysis of the EASE Cohort

Given the large number of individuals genotyped in the EASE cohort, the LD structure through the NPC1L1 gene was more apparent. The pairwise D′ values (FIG. 1B) were high through the LD blocks identified in the resequencing cohort. With the exception of SNP g. 1680G>T, the D′ values were reasonably high for all SNP pairs through the entire length of the gene, suggesting that the highlighted LD blocks were not as well defined and that all SNPs were in LD to some degree. Haplotypes for the thirteen SNPs genotyped in the EASE cohort and with minor allele frequencies >=4% in all ethnic groups were inferred using the PHASE v2.0 software package at the default settings (Stephens, et al., (2001) Am. J. Hum. Genet., 68:978-89). Using a slight variation of the method presented by Crawford et al. ((2004) Am. J. Hum. Genet., 74:610-22) to group haplotypes and SNPs according to allelic similarity, the eight most common haplotypes identified over each of the ethnic groups were identified. Haplotypes for all chromosomes observed were then clustered by similarity using an agglomerative hierarchical clustering procedure (FIG. 2). Similarly, SNPs were clustered by allelic similarity using the same type of clustering procedure. Six tagging SNPs were identified that were capable of representing the eight different common haplotypes that explain more than 80% of the haplotype diversity in the EASE cohort. These six tagging SNPs were used to characterize genetic association between NPC1L1 and LDL-C response to treatment with ezetimibe.

Genetic Associations Testing

Participants in the EASE trial had a mean (SD) age of 62.0 (11.3), with 1,522 (52.3%) males and 1,386 females (47.7%). The mean (SD) for total plasma cholesterol, HDL cholesterol (HDL-C), and LDL cholesterol (LDL-C) was 211.0 (34.9), 48.6 (11.5), and 129.1 (30.0) mg/dL, respectively (Pearson et al., supra). Subjects in the ezetimibe group had a significantly greater reduction in LDL-C compared to placebo treated subjects (25.8% v. 2.7%, p<0.001). The distribution of these measurements was similar in the subjects enrolled in this genetic study (Pearson et al., supra). Baseline clinical measures listed in Pearson et al., supra were significantly correlated to each other (Table 7) and correlated with LDL-C response to treatment with ezetimibe (Table 8), defined as the percent reduction from baseline in LDL-C levels after 6 weeks of ezetimibe added to concomitant statin therapy. Age, race, sex, and BMI were not statistically significantly predictive of ezetimibe response. A general linear model was used to assess whether these LDL-C response predictive baseline variables were significantly associated with any of the six tagging SNPs identified in the NPC1L1 gene. No significant associations were found between these response predictive variables and any of the tagging SNPs.

TABLE 7
Correlation of baseline clinical measurements

| | % | | | | | | | | | | |
|---|---|---|---|---|---|---|---|---|---|---|---|
| | Change | | | | | | | | | | |
| | from | | | | | | | | | Total- | |
| | Baseline | | | | | Non- | | | LDL-HDL | C:HDL-C | |
| | LDL-C | LDL-C | TG | HDL-C | Total-C | HDL-C | APO-AI | APO-B | C ratio | ratio | Hemog-A1c |
| LDL-C | −0.26 | 1.00 | | | | | | | | | |
| | <.0001 | | | | | | | | | | |
| | 1003 | 1003 | | | | | | | | | |
| TG | 0.07 | 0.03 | 1.00 | | | | | | | | |
| | 0.03 | 0.34 | | | | | | | | | |
| | 1003 | 1003 | 1003 | | | | | | | | |
| HDL-C | −0.03 | 0.09 | −0.32 | 1.00 | | | | | | | |
| | 0.32 | 0.00 | <.0001 | | | | | | | | |
| | 1003 | 1003 | 1003 | 1003 | | | | | | | |
| Total-C | −0.19 | 0.88 | 0.36 | 0.27 | 1.00 | | | | | | |
| | <.0001 | <.0001 | <.0001 | <.0001 | | | | | | | |
| | 1003 | 1003 | 1003 | 1003 | 1003 | | | | | | |
| Non- | −0.18 | 0.88 | 0.49 | −0.07 | 0.94 | 1.00 | | | | | |
| HDL-C | <.0001 | <.0001 | <.0001 | 0.03 | <.0001 | | | | | | |
| | 1003 | 1003 | 1003 | 1003 | 1003 | 1003 | | | | | |
| APO-AI | −0.01 | 0.10 | −0.06 | 0.87 | 0.35 | 0.06 | 1.00 | | | | |
| | 0.66 | 0.00 | 0.06 | <.0001 | <.0001 | 0.06 | | | | | |
| | 982 | 982 | 982 | 982 | 982 | 982 | 982 | | | | |
| APO-B | −0.16 | 0.82 | 0.43 | −0.11 | 0.86 | 0.92 | 0.05 | 1.00 | | | |
| | <.0001 | <.0001 | <.0001 | 0.00 | <.0001 | <.0001 | 0.10 | | | | |
| | 982 | 982 | 982 | 982 | 982 | 982 | 982 | 982 | | | |
| LDL:HDL-C | −0.15 | 0.66 | 0.27 | −0.64 | 0.45 | 0.69 | −0.56 | 0.68 | 1.00 | | |
| ratio | <.0001 | <.0001 | <.0001 | <.0001 | <.0001 | <.0001 | <.0001 | <.0001 | | | |
| | 1003 | 1003 | 1003 | 1003 | 1003 | 1003 | 982 | 982 | 1003 | | |
| Total-C:HDL- | −0.08 | 0.48 | 0.57 | −0.71 | 0.42 | 0.69 | −0.56 | 0.66 | 0.93 | 1.00 | |
| C ratio | 0.01 | <.0001 | <.0001 | <.0001 | <.0001 | <.0001 | <.0001 | <.0001 | <.0001 | | |
| | 1003 | 1003 | 1003 | 1003 | 1003 | 1003 | 982 | 982 | 1003 | 1003 | |
| Hemog- | −0.20 | 0.03 | 0.08 | −0.11 | 0.03 | 0.07 | −0.11 | 0.08 | 0.08 | 0.09 | 1.00 |
| A1c | 0.00 | 0.56 | 0.11 | 0.04 | 0.62 | 0.21 | 0.04 | 0.15 | 0.12 | 0.08 | |
| | 353 | 353 | 353 | 353 | 353 | 353 | 347 | 347 | 353 | 353 | 353 |

TABLE 8
Tagging SNPs tested for association to LDL-C response to ezetimibe treatment using
baseline LDL-C as a covariate in the analysis.

| | | | | | Extreme Responder association with SNP | | | |
|---|---|---|---|---|---|---|---|---|
| | | | | | (Percent change in LDL-C either less than the 10^{th} or | | | |
| | | | | | greater than the 90^{th} percentile | | | |
| | | LR | | | N = 239 | | | |
| | SNP | P-value | | | General | | | LR P-value for |
| | P- | for | Least-Squares | | Association | | Baseline | Least-Squares |
| SNP | value | Baseline | (adjusted Mean) | | Test P-value | SNP P-value | P-value | (adjusted) Mean |
| g.-133A > G | 0.0793 | <0.0001 | A/A | −25.9 | 0.072 | 0.18 | <0.0001 | |
| | | | A/G | −24.8 | | | | |
| | | | G/G | −21.9 | | | | |
| g.-18C > A* | 0.0035 | <0.0001 | A/A | −26.5 | 0.0005 | 0.0019 | <0.0001 | A/A n = 0 |
| | (0.021) | | C/A | −27.9 | (0.003) | (0.0114) | | C/C: −17.8 |
| | | | C/C | −24.2 | | | | Odds Ratio = 2.94- |
| | | | | | | | | CI = (1.59, 5.44) |
| g.1679C > G | 0.149 | <0.0001 | C/C | −24.5 | 0.012 | 0.1548 | <0.0001 | |
| | | | C/G | −26 | | | | |
| | | | G/G | −27.9 | | | | |
| g.19224G > A | 0.763 | <0.0001 | A/A | −28.6 | 0.6 | 0.841 | <0.0001 | |
| | | | G/A | −25.8 | | | | |
| | | | G/G | −25 | | | | |
| g.19259T > C | 0.836 | <0.0001 | C/C | −26.2 | 0.846 | 0.797 | <0.0001 | |
| | | | T/C | −25.2 | | | | |
| | | | T/T | −25 | | | | |
| g.28650A > G | 0.053 | <0.0001 | A/A | −24.3 | 0.101 | 0.243 | <0.0001 | |
| | | | A/G | −26.6 | | | | |
| | | | G/G | −28 | | | | |

*As in Table 9 - Given the response counts - CI = 95% confidence interval

Genetic association analysis was carried out in the EASE cohort with LDL-C response to ezetimibe treatment considered as the primary outcome variable. Individual SNPs, haplotypes, and haplotype combinations were the principal explanatory variables used in the analyses. General linear models were used to estimate the effects of genotypes, haplotypes, and diplotypes on the LDL-C response phenotype. Baseline LDL-C levels, sex, age, and race were investigated to determine if they gave rise to significant effects. Baseline LDL-C levels associated with significant effects in all models and were therefore included in all analyses. However the effects of the SNPs on the percent change from baseline remained the same regardless of including baseline value in the model or not. Since there was no association between any of the tagging SNPs and baseline LDL-C values, we report the p-values for models only including the SNPs as predictor variables.

Association of response of LDL-C levels to treatment with ezetimibe and NPC1L1 SNPs was tested in a general linear model regression framework. Table 9 summarizes the association results for the six tagging SNPs identified in Table 5. In Table 9, the first two columns report results for the linear model implemented in software program SAS PROC GLM (SAS Institute, Inc.). The outcome is the percent change from baseline LDL-C and the SNP is the predictor, modeled as three categories. Similarly, columns 8 and 9 of Table 9 show the results for the same model, including only the subjects in the extreme tails for the percent change in LDL-C distribution. Columns 4 through 9 provide test results in the extreme responders of the treated arm of the EASE cohort, as described in the text. The p-value is the general association p-value obtained from the SAS software procedure PROC FREQ. If a significant p-value was achieved for association between response and SNP genotype (at the 0.05 level), the Bonferroni-corrected p-value is given in parentheses.

TABLE 9
Tagging SNPs tested for association to LDL-C response to ezetimibe treatment.

| | | | | Extreame Responder association with SNP | | |
|---|---|---|---|---|---|---|
| | | | | (Percent change in LDL-C either less than the 10^{th} or | | |
| | | | | greater than the 90^{th} percentile) | | |
| EASE cohort association analysis | | | | N = 239 | | |
| (N = 1195) | | | | General | | |
| | | Least-Squares | | Association | | |
| | SNP | adjusted Mean | | Test P- | SNP | Least-Squares |
| SNP | P-value | (n/Std error) | | value | P-value | (adjusted) Mean (n,/Stderr) |
| g.-133A > G | 0.142 | A/A | −25.92 (629/0.68) | 0.072 | 0.093 | |
| | | A/G | −24.58 (477/0.78) | | | |
| | | G/G | −22.56 (89/1.80) | | | |
| g.-18C > A* | 0.0043 | A/A | −27.27 (22/3.61) | 0.0005 | 0.0003 | n = 0 |
| | (0.026) | C/A | −27.85 (298/0.98) | (0.003) | (0.0018) | −33.98 (62/4.05) |
| | | C/C | −24.16 (875/0.57) | | | −16.85 (177/2.40) |
| | | | | | | Odds ratio** = 2.94- |
| | | | | | | CI = 1.59, 5.44) |
| g.1679C > G | 0.129 | C/C | −24.50 (723/0.63) | 0.012 | 0.028 | C/C: −17.49 (143/2.71) |
| | | C/G | −25.77 (417/0.83) | (0.072) | (0.170) | C/G: −25.16 (85/3.51) |
| | | G/G | −28.77 (55/2.29) | | | G/G: −40.93 (11/9.76) |
| g.19224G > A | 0.643 | A/A | −31.64 (/6.94) | 0.597 | 0.710 | |
| | | G/A | −25.07 (/1.34) | | | |
| | | G/G | −25.11 (/0.53) | | | |
| g.19259T > C | 0.807 | C/C | −26.38 (/1.98) | 0.846 | 0.598 | |
| | | T/C | −25.01 (/0.82) | | | |
| | | T/T | −25.07 (/0.64) | | | |
| g.28650A > G | 0.108 | A/A | −24.40 (/0.60) | 0.101 | 0.079 | |
| | | A/G | −26.50 (/0.89) | | | |
| | | G/G | −27.19 (/2.65) | | | |

**Given the response counts - CI = 95% confidence interval

SNP g.−18C>A, located 18 nucleotides upstream of the initiating ATG of the NPC1L1 coding sequence was found to be significantly associated with LDL-C response to ezetimibe treatment in the EASE cohort (p-value=0.0043). Patients homozygous for the common allele of g.−18C>A (n=875/1195; 73.2%) had a mean LDL-C change of 24.2% from baseline compared to 27.8% for patients heterozygous for the minor allele (298/1195; 25.0%), a 15% increased response. Individuals homozygous for the minor allele (n=22/1195; 1.8%) had a mean change in LDL-C of 27.3%, not significantly different from the heterozygotes. As indicated in Table 9, the association to SNP g.−18C>A was the only association that remained significant after conservative correction for all six SNPs tested when the analysis included the entire EASE population. In addition to g.−18C>A, one additional SNP (g. 1679C>G) was significantly associated to LDL-C response before correction for multiple testing (p-value=0.012).

Because Caucasians were the dominant ethnicity represented in the EASE cohort (1003/1195; 83.9%), this analysis was repeated using only the Caucasian subjects (Table 10). The association between LDL-C response and g.−18C>A in the Caucasian only subset of EASE was again found to be statistically significant (Table 10).

TABLE 10
Tagging SNPs tested for association to LDL-C response to ezetimibe treatment:
Caucasian ethnic subgroup.

| | | | | Extreme Responder association with SNP | | | | | |
|---|---|---|---|---|---|---|---|---|---|
| | | | | (Percent change in LDL-C either less than the 10^{th} or | | | | | |
| | | | | greater than the 90^{th} percentile | | | | | |
| | | Least-Squares | | N = 239 | | 95% CI | 95% CI | SNP | Least-Squares |
| | SNP | adjusted Mean | | General Association | Odds | Lower | Upper | P- | (adjusted) |
| SNP ID | P-value | (Std error) | | Test P-value | Ratio | Bound | Bound | value | Mean (Stderr) |
| g.-133A > G | 0.062 | A/A | −26.59 (0.73) | 0.090 | | | | 0.109 | |
| | | A/G | −24.78 (0.77) | | | | | | |
| | | G/G | −22.80 (1.72) | | | | | | |
| g.-18C > A* | 0.0025 | A/A | −27.19 (3.49) | 0.006 | 2.36 | 1.27 | 4.39 | 0.0011 | n = 0 |
| | (0.015) | C/A | −28.22 (0.96) | (0.036) | | | | (0.0066) | −32.81 (3.83) |
| | | C/C | −24.34 (0.60) | | | | | | −17.54 (2.57 |
| g.1679C > G | 0.111 | C/C | −24.66 (0.65) | 0.056 | | | | 0.057 | −18.38 (2.79 |
| (L272L) | | C/G | −26.58 (0.85) | | | | | | −26.61 (3.58) |
| | | G/G | −28.18 (2.51) | | | | | | −39.00 (10.81) |
| g.19224G > A | 0.560 | A/A | −31.64 (6.56) | 0.576 | | | | 0.541 | |
| | | G/A | −24.81 (1.31) | | | | | | |
| | | G/G | −25.56 (0.55) | | | | | | |
| g.19259T > C | 0.954 | C/C | −24.95 (2.01) | 0.504 | | | | 0.860 | |
| | | T/C | −25.61 (0.84) | | | | | | |
| | | T/T | −25.44 (0.67) | | | | | | |
| g.28650A > G | 0.103 | A/A | −24.63 (0.64) | 0.121 | | | | 0.0781 | |
| | | A/G | −26.90 (0.87) | | | | | | |
| | | G/G | −26.52 (2.57) | | | | | | |

*Statistically significant association to LDL-C response phenotype (p < 0.01)

Interestingly, allele frequencies for five SNPs in the Black ethnic group of the EASE cohort were significantly different from the corresponding frequencies in the resequencing cohort, potentially indicating different population substructures between these two groups. In addition, the allele frequencies for SNP g.28650A>G in the resequencing cohort (4.9% in the whites for example) differed significantly from those in the EASE cohort (21% in whites, p=6.7×10⁻¹⁴). This bias may reflect an association with response to statin therapy, given one of the requirements for enrolling EASE participants was failure to meet low-density lipoprotein cholesterol (LDL-C) lowering goals while on a statin therapy, and given no association between this SNP g.28650A>G and cholesterol baseline values was observed. Alternately, this may reflect an association to hypercholesterolemia in that the EASE cohort subjects were all dyslipidemic, while the resequencing cohort were population controls presumably having a normal distribution of cholesterol metabolism.

Extreme Responder Analysis

To further explore the association between g.−18C>A and lipid responses to ezetimibe treatment, the most extreme responders in the EASE cohort, defined as the upper and lower 10^{th} percentile of LDL-C responders to ezetimibe treatment were examined. Table 9 highlight the association analysis results for these extreme responders. Association to LDL-C response was found to be even more significant in the extreme responder subgroup compared to all treated trial participants (Table 8, p-value=0.0003 vs. 0.0043). Patients homozygous for the common allele in the extreme responders (176/239 individuals or 73.6%) had a mean LDL-C percent response of 16.8%, while the heterozygotes had a mean percent response of 33.98%, a 100% increase in efficacy.

Given the significant association of SNP g.−18C>A to LDL-C response and the two SNPs flanking this SNP in LD block 1 shown in FIG. 1B, all 3-SNP haplotypes (Table 11) and diplotypes (Table 11) were examined for association to LDL-C response in the extreme responders defined above. The haplotypes for Tables 11 and 12 were inferred using the statistical software package SAS (SAS Institute, Inc., Cary, N.C.), in the EASE cohort.

Table 11 shows association test results for the five most common three-SNP haplotypes constructed from SNPs g.−133A>G, g.−18C>A, g.1678C>G tested in the extreme responders. A haplotype trend test was used to determine whether individuals carrying different numbers of a given haplotype differed significantly with respect to response. The third column represents the coding used for classifying individuals as carrying 0, 1, or 2 copies of the haplotype. Counts were treated as categorical variables in the general linear model. In Table 10, the number of copies of the haplotypes (estimated in SAS program PROC HAPLOTYPES) are modeled as categorical outcomes, again using the SAS software PROC GLM.

TABLE 11
Association Results for the Five Most Common Three-SNP Haplotypes

| 3-SNP Haplotypes | | | | |
|---|---|---|---|---|
| g.−133A > | | | Adjusted Least | |
| G-g.−18C > A- | | | Squares | |
| g.1678C > G | P-value* | Counts | Mean (stderr) | P-Value** |
| A-A-C | 0.280 | 235 | | |
| | | 4 | | |
| A-A-G | 0.0005 | 181 | −17.32 (2.38) | 0.0008 |
| | | 58 | −33.69 (4.21) | |
| A-C-C | 0.225 | 45 | | |
| | | 129 | | |
| | | 65 | | |
| A-C-G | 0.115 | 197 | | |
| | | 36 | | |
| | | 6 | | |
| G-C-C | 0.062 | 139 | −24.51 (2.75) | 0.0342 |
| | | 92 | −18.62 (3.38) | |
| | | 8 | 3.93 (11.45) | |

*Model including all haplotypes
**Model including only corresponding haplotype
P-value for the F-test where null hypothesis is mean response for AAG carriers in the low responding group is equal to mean response for AAG carriers in the high responding group.

Table 12 shows the diplotype counts and mean LDL-C response rates as determined by treating diplotypes as categorical variables and fitting LDL-C response to a general linear model using the extreme responder data set.

TABLE 12
Diplotype Counts and LDL-C Response Rates

| | | | | Diplotype | Adjusted Least | |
|---|---|---|---|---|---|---|
| | | | | Frequency | Squares | |
| | Diplotype | | Count | (%) | Mean (stderr) | |
| Higher | A-A-C | A-C-C | 3 | 2.50 | −38.20 | (15.72) |
| Responders | A-A-G | A-C-C | 26 | 21.67 | −34.80 | (5.17) |
| | A-A-G | A-C-G | 4 | 3.33 | −40.29 | (14.06) |
| | A-A-G | G-C-C | 10 | 8.33 | −29.06 | (7.86) |
| | A-C-C | A-C-C | 32 | 26.67 | −21.78 | (3.90) |
| | A-C-C | A-C-G | 7 | 5.83 | −4.60 | (6.70) |
| | A-C-C | G-C-C | 26 | 21.67 | −14.60 | (3.87) |
| | A-C-G | A-C-G | 5 | 4.17 | −41.46 | (12.84) |
| | A-C-G | G-C-C | 5 | 4.17 | −24.20 | (10.48) |
| | G-C-C | G-C-C | 1 | 0.83 | 3.93 | (11.12) |
| | G-C-C | G-C-G | 1 | 0.83 | −66.96 | (31.44) |

| Total | | | 120 | 100.00 |
|---|---|---|---|---|
| Lower | A-A-C | A-C-C | 1 | 0.84 |
| Responders | A-A-G | A-C-C | 11 | 9.24 |
| | A-A-G | A-C-G | 1 | 0.84 |
| | A-A-G | G-C-C | 6 | 5.04 |
| | A-C-C | A-C-C | 33 | 27.73 |
| | A-C-C | A-C-G | 15 | 12.61 |
| | A-C-C | G-C-C | 40 | 33.61 |
| | A-C-G | A-C-G | 1 | 0.84 |
| | A-C-G | G-C-C | 4 | 3.36 |
| | G-C-C | G-C-C | 7 | 5.88 |

| | | |
|---|---|---|
| Total | 119 | 100.00 |

Linear model fit including all diplotypes as categorical variables: P-value = 0.0002

In Table 12, all pairs of haplotype-pair categories are modeled as a categorical outcome, with ten degrees of freedom, also in SAS program PROC GLM. Table 12 presents the counts for these categories for the high and low responders, the categorical test general association p-value, and also the p-values from the model with percent change from LDL-C baseline value as outcome.

Carriers of the [A(−133), A(−18), G(1679)] haplotype (designated A-A-G in Tables 11 and 12) containing the minor allele of the SNP g.−18C>A had significantly improved LDL-C response compared to non-carriers (p-value=0.0008). This pattern was apparent in both the analysis of the haplotypes and the analysis of the haplotype pairs (some of the resulting cell counts in the analysis of the diplotypes were small and may have influenced the test statistics). No individual haplotype or diplotype associations were found to be more significantly associated with response than SNP g.−18C>A. Further, none of the seven non-tagging SNPs that were genotyped in the EASE cohort were found to be as significantly associated with LDL-C response as SNP G.−18C>A. In addition, none of the eight most common haplotypes identified in the EASE cohort were found to be as significantly associated with LDL-C response as SNP G.−18C>A and the [A(−133), A(−18), G(1679)] haplotype. Importantly, SNP G.−18C>A and the [A(−133), A(−18), G(1679)] haplotype remained significantly associated to LDL-C response after adjusting LDL-C response levels for baseline LDL-C levels. Note that LDL-C baseline values were not found to be significantly associated with SNP G.−18C>A or any of the other 5 tagging SNPs tested.

### SUMMARY

This example presents a detailed characterization of DNA variations in the NPC1L1 gene, a gene encoding a protein in the ezetimibe sensitive pathway. Data is presented demonstrated that common polymorphisms in this gene are significantly associated with LDL-C response to ezetimibe treatment, but not to baseline LDL-C levels. Over 140 polymorphisms were identified in NPC1L1 in the re-sequencing cohort (Example 1), with 25 previously represented in dbSNP. One common SNP, g.−18C>A, was identified that was significantly associated with a 15% increased reduction in LDL-C levels compared to the homozygous major allele following six weeks of treatment with ezetimibe added to ongoing statin therapy. In the subset of extreme LDL-C responders to this treatment, the association for the g.−18C>A SNP was accentuated to a 100% increased reduction in LDL-C. The primary association (over all subjects) remained significant after conservative correction for all SNPs considered in the analysis and after accounting for age, sex, and baseline LDL-C covariates. In addition, G.28650A>G, which maps to the 3′ end of NPC1L1, demonstrated minor allele frequencies in all three ethnicities of the re-sequencing cohort that were significantly reduced compared to the corresponding minor allele frequencies in the EASE cohort. This reduction was confirmed by re-genotyping the re-sequencing cohort with the same assay as the one used in the EASE cohort.

Ezetimibe lowers LDL-C by blocking the small intestinal cholesterol transporter, NPC1L1. As a monotherapy ezetimibe lowers LDL-C by approximately 18% (Knopp, et al., (2003) Int. J. Clin. Pract., 57:363-8). When co-administered with a statin the incremental reduction attributable to ezetimibe is approximately 14-15%. When added to ongoing statin therapy in patients on a stable dose of statins as studied in EASE, ezetimibe reduces LDL-C by an additional ˜23% as compared with addition of placebo to ongoing statin therapy (Pearson, et al., (In Press) Mayo Clinic Proceedings). At a similar statin dose of 20 mg, the addition of ezetimibe 10 mg (when administered as the combination vytorin tablet) further decreases the LDL-C change from baseline from 34% to 52%. Cholesterol response to lipid lowering therapies (statins and ezetimibe) is variable. A recent study demonstrated that a SNP with an allele frequency of ˜5% in the HMG CoA Reductase gene associates with a 19% lesser response to pravastatin (Chasman et al., (2004) Jama, 291:2821-7). This observation suggests the presence of genetic predictors of response to lipid lowering therapy, and adds to a growing literature demonstrating that variation in targets are likely to influence drug response, even in the absence of association to baseline characteristics of interest.

The EASE cohort is an interesting population for evaluating clinically relevant pharmacogenetic response to ezetimibe. The majority of patients on ezetimibe are on dual therapy with a statin, either taking the simvastatin-ezetimibe combination tablet or individually taking ezetimibe with one of the marketed statins. Many of the clinical trials that studied treatment with ezetimibe and a statin have been co-administration trials in which patients enter into a statin wash-out period and are then randomized to receive placebo or dual therapy. While assessment of pharmacogenetic response in this setting can be done, the results are confounded by the potential for NPC1L1 variants to affect statin response as well as that of ezetimibe.

The results presented here demonstrate that NPC1L1 promoter variation strongly associates with ezetimibe response. A significant association was identified between g.−18C>A and response to ezetimibe added on to stable statin therapy. In this cohort, patients who carried at least one copy of the minor allele had, on average, a 15% greater reduction in LDL-C compared to those with the homozygous major allele genotype. Homozygosity of the minor allele had no statistically significant additive effect on response (possibly undetected because the number of minor allele homozygotes was small) suggesting a dominant response model. Restricting analyses to patients representing the high and low (>40% reduction in LDL-C v. <5% reduction in LDL-C) range of the ezetimibe response distribution (n=120 and n=119 respectively) magnified the significance of the association. Significant association of g.−18C>A was also observed for other clinical endpoints analyzed among the complete set of genotyped EASE subjects, including total cholesterol, non-HDL-C and apoB, but not HDL-C or apoA1. These results are consistent with EASE data demonstrating that patients in the ezetimibe+statin treatment arm demonstrated significant reductions relative to placebo in total cholesterol, LDL-C, non-HDL-C and apoB, but not HDL-C or apoA1 (note that there was a significant increase relative to placebo in HDL-C in the EASE study).

Overall, SNP g.−18C>A accounted for approximately 1% of the variability in response among EASE patients who received ezetimibe. Given the complexity of cholesterol metabolism, the multiple homeostatic pathways controlling LDL-C, and the multiple environmental contributions to LDL-C levels (such as dietary fat intake, which significantly affects plasma cholesterol) the magnitude of this pharmacogenetic interaction is striking. There are few examples of pharmacogenetic interactions for variants with frequencies as high as g.−18C>A (˜15% in the general population) that are as pronounced. The HMG CoA intronic SNP that predicts lesser response to pravastatin is one of the most robust reported pharmacogenetic determinants for a statin ever reported, but identifies only a small percentage of statin users (˜5%).

Studies have demonstrated considerable variability in cholesterol absorption (Sudhop and von Bergmann (2002) Drugs, 62:2333-47. The association of a SNP in NPC1L1 with change in LDL-C suggests that variability in baseline LDL-C could be explained by DNA sequence variability in NPC1L1. No variants in this study associated with baseline LDL-C; however, all patients were hyperlipidemic and on statin therapy, confounding any link to baseline levels. There was, however, an unexpected over-representation of an NPC1L1 3′ UTR SNP in the hyperlipidemic EASE population as compared to the population control resequencing group. A striking three-fold increase in the frequency of g.28650A>G was found in the EASE versus control cohorts. This difference was confirmed by a re-genotyping of the re-sequencing cohort, with the same assay as was used in the EASE cohort. The average baseline cholesterol for patients enrolled in EASE was approximately 130 mg/dl, which for many of the subjects was assessed on a high statin dose; clearly an at-risk hyperlipidemic population. Lipid data are not available from the resequencing cohort, but these subjects were self-reported as healthy and were in general, age and sex matched to those in the EASE cohort. While other differences between the two populations could potentially explain the large increase in allele frequency in the hyperlipidemic EASE patients, one plausible explanation is that the g.28650A>G SNP predicts risk for elevated LDL-C. No association was found between baseline levels and the g.28650A>G SNP, but this analysis is confounded by statin treatment (i.e., LDL-C levels prior to statin treatment were not determined).

A 15% relative increase in LDL-C reductions translates to an additional ˜5 mg/dl decrease in absolute LDL-C levels. Epidemiological studies show that there is a 2-3% increased risk of heart disease for each 1 mg/di change in LDL cholesterol levels (Gould, et al., (1998) Circulation, 97:946-52. Based on such epidemiological data, the increased response seen in the g.−18C>A heterozygotes is anticipated to result in substantial reduction in coronary heart disease in a sizeable percentage of the population.

It must be noted that as used herein and in the appended claims, the singular forms “a”, “an”, and “the” include plural referents unless the context clearly dictates otherwise. Thus, for example, reference to “a complex” includes a plurality of such complexes and reference to “the formulation” includes reference to one or more formulations and equivalents thereof known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

What is claimed is:

1. A method of correlating a single nucleotide polymorphism or a haplotype in a NPC1L1 gene with the activity of a pharmaceutically active compound administered to a human subject comprising associating a single nucleotide polymorphism or haplotype in the NPC1L1 gene of the human subject with the status of the human subject to which a pharmaceutically active compound was administered by reference to the single nucleotide polymorphism or haplotype in the NPC1L1 gene.

2. The method of claim 1 wherein the status of the subject is determined by measuring a plasma component level selected from the group consisting of low density lipoprotein cholesterol (LDL-C), total cholesterol, non-high density lipoprotein cholesterol (non-HDL-C), and apolipoprotein B, before and after administration of the compound.

3. The method of claim 2, wherein the plasma component is LDL-C and the compound activity is the lowering of plasma LDL-C in the subject as compared to the level of plasma LDL-C in the subject prior to administration of the compound.

4. The method of claim 1, wherein the single nucleotide polymorphism is selected from the group consisting of g.−133A>G, g.−18C>A, g.1679C>G, and g.28650A>G.

5. The method of claim 1, wherein the single nucleotide polymorphism is g.−18C>A or g.1679C>G and the compound inhibits cholesterol absorption.

6. The method of claim 5 wherein the compound is ezetimibe.

7. The method of claim 1 wherein the haplotype is [A(−133), A(−18), G(1679)] or [G(−133), C(−18), C(1679)] and the compound is ezetimibe.

8. A method of estimating responsiveness of a subject to a drug affecting NPC1L1 function comprising:
obtaining a biological sample from a subject; and
determining the nucleotide base present at a position of SEQ ID NO: 1 in the biological sample wherein the position is selected from the group consisting of position 5,400 and position 7,096;
wherein the presence of an adenine base at position 5,400 or a guanine base at position 7,096 of SEQ ID NO: 1 indicates that the subject is more likely to have a higher than average response to the compound than an individual lacking the adenine base at position 5,400 or the guanine base at position 7,096 of SEQ ID NO: 1, and wherein the presence of a cytosine base homozygosity at position 5,400 or a cytosine base homozygosity at position 7,096 of SEQ ID NO: 1 indicates that the subject is more likely to have a lower than average responsive to the compound than individual lacking the cytosine base homozygosity at position 5,400 or the cytosine base homozygosity at position 7,096 of SEQ ID NO: 1.

9. The method according to claim 8, wherein the nucleotide base present at position 5,400 or position 7,096 of SEQ ID NO: 1 is determined by an assay selected from the group consisting of an allelic discrimination analysis, direct sequence analysis, differential nucleic acid analysis, restriction fragment length polymorphism analysis, DNA microarray analysis and polymerase chain reaction analysis.

10. The method according to claim 8, wherein the nucleotide base present at position 5,400 or position 7,096 of SEQ ID NO: 1 is determined by polymerase chain reaction utilizing two different primers that are complementary to two different portions of SEQ ID NO: 1.

11. The method according to claim 8, wherein the biological sample comprises a nucleic acid sample.

12. The method according to claim 8, wherein the drug affecting NPC1L1 function is ezetimibe.

13. An isolated polynucleotide consisting of at least 12 contiguous nucleotides of SEQ ID NO: 1 or the complement thereof, wherein the polynucleotide comprises a single nucleotide polymorphism selected from the group consisting of g.−133A>G, g.−18C>A and g.28650A>G.

14. A method of reducing cholesterol in a patient comprising the step of administering to the patient an effective amount of an NPC1L1 antagonist, wherein the patient is identified as having at least one SNP selected from the group consisting of g.−18C>A and g.28650A>G.

15. The method of claim 14 wherein the patient is identified as having a [A(−133), A(−18), G(1679)] haplotype.

16. A method for detecting a predisposition to a health risk level of plasma cholesterol in a human subject, the method comprising detecting in the human subject the presence of a polymorphism in the genomic sequence of a human NPC1L1 allele, wherein said human NPC1L1 allele consists of a guanine at position 34,067 of SEQ ID NO: 1, and wherein the presence of the guanine is indicative of a predisposition to health risk level of plasma cholesterol in the subject.

17. The method of claim 16, wherein the health risk level of plasma cholesterol is greater than the National Cholesterol Education Program Adult Treatment Panel III target level for the subject.

18. A diagnostic kit comprising at least one allele-specific nucleic acid primer capable of detecting a polymorphism in the NPC1L1 gene at one or more of the positions 5,285, 5,400, 7,096, and 34,067 of SEQ ID NO: 1 and an oligonucleotide probe for detecting a polymorphism in the NPC1L1 gene capable of hybridizing specifically to a nucleic acid wherein the nucleotide polymorphism in the NPC1L1 gene is selected from at least one of an A or a G at position 5,285 of SEQ ID NO: 1, a C or an A at position 5,400 of SEQ ID NO: 1, a C or a G at position 7,096 of SEQ ID NO: 1, and an A or a G at position 34,067 of SEQ ID NO: 1, and combinations thereof as well as their reverse complement.
